SEQUENCE LISTING (63 sequences)

SEQ ID NO: 1 - DNA, 22 bp, Artificial Sequence
  Description of Artificial SequencePCR primer
ggcagctctaccccgggatgaa

SEQ ID NO: 2 - DNA, 21 bp, Artificial Sequence
  Description of Artificial Sequence PCR primer
acagacctgagtgggacagga

SEQ ID NO: 3 - DNA, 21 bp, Artificial Sequence
  Description of Artificial Sequence PCR primer
tcctccttggcaggagtggta

SEQ ID NO: 4 - DNA, 21 bp, Artificial Sequence
  Description of Artificial SequencePCR primer
cagttacgggtgctcttgcgt

SEQ ID NO: 5 - DNA, 21 bp, Artificial Sequence
  Description of Artificial SequencePCR primer
atggcctccgaagcttctgtg

SEQ ID NO: 6 - DNA, 21 bp, Artificial Sequence
  Description of Artificial SequencePCR primer
tggtcgtgcggttcaatccct

SEQ ID NO: 7 - DNA, 24 bp, Artificial Sequence
  Description of Artificial SequenceTC5
aatctggccatggtctgctatttc

SEQ ID NO: 8 - DNA, 21 bp, Artificial Sequence
  Description of Artificial SequenceRT-PCR primer for TNG1
tgcatccctccagccaaggat

SEQ ID NO: 9 - DNA, 21 bp, Artificial Sequence
  Description of Artificial SequenceRT-PCR primer for TNG1
tggcctgcagaggctctcaag

SEQ ID NO: 10 - DNA, 22 bp, Artificial Sequence
  Description of Artificial SequenceRT-PCR primer for TNG2
gtgcctgtctcattcgcctctg

SEQ ID NO: 11 - DNA, 23 bp, Artificial Sequence
  Description of Artificial SequenceRT-PCR primer for TNG2
agtgggcacatgttacagcattc

SEQ ID NO: 12 - DNA, 21 bp, Artificial Sequence
  Description of Artificial SequenceRT-PCR primer
gcatccaggactgtgccagca

SEQ ID NO: 13 - DNA, 22 bp, Artificial Sequence
  Description of Artificial SequenceRT-PCR primer
ttctgttagccttgctgtccgt

SEQ ID NO: 14 - DNA, 22 bp, Artificial Sequence
  Description of Artificial Sequence3′RACE primer for TNG1
ttgaacccaggtctcgtctgac

SEQ ID NO: 15 - DNA, 22 bp, Artificial Sequence
  Description of Artificial Sequence5′ RACE primer for TNG1
aacgtaggatgtgcacagagca

SEQ ID NO: 16 - DNA, 22 bp, Artificial Sequence
  Description of Artificial Sequencemurine Tcl1b primer
gagaacggtcaggacccaaacc

SEQ ID NO: 17 - DNA, 22 bp, Artificial Sequence
  Description of Artificial Sequencemurine Tcl1b primer
caggctatcaagacctttactc

SEQ ID NO: 18 - DNA, 23 bp, Artificial Sequence
  Description of Artificial Sequencemurine Tcl1b primer
tcaacctcgcatattactatgtc

SEQ ID NO: 19 - DNA, 23 bp, Artificial Sequence
  Description of Artificial Sequencemurine Tcl1b primer
caaaggcacaaagtgagcaagag

SEQ ID NO: 20 - DNA, 23 bp, Artificial Sequence
  Description of Artificial Sequencemurine Tcl1b primer
aatgttggaaacttctcactcat

SEQ ID NO: 21 - DNA, 23 bp, Artificial Sequence
  Description of Artificial Sequencemurine Tcl1b primer
actggaaacttgttctcattcac

SEQ ID NO: 22 - DNA, 23 bp, Artificial Sequence
  Description of Artificial Sequencemurine Tcl1b primer
cacttgcagcatatgaccacaat

SEQ ID NO: 23 - DNA, 21 bp, Artificial Sequence
  Description of Artificial Sequencemurine Tcl1b primer
cctggtctgcacaagagatga

SEQ ID NO: 24 - DNA, 22 bp, Artificial Sequence
  Description of Artificial Sequencemurine Tcl1b primer
ctgtccacttgtggaagttaat

SEQ ID NO: 25 - DNA, 23 bp, Artificial Sequence
  Description of Artificial Sequencemurine Tcl1b primer
cacttgtggcagatgaccagata

SEQ ID NO: 26 - DNA, 22 bp, Artificial Sequence
  Description of Artificial Sequencemurine Tcl1b primer
ccaggagcctactccccagcag

SEQ ID NO: 27 - DNA, 21 bp, Artificial Sequence
  Description of Artificial Sequencemurine Tcl1b primer
gtggcagatgaccacactctt

SEQ ID NO: 28 - DNA, 23 bp, Artificial Sequence
  Description of Artificial Sequencemuring Tcl1b3 primer
cattactatggctgattcagttc

SEQ ID NO: 29 - DNA, 21 bp, Artificial Sequence
  Description of Artificial Sequencemurine Tcl1b3 primer
ggaatgagactctcagggcac

SEQ ID NO: 30 - DNA, 22 bp, Artificial Sequence
  Description of Artificial Sequencemurien Tcl1 primer
cctgggcaaggcagacaggagc

SEQ ID NO: 31 - DNA, 22 bp, Artificial Sequence
  Description of Artificial Sequencemurine Tcl1 primer
tgcttcttgctcttatcggatg

SEQ ID NO: 32 - DNA, 21 bp, Artificial Sequence
  Description of Artificial Sequencemurine Tcl1 primer
ttcatcgttggactccgagtc

SEQ ID NO: 33 - DNA, 21 bp, Artificial Sequence
  Description of Artificial Sequencemurien Tcl1 primer
aattccaggtgatcttgcgcc

SEQ ID NO: 34 - DNA, 21 bp, Artificial Sequence
  Description of Artificial Sequenceactin RT-PCR primer
gtaccaccagacagcactgtg

SEQ ID NO: 35 - DNA, 22 bp, Artificial Sequence
  Description of Artificial Sequenceactin RT-PCR primer
gacccagatcatgtttgagacc

SEQ ID NO: 36 - DNA, 20 bp, Artificial Sequence
  Description of Artificial Sequencemurine RACE primer
aagccatctataaggtcagg

SEQ ID NO: 37 - DNA, 24 bp, Artificial Sequence
  Description of Artificial SequencePFGE primer for mouse
tgctaggaccagctgctccataga

SEQ ID NO: 38 - DNA, 1152 bp, Homo sapiens
gaggcgggtcccggttgcagacttgccatggcctccgaagcttctgtgcgtctaggggtgccccctggccgtctgtggatccagaggcctggcatctacgaagatgaggaggggagaacctgggtgactgtggtcgtgcggttcaatccctcgcgtagggaatgggccagggcctcccagggcagcagatatgaacccagcatcacagtgcacttgtggcagatggcagtgcatacccgggagctactctcctccggccagatgcccttctcccagctgcccgccgtgtggcagctctaccccgggaggaagtaccgagcagcggattccagtttctgggaaatagcagaccatggccagattgactctatggagcagctggtcctaacatatcagccggagaggaaagactgacactgggagtggctggccctgctggccctgcctcttctggcctggtgtctcctcatgccccctcagtgaggatcttcatgtacctgctcttctgtttgcacacccagcatagcctccttgcaggcagaaggcagtagggcccctgcacactcagtttctctcgttttccttagttatcagtcctgtcctgtcccactcaggtctgtacttagggcagctggcctggatgggcttcactggggccctgtctgtgtgctgagccagtttcccctgctggctgcaagctgtgggttctttctcctctgtgcccctcatgctgatcttctagatgccactcccaaatccccttcatacccaccaggatgtgtgcccagccaggcctccagcacccccagtgcagctcgtgattggaaactcaccatcggcaggcagtggttcggtttaagagatggcattagagggagcccagtctggatgtggacttggatgccctgtgggtatcagttctgctgacactttggcccgaaatagatccagtgctgagcaagcaatgtacaccggagcctcagtgagcccatctgcacagtggggagcatggagggatgggtttggcctgtgcttctgcttattcagtccttcagctcacggaagggatgctagtccgtgaaggtgacctcacagtactggttaattaaactttattgctcactgtcaaaaaaaaaaaaaaaaaaaaaaaaa

SEQ ID NO: 39 - protein, 128 aa, Homo sapiens
MASEASVRLGVPPGRLWIQRPGIYEDEEGRTWVTVVVRFNPSRREWARASQGSRYEPSITVHLWQMAVHTRELLSSGQMPFSQLPAVWQLYPGRKYRAADSSFWEIADHGQIDSMEQLVLTYQPERKD

SEQ ID NO: 40 - DNA, 6486 bp, Homo sapiens
tcctcctcctccccctcctcccccgactggcaccgcccccactgccggccccgcccccactgccggcccgggccccacccacgccggagctgctccatttaaggagattgcgcagctggaaagctacacgtgtgagcctagaggcgggtcccggttgcagacttgccatggcctccgaagcttctgtgcgtctaggggtgccccctggccgtctgtggatccagaggcctggcatctacgaagatgaggaggggagaacctgggtgactgtggtcgtgcggttcaatccctcgcgtagggaatgggccagggcctcccagggcagcagagtgagtcctgggcacgaggggaggctgtggggagggctgcgcactgacccctgcccgtgtgggaccgcggtgggggtcagagggggccgttctcacccgcactggaaaactcacttctgtgcaggtctaggagcgcagcaatgtccatgcccagccctggccccaggaacaccccccgtaaagggaccacaggcacaagcttatccacatgagataatgtggtcctgcgtggtgaagccgaggctaaggtagctcagggcttagtgccattcccagtgcctgctgggaaggcccacaaatggggcagctattgagctgggctttgtgggatgagtaggagttctccaggtctagaaaggaggcaggagtagtataagcaaaagcattgcagcctggaggcaccaggtgggccaacaggatgaacatgacattggtgtcagattactgatctgcaaaatgagaataatatacctctgtggcaagctagtcacagacatgctcacatacatggctcaccgcctgaatggcctggggaagcatttgactgataacagattctggaaattaattcaggaggcttgggtggagtcctagattctttacttttcaaaagctccccaggtgataatgataatgactcaggaaacggctgtagatgagggctttagatcacagccagtctttgagggatgaagtaaatacagtagcgtctctggtgtgggtggcggtggggaattgattccaggaccgactgtggatgctcaagtccctgatagaaaatgacctgggtagtaattacatataacctcagcgcatcctctactatatttgaaatcagattactaataacacctaatgctacacctacacatcacttcaagctctgcttttgggaactttgtggaatttctttttttccccaaatatttttaatctgaggttagtcgaattcatgggtgcagtatccatggaaatggggggctggctgtaccttagtgtaatgtggtaaaagcatatccggatatttaaaatgccatttagggctgggcgcggtggctcacgcctgtaatcccagcactttgggaggccgagatgggcttatcacgagatcaggagatcgagaccatcctagccaacatggggaaaccccgtctctactaaaaatacaaaaaattagccgggcgtggtggcggacgcctgtagtcccagctactcgggaggctgaggcaggagaatggcgtgaacccgggaggcgaagcttgtagtgagccgagatcgcaccactgcactccagcctgggtgacagagtgagactccgtcccaaaaaaaaaaaaaaaaaaaaatgccgtttaggtcttcgtaaacaattcactgcctgtttgtttgttttttgagaaagtcttgctctgttgcggctggagtgcactggtgtgatgttggctcactgcaacctccacctcccaggctcaagtgattctcatgcctcagcctcccgagtagcttggattacaggcgatttttttttacagttaattttttttgttattttcaggagagacaaaagtttaatcatgtgggccaggctggttttgaactcctgacctcaagtgatctgcccaccttggcctcccaaagtgctgggattacaggtgagccacctcgcccagccagttcactgacactttaaacaatataacacatttcctaaaaaaagttcaaataggttatttcaaaaaatgttggtagagaacatggaaaggcttttctgtacatacactaaataaagcatgcaaaaattgtggagcaaatattttaagtttttcaaaagcctgaaaaagtgttaatggagggcactgtaaaatggtgcagccactatggaaaacaggatgaggatttctcaaaaaaagaattacggcataatccagcaatgccacttctggatatatacccacaagactctgaagccggaacttaagcatgtattcatacatccatgttcacagcagtatcattcatactagccaaaaggtggtggcagcccccgtgtccattgatagatgaatgggtaaacaacacaaaccatgaagtattcacccttaaaagtcagacacacggatgaaacttggagccattatactaaatgaaatatgccagtcacggaaggacagattctcttgtatgaggtactcagagtggtctcattcataaagtggaatggtagctgccaggggctggagggagtcgaggatgggaagttaatgttagtaacaggtacggagtctcagtttgggaagataaaaagttctggaggtggatagtgccgacggttccacatgtcaatgcacttaatgccaccaaactgtactcttaaaaacagttgaccgggcacggtggctcacgcctgaatcccagcactttgggggaccgaggcgggcggatcacaaggtcaggagatcgagaccatcctggctaacacggtgaaaccccgtctctactaaaaatacaaaagaattagccgggtgcggtggcgggcgtctgtagtcccagctactcggggggctgaggcaggagaatggcttgaacctgggaggcggagcttgcagtgagctgagatccagccactgcactccagcctgggcgacagagcaagactccgtctcaaacaaaacaaagcaaaacaaaaaaaacagttaagattttttttttttttaaatgattcagtggaaatagaatggattcttcaaataacttagccacgggtgggataagggacctacttagtaagtattttttccccttctttcttaaaaatagatcgatgtcttagggtgggaattaggcttcctgggcgacacatctaatgcaaagatcagccacctttttctgtaaaggatctgatggtaaacattttccacttgagagctatgctcttgcagctactcagctctgctattgcagtgcaaaagcagctaaaggcaacggtaaaggaatgacggaaggagccttagtttatttacaataaagctttatttgcaaaagcagatgcaagccagacttagtttgctgatctctgatctacagtcagaatacacagagaaggagagattttgccgtataatttaaaatacttctctttgcaaaagcagtccataaaaaaagtgaggacaacaaactgagaaaaattattcacaacatgtctgattgatagagcactaatattcttaattcaaaaagacattttatcacaaaagaagacaaatacttagaaaattgtgcaaaagactttccattttgttgcataacgtaggaagctttggttttacttttcctatcatctttctaacttccagtaccagcctaattttgttatttttattattatgtatttattttgagacagagtcttgctctgtctcccaggctggagtgcagtgacctgacgatagcttacaacagcctctacctcccaggttcaagaaatcttctcaccttagcttcccgagtagctgggactgtaggcacatgccaccatggccagctaattttttattttttgtagagacagagtctcattatgttgccgaggctggtcttgaactcgtggcttcnagcagtcctcctgccttggcctcccaaagtgttgggattacaggcataagccactgctcccagccttatttcgtatatttactataagtgtgtgaaggtcatgatcagaactgccatatattttggcgggaaaatctatcaccctcagatccaggagtccatggatatcttgtttttaaaacgaagatttaaaaaattacggcaatggcagagatggagccccaagagaatactcagctttaacccaaggtgttgacaggttggaaacagtggctaaatttggggattgcagtggggcgaggcagggtgcaggtcagagggggccagaagggccccagccatcctagatggagccacaagtaccagtgccaaggctcttggtctggaattctgaaaacatttacctctgaccctggcagcccactggccattgcttgtgtgcagcccagttggcagggaaccctatccatgatttgccgcctcttttctggtcccttcagtatgaacccagcatcacagtgcacttgtggcagatggcagtgcatacccgggagctactctcctccggccagatgcccttctcccagctgcccgccgtgtggcagctctaccccgggaggaagtaccgagcagcggattccagtttctgggaaatagcagaccatggccaggcaagtgtgtggtggttctaggtgaaagcgacaggtggcccctggtgactgccgtggccctctctcttctgtgcccctggcccccttggggttcttgtctgtcctcttcctgttgctcaagtcttccttcaaggaggcctgagtgtgtgtgggtggatcggtgcatgagttcccatgtgggatgcaggcagagtgggtgagggagggagggttgccttccctgggctagggaaatccataagctggagttcccacctgcctcacccctgcctgctgctgctgccagcctgcatgggcggccgttaaggccaactggaagagcatctcccagaggttctgatggctgctccctctcctgcagattgactctatggagcagctggtcctaacatatcagccggagaggaaagactgacactgggagtggctggtatgttggggccctgtgcgtctcggtgtagggatcagacgaaagtgagaagacctctcctcttttcagaaagacggcgtggcctcctcctccctgctgtttgctgagatttttcttacatagccacctgtcacctctgttccccagccccttggatgtgatggtacacagtgggtgggcccccataataagttcctaaagcatgggatctcatcgaataagactcatcatttaatccttgtgagaattttgtgaggtgtacgtgttaatgtcccatttcacgacgaaaagacaagactctggggatgggaatgacttcctcgagaccatacagccaggaaatagcggtgaatctagtgatctcgggtccctagatttaaccatggcactgaggtgccgtgtgacggtggccttggaggacccagcactgacccatagagggctcctctcagatgggcagcagcttggagcaggccaggcagggcctggtccattggaggggctggcactggacttgcctttgaccccagcagcttggatggggtgccgggctcccccatagttcactgactgtctcctttggtcttctcgcaggccctgctggccctgcctcttctggcctggtgtctcctcatgccccctcagtgaggatcttcatgtacctgctcttctgtttgcacacccagcatagcctccttgcaggcagaaggcagtagggcccctgcacactcagtttctctcgttttccttagttatcagtcctgtcctgtcccactcaggtctgtacttagggcagctggcctggatgggcttcactggggccctgtctgtgtgctgagccagtttcccctgctggctgcaagctgtgggttctttctcctctgtgcccctcatgctgatcttctagatgccactcccaaatccccttcatacccaccaggatgtgtgcccagccaggcctccagcacccccagtgcagctcgtgattggaaactcaccatcggcaggcagtggttcggtttaagagatggcattagagggagcccagtctggatgtggacttggatgccctgtgggtatcagttctgctgacactttggcccgaaatagatccagtgctgagcaagcaatgtacaccggagcctcagtgagcccatctgcacagtggggagcatggagggatgggtttggcctgtgcttctgcttattcagtccttcagctcacggaagggatgctagtccgtgaaggtgacctcacagtactggttaattaaactttattgctcactgtccacttttgtgctgaattggagcctctctttgacctctttctagcatagaaatggcagcttctggtaccgaaatgttaaggtaacattttaatgatccatttcatatttttccacactgggaaggaaattgtgattggtccattcagcagcaggac

SEQ ID NO: 41 - DNA, 1455 bp, Homo sapiens
gcttgctctggtgctagggtcagctagacaggtcggcaggagaccagctgctctagaagagcctggacaggatgaattggttctccttccacgtgcatcttgcatccctccagccaaggatttgaacccaggtctcgtctgactccaaatgcaccaagaggatggagcccagagtgacacagaggaagaggcccctggatggatgcatggggaagatcacgggaattacaagtgacatcctgaagtatgatcacaaatgtttcaaattaagccttcctgctaagtttccagaggtgtgtggctcggacgaagtgtttccagaccctgatctactgcatgtcctgcctgttgctggttctttgcagcagtccattgaccaatgctgtctacagcttgagagcctctgcaggccagggctgctctgtgcacatcctacgttactattcaaactgcacagcagcatgaaaaacaggcccttcttttctctcatttacacatatgtgaagaagacacagcaagtgagaaaacgtgaccgaaagccacgcggacaggtggccgcagggccaaacccaacttccgtgatgtgatctggctacagtttggagcccacatatttcaatagttatccccaggcctggcacgtcaaagaggctccagaaatgtttgctgagtgagtgatgaatcagcaatttctgattttctcctaaacttagagaataagtggcttgtctgcagtcacccagggcgtccctgacagaggtacaagtaaagcccagatctctgcatctctggttcaaggccttttgtactacacaccgctgtctctttgtcatcattaaaagcaaggctcaagggacatgttttctagccttagttccccatctacaaaatgggcctcatggaatggaatgtctccacttcactccagcatcaacaagtggggaattctgatggattcaattcgacttctttccatgggcgtgttctaagcagcctctttgttccagaagctgccctcagccagagttggataagccaatcctcactccccagcctcctctggatagggatgaagaccccactggggttggaagtgcagaggcagacaggtgtatggagtcacctgtaaattgattcaagtgagccaggaaagcagcaaaggaaagagaaacctgagtgacgacgtggtggaggaacagggctggaaagaggctgctggctgtctggcttcgcagctctggcctcctaatcagcctcgctcttgtctctggtgttctctggctcttgtccatctgtctgtgtttctttttgccagctattgactaatctttgctgaagctgagctagaattctggtgtttataagcaggtaactagctgagcactagttgataactttggagttagtggataaccacagggctgggctactaagctttttcagttgaaaaataaa

SEQ ID NO: 42 - protein, 140 aa, Homo sapiens
MEPRVTQRKRPLDGCMGKITGITSDILKYDHKCFKLSLPAKFPEVCGSDEVFPDPDLLHVLPVAGSLQQSIDQCCLQLESLCRPGLLCAHPTLLFKLHSSMKNRPFFSLIYTYVKKTQQVRKRDRKPRGQVAAGPNPTSV

SEQ ID NO: 43 - DNA, 2078 bp, Homo sapiens
ctcccaaagtgctgggattacaggcgtgagccaccatgcccggtcacctttgtcctgttttctacaggttagaagcaaggcacaagtcatacctacattcaaggagaggggaccatgcaaaggataaactgcagagagacaacagatttgcatcccagacacattcacactgggccaagagcagctgccattgcagattcgagtcacgtccttttttccttccttctccttcttggagttacaacagaagctggggaggtgagagtgcagaaaggacgtggatgaagcagagaggcacgtgcctgtctcattcgcctctggatctgctgcatccaggactgtgccagcacaaagtgggagcccgataaatatttattgaagaaatgaaaggtgacaaaaggcagaaggagggagaccccaaaaagagaccccaggctccagaagcagatgaggagacaaacagaagagcagaagaatggcagaatggcacagcagagagaagaaaaggaacatctgaatgttgagaggaatttggctgggggcggttggagaggagattagccactggacagccaaactccaggggaagatcatcttcccactccatcccccttccagctccccatccatcctgctgagagccacctccaccactcagtgaaaccactgcattcatccttcaagtttatgtgtgacctgattcttcctggacattggacaaagacctaggtccagtgaactgtctaacacttaagtcatccacggacagcaaggctaacagaatgctgtaacatgtgcccacttggactctgggagttgcagacacccacccgtaggtgctgccatgggtccggagcccagaaagtgcttgccctggctcctgcacctgcccgtctgcatgctccccctccggtaaggggtttgaggatgtggcggctgaacataagagctatgcccctgtcacacatcatgggacaggggtcagggaactctcccatttcactttcaaatattccaggtcccctttttgttcgggagccagagtctagtggagacagagatagaaaccctgaaataggatccaggcaatataaacctacacgctgtaagccaagtcagagtgaggctgatgtgataacaaaagtctgaatacaaggtgggagacaattattctgactgaaagggaggcaagagttctgccataggcttgatgctgtgtgtccctggcttcctaactcatttgcaaatggaaaatactattccattgtaggaaccttctttttccatttgtctggggaaggagaaagaatggctgggctgaatgaatttatcccttggtcctcttccaacaaagctcagctatgaaagataaatccagctctccccaccccctctcagtggagctggggagaaatcaaaagcccctctgccaataatgagaccaaagtttgcaagggcaggacgagcccgtgctaacagagaaagtgttgtttcctcaatttggttttagactgtcttgtcctatgggggagaaaagatctgcccttgggagaggtgccaactttatagatctattaataaaagaactggcaggcttacagttcttgccaatgaggaaacttgaatgagagaagccaggctcaaccttggccaacagactggagcccatcaccctaacttcaccccgcttctccttacccaaccatcaaaggctaggcagcacccacccagcagcttccacctggctgaagcctgcacctgcttcagaccaagggttagatggaaatttggcatgggaagagagggctcacctgtgggcaggatagactctatccaagaaggagaactgaaaaatgaaaacctatgagacaaggggtgatcctgaaggcaggcaggagaaagggctggagggagaggcactggggaatttttcctggtgaatactgaagttactagatgttttgtcttgcaaaactcaagggaaaactctcaaactctaatgtttgtctattctgtgtccaaactgtccttttgaaacggacccacctttcagtaaagaaacttgcattggcctgccc

SEQ ID NO: 44 - protein, 110 aa, Homo sapiens
MPGHLCPVFYRLEARHKSYLHSRRGDHAKDKLQRDNRFASQTHSHWAKSSCHCRFESRPFFLPSPSWSYNRSWGGESAERTWMKQRGTCLSHSPLDLLHPGLCQHKVGAR

SEQ ID NO: 45 - DNA, 4509 bp, Homo sapiens
gagcttgctctggtgctagggtcagctagacaggtcggcaggagaccagctgctctagaagagcctggacaggatgaattggttctccttccacgtgcatcttgcatccctccagccaaggatttgaacccaggtctcgtctgactccaaatgcaccaagaggatggagcccagagtgacacagaggaagaggcccctggatggatgcatggggaaggtgaggcctggaggtgatgtgcttgagattaccaggctcttctgaacctgggggaccttctcctatgatagccctgctcctccagacccagcaggtgatttccccaaaagccgcttacagtgaacgcacctgatagcaataacttaagcatcccctgagaatgacccttatggcaggtgcaggtgaatgtggtttggagttctgagctaaggaatctgggagtggccaacctggagattcattcctattccttatctatgaggaacatctgagctcctgtcccgtcctgtgcaacacggacagtacaggggatcaaggccctttgttttgggttaggtaaatgttgccaggtggacactgttgggggagggtgctaagtgaagatgctatatatgctgcaagctttttgtaagttgtgtggctctcctgcccagcccaccactgctagatgctctcccctgcatgtaagccccagtaaaactccatgtctccttcagcagccctgggtcctttcttcagcctctcaaacctgctgccatccccattggagtagataggggttcagcataacagcagccaccagctggggaaaggcgtggcatttggagctccctacatggtcttgactctaatatggagggaaagaaaacagattctgccctgctcagtaaagccccctactggcctcccaccctggactgtggacaaagcttttttttttttttgagacggagtctcactgtgtcccccaggctggagtgcagtggcgtgatctcagctcactgcaagctccgcctccccggttcacgccattctcctgcctcaggctcccgagtagctggggctacaggcgcctgccactacacccggctaatttttttttttttttttcatttttagtggagacggggtttcatcgtgttagccaggatggtctcgatctcctacctcgtgatctgcccacctcggcctcccaaagtgctgggattacaggcgtgagccactgcgcccggccaacaaagcatatttttttcctctgaggtctttaccctataactgcaagtggtttcatgacactgcacatgggttcttatctggggttggtagattcaactcaattccacaggtttactgggtgttgcctcccatgcttggagccaggaggatgcagggaaatggggctgccaccatacccgctcttcttatgatcccatcacatcattgcaccctttggggcctggcaccatgctctggcactttgcctccatcatctcctttaacctctgagctctgtcaggaaggcattactactatccctggaatacagacaggaaaactgagttggattgaggctcagagaggttaagtcacttacccaaggtcacaaagctagagagtggcaggccagcatccaaggccaggaacacgagctccctctgatccaaatggtgactctatgtttgatcagtgctctgagaactctccaaagatctttatcaactgtcatctcttcattgcccaggcccagtcagggagccacttggctgcggtcccgctgagatttaggggcaggacccctgatgtgaggtggaatgcttggtcacgctgttcaagcctccaccagtcactcctgactggttatggacactcaatgtgccccgtcctgtgtgcaatgcagtgtgggaaggggatcaaggaccttggctaatcccaggcatttccccagaatgtcccaggtgtggaccccttgggaggtgctgggaagcaggctggtgtcccacaggccttggttccatcccagctgtgctgtgctaagctgaaactctttgggcagcttgtttcacctcatgagacttctttttctgaactgttaaatgggatgattcgacctctgcttggcttttggtgacgattaaacgagatattagctgtaaagcatttcatcccgggtctggtatagaagaagcacttgataaatgaaagctctgttgagtatgatgatccgtcaccccaggcatcagtgggatctcacagcaggaacaatgcctaactcctaactgatgcccagtaaacatcgggtggatgcaatgtccaacaagacagatattcatgttcagttttacagaaaaggagacagaggccagaaaaggaagaaagtgcccaggtcagccggctagtaagaggcagagttgaaatttgaaccgggttctggctggctgcagctccactgctccactactgcactgtctgagagccaaaaggcaccttgtagtcaggcaggcagggagctggcttcttgctgtcctgctggatcctgccctagtgtggccctccctggaaccaggggtggggctggagggcagggcactgagagagatctcctgggaactgtaccatctgcacaaagggcacctgctgggcactctgctgaaagctgtctgcaaacaccacttcctgcctgcctccctgggggctctaagctcaggagggggtgagggcctatgtctgcacgtggtgaggcccctgctgggtattgtaccacaccttcattgtgctcagggtggccagtggaggcccttcctagactcgagctctggtcacaagcccaaccccctccttgctcctgtctgggctcacctggaatctcctgcctcactctgtggccagaggcacctttggaagcccaggtcacccctactactcatgcccctcatttggaaattttcataatctacctcttgttaatgattccatcccatctataatgcttttctatttttctgaatttttccccgataggctataactgctgcaaagatagagaattgcaatgattaatatgcatttatgtatttccaaaaagttaccagaatatcaaactcatgattttgtctatagtttggttagattcaagctatttttatatcataaaacaacttaaaatttttttatttttaatttttgtgggtgcagagtgatgtatttatggggtacccgagatgttagaatgaagctattaaatgtgagtgtgaacgttcacctttttgcagatcacgggaattacaagtgacatcctgaagtatgatcacaaatgtttcaaattaagccttcctgctaagtttccagaggtgtgtggctcggacgaagtgtttccagaccctgatctactgcatgtcctgcctgttgctggttctttgcagcagtccattgaccaatgctgtctacagcttgagagcctctgcaggccagggctgctctgtgcacatcctacgttactattcaaactgcacagcagcatgaaaaacaggcccttcttttctctcatttacacatatgtgaagaagacacagcaagtgagaaaacgtgaccgaaagccacgcggacaggtggccgcagggccaaacccaacttccgtgatgtgatctggctacagtttggagcccacatatttcaatagttatccccaggcctggcacgtcaaagaggctccagaaatgtttgctgagtgagtgatgaatcagcaatttctgattttctcctaaacttagagaataagtggcttgtctgcagtcacccagggcgtccctgacagaggtacaagtaaagcccagatctctgcatctctggttcaaggccttttgtactacacaccgctgtctctttgtcatcattaaaagcaaggctcaagggacatgttttctagccttagttccccatctacaaaatgggcctcatggaatggaatgtctccacttcactccagcatcaacaagtggggaattctgatggattcaattcgacttctttccatgggcgtgttctaagcagcctctttgttccagaagctgccctcagccagagttggataagccaatcctcactccccagcctcctctggatagggatgaagaccccactggggttggaagtgcagaggcagacaggtgtatggagtcacctgtaaattgattcaagtgagccaggaaagcagcaaaggaaagagaaacctgagtgacgacgtggtggaggaacagggctggaaagaggctgctggctgtctggcttcgcagctctggcctcctaatcagcctcgctcttgtctctggtgttctctggctcttgtccatctgtctgtgtttctttttgccagctattgactaatctttgctgaagctgagctagaattctggtgtttataagcaggtaactagctgagcactagttgataactttggagttagtggataaccacagggctgggctactaagctttttcagttgaaaaataaata

SEQ ID NO: 46 - DNA, 8621 bp, Homo sapiens
cctcccaaagtgctgggattacaggcgtgagccaccatgcccggtcacctttgtcctgttttctacaggttagaagcaaggcacaagtcatacctacattcaaggagaggggaccatgcaaaggataaactgcaggtgaagaacttgagagctattttagaagctgccttccacactttacaatctactttgcagctatactgagctcttagcaactccctgggcctgctgagcacctcttgcctttctgcctttgcgtatgctgttccctctgcctggaattccatgtcttctgcctttgcccacctcatcttaaggcctctgcttagatgcaatttctttgggctcttattagcagcatccaaatctgtgctggtttcttctcacgcttactctgcatcataatttttctgtttactcgttaatcccctcccactcctgccagtacctaaagtaaaatctacacgggcttggcacattgtggatgctcaatgaagatttgttaaatgagtgaatgattaaatgaaataagccccagcttggaacccagagctgtaatttccggttgtggtccctgtcccctgcccaggggctgtctaagggctcatctctcccaggagcccctgtatcatctcagtaactagaccagtaccatatccgacttcccctgactttgaaataattttcacctgcaatcaggtgcgcttccctagaacctctctcgctgttctttgcctttctccttcccccgccttgtatctccgtggtcctgccaaaaatcagaagctcattattaaaccagaagcttccttgcattatcccagagactacagacagaaaatatttgcttctatctcaactgaggactgacatagtaaagactccttgagatgttagctgtcaaggaggggaataaaagtttttcatctgcatgagtaattgtttcctgttttatccagagagacaacagatttacatcccagacacattcacactgggccaagagcagctgccattgcagattcgagtcacgtccttttttccttccttctccttcttggagttacaacagaagctggggaggtgagagtgcaggtgagagaggaggggggctctggaacatgtagcctgtgaggagggaaatcaggatggtgcttgggtggaggtgggagctgggccagggtcttggctggtcaggaaggctggtgaagcatgtactggggaggcagaggtcctgctttctttcctctctttccttcacacctacctcaacccactgtcgagtcctatcagctctgaaatagatccttcctccagcgccttctcacctctctctccaccattccagtccatgccgcctcatctcttactgggactttcacaacagcctcccgagtggtcttggcttctgctcgcaaatcctgcagtgccttctctgcggagccacaagctggatctttttaatccgctctcggtttctcacagcccttggaatgaaaccttggcctacagccctgtgtgacttgctacctgtccttacctcctaacctcatctaatatccccttcccctcctcgtaccggcctttctgtttctcagacactccaggcttgttctgctcttggaccttcacgctgtctttctgtctcctgggaatctgtgccccctctgccagatcatgcagctggctctctctgcccaaataccctcttccgaggggtcttccttggccctgtgtctgacataggtaccctcctccatcactccagcccattacagtgccataacacttttattgccatctagagtaacatcagttggttttgtttatcatctgccaccctgctagaaaggacgtggatgaagcagagaggcacgtgcctgtctcattcgcctctggatctgctgcatccaggactgtgccagcacaaagtgggagcccgataaatatttattgaagaaatgaaaggtgacaaaaggcagaaggagggagaccccaaaaagaggtaaatttggtatcttagcaagtgggtgactaccactgtctgggggaaagctgatacacgaggacttgacaataattacagcagtaacaatcgctactattttgttttgaaatgaattttagtttctgaaattgtccaaagcacttttatcaatagaaacttgttacacatccctccaacagggagaagcagcacaggaagctgccccctcaaacactaaaggctcctggagctattcacagtgctggggaacaaccagaggaggaggaggacagagggagagaatactttttttaaaaatcccaaacaaaaccaaattctatacatgtatatctcgagtgagtaaatgtgtaacactcagcagtctcctacacttgccaggttttgtggcttcatgcagtaatataaaatgtgcttcacaaagctaggagttggcacagaatatagggttctgattcatattttagttttacttttttgtagagatgggatcccactatattgcacaggctggtctcaaactcctgagctcaagcgatcctcctgccttggcctcccacggtgctgagactacaggtgtgagccaccactcccagcctgatttatattttataaagaggcaacgatggagtacacagctgagcaatctcttgcatttctggaccttgagcatcaggaagcttagggagggtgaaaactcagaagaaagctcagagttgagacctttgattttttgcccagagagtccagctaacacactttataacccaaaaggccttacaaatatttcagagcagaggcagcatttttgcactggataaacaaaggtacggtaatttcttcatagtttattttcattacaagtaattcttttcctgaagaaaaatttatatttcagatttatttcccctctcaaaggggaggggagttataatattctgtttccatagcaccattcctcaagatcaaacccaggcaggcatttttacagcacacacttccatatgtcattgctaatcctcgcaaccttgaggaaactgaggtgcaaaggggttgaggaatgtgttcaccatcccaaagccaacgaagtgccagaacccaaatccaagcttggctgtctcatccccaaggacgtactctttatgccgttccttatgacttttcatgcagcaggcgcaaggcagcaagaatttggctcttctgacatttatcaggcacctggtatgtgccaggcctactgtaaaccccaagatgacatggtccatgtctatccattcagcatcagagagactgttatcaggacttgaggcctgagacacagccctgaatcagatgcaggcctctggtgtttgcaaaggatgcgtgagtatgtgtatgtgtatgcgtgcattgtgtgtaggggtgtgtgtatgttgggggttgggggcagaaaatgctccctgaactgttggtattaaggcagatctttgatggagggactggggtgtggagacttgaaccctagtttcgaagtggggacctcgaacactagtatggccagcagacaggctggagttgctgaattgtcagctaggtctttaatccactccccttctctaaatcagggtggaattggactcagggctggttcagggctgaggtttccgggggatgaatgattccagacagtgggaaggggtgatggatgatgaacctggcaagtcacaggactatggacttggctgtatgttgcatgtatgtgtaagatctgagttgagacctttgatttttgcccagaaagtccagctaacatactatataagccagtgagaccagccagtgtctgttcattgcctctgccttgcccctatgcctgcccgcctcctatagcaggtccctatagcataatgaaaatgcgaacaaccttttctctggacactggggaatctagtctggtggataaagatggatggctgttaaacgatcacatcttgggctctgtgtgttcagtgaagcctcatttaccagatctctctgatgagaaaggtgaatggtggggacaaaatacatgctgttatcagaagtctggcgggggccctgtgcagtggcaagcacctgtagtcacagccactcgggaggcagaggtgggaagatcgcttgagcccaggaggttgaggctgcattgagctatgatggcaccattgtactccagcctggttgacagagtgagaccctgtctaaaaaaggaaaagaagtttagggggaaagcttttaggaggtagatgatggttaattttaattccacctaacattttaaactttggtatgtcaaaaggtgagaattgccagtgtcaaggcatggaaaccaaaggaaaaccagagggttgagaaggtgggaagggccgcagtgtggcaggcggcagtttctgggatggcttctactaaaggaactcatcatccaccaccttcaggaaggaggctcctggggccctttaagagagcagtttcagtggaggctgcaggcaggttggcagaaacaagattatgagatggtctgtaccaggtatcaggcttgagggttagtgcctctagacttctctgagaagtctagctttgaagggaaggctggagagctagcacaatactcaataataacaagacctaaggcacttactgtgtgccaggcactgttctaagaactcagcacacttcagctcatttaatccttacaactatacaaagtttgtagtcttttcgttataccttttgacaggtgagttacttgctggaattcacgtggctagtaaatactggagctgagatttgaaagcaggcaatctggctccagaatctgtgttttcaacccacatactatattgaccatcatgaaaagatggtggtgagcaggcttgtttctaaggatgggagggtcctcataatatttgtatttacagagaggagggagaggtgggttacttcctctcccaaccccagaaagccaggcatgttggctggcatccctgggacctcagatattcaagcaaccatggccttacaaatcaggcattctatttattagagttgtgcagtgaggtcccctccatttcatccccagaatatctcctagacaagttggatcctgcagggcccgagtataaataagatggtgccctctgttgttatggagtagcttggagctgggccccagatgccctgtgtggattagatgagtgattagtatttggcaatgtagcttgagaggcttgctgagttactggggcatatgtccccagctcacgagtaccagcctgacttctgtcaatcaatcagtgaacagggtttgcagcgtccaccaggtacctgtggtgtagtagaagagagtttgaactttggaatcaaacatgctgaagttcagattccagctcctccactaactacctctgtgatcttggctaagtgaccgtcactttttttttttttttttttgagacaagatctcattctgtctcctaggctggagtgcagtggcatgatcatggctcactgcagcctcaacctcccgaactcaagagatcatcccacttcagcctcctgagtagctgggaccatggggcacctgccatggcatcaagctaattttttatcttttgtaaagatgggatttcgccatgttgcctaggccagtctcaaactcctgggctcaagcgatccacccacctcagcctcccaaagtgctgggattacaggtgtgagccaccgtgcccatcctgatggttgctttcagagccttggttcttcatctctaaaacaaagataggaatactgcctagtttccatggggagcttgtaacacatgcgggcctctctcccctcaccccagtctttggttagacacttactatttgcaaagacccaatttcttcatcataagaaggctgccaccacacttgagggagtgacccatttacaaacaattgcttccttctgggtcattctgatggattaaatgaagaaccatttaaaaccacagcccaaagggccagagggagaggcagcagagggaagatgcaggtggatcttcatgcactgcatgaggaagctttgtaccatggtcaggaaaagctccaggcacacaaggcagcctccatggccaatcagagtgcacctctcacctcggcaactctgctaacctaaatagaccagtcacacagcagagctgcaaaagaagacatgataattacagagaccccaggggaccgtctgtgcggactgatgctttgtttctcctgacccctcagacctctctatttcactgcaaactctcaatacccagggcctttgttttgctgtgaacactttcattcctgagtacattttctatgaggagttggcggaagagagtcagaatgaattatattttcagcacacctctgggttcatgttgaacttggctagaactgttttgacccaggaaacaatttgactaaaggatatttcaatccaacaaatactctctgggcccttccaatgtttctggcactggtttgatgctgaggacatagagatgaataagaaatggtctctgtcccccagctgataaattatcattgatgatctaccgtgtgccagggactttacaaatgtgattaccatttcttacaacatagtaaggtagatagcatttttgtgttacaggtgaggagtaggaagttcaaaaagtttataaggtcaaagtcacacaactagtcagatgtagaactaacctttggatttgggactgttcaccatcaaatcctatatgcttttgatacagacaggagacccagaaatactgggtagaagagggtggttccctggcaaaggccctaccctcaaacctggaaacctgcagccctaaatgggaagaggcattcctgttttcatgcccaaaagttgccttttggcccgtcatgtcccctatcctatacctgtataaaccccagaccccaggctccagaagcagatgaggagacaaacagaagagcagaagaatggcagaatggcacagcagagagaagaaaaggaacatctgaatgttgagaggaatttggctgggggcggttggagaggagattagccactggacagccaaactccaggggaagatcatcttcccactccatcccccttccagctccccatccatcctgctgagagccacctccaccactcagtgaaaccactgcattcatccttcaagtttatgtgtgacctgattcttcctggacattggacaaagacctaggtccagtgaactgtctaacacttaagtcatccacggacagcaaggctaacagaatgctgtaacatgtgcccacttggactctgggagttgcagacacccacccgtaggtgctgccatgggtccggagcccagaaagtgcttgccctggctcctgcacctgcccgtctgcatgctccccctccggtaaggggtttgaggatgtggcggctgaacataagagctatgcccctgtcacacatcatgggacaggggtcagggaactctcccatttcactttcaaatattccaggtcccctttttgttcgggagccagagtctagtggagacagagatagaaaccctgaaataggatccaggcaatataaacctacacgctgtaagccaagtcagagtgaggctgatgtgataacaaaagtctgaatacaaggtgggagacaattattctgactgaaagggaggcaagagttctgccataggcttgatgctgtgtgtccctggcttcctaactcatttgcaaatggaaaatactattccattgtaggaaccttctttttccatttgtctggggaaggagaaagaatggctgggctgaatgaatttatcccttggtcctcttccaacaaagctcagctatgaaagataaatccagctctccccaccccctctcagtggagctggggagaaatcaaaagcccctctgccaataatgagaccaaagtttgcaagggcaggacgagcccgtgctaacagagaaagtgttgtttcctcaatttggttttagactgtcttgtcctatgggggagaaaagatctgcccttgggagaggtgccaactttatagatctattaataaaagaactggcaggcttacagttcttgccaatgaggaaacttgaatgagagaagccaggctcaaccttggccaacagactggagcccatcaccctaacttcaccccgcttctccttacccaaccgtcaaaggctaggcagcacccacccagcagcttccacctggctgaagcctgcacctgcttcagaccaagggttagatggaaatttggcatgggaagagagggctcacctgtgggcaggatagactctatccaagaaggagaactgaaaaatgaaaacctatgagacaaggggtgatcctgaaggcaggcaggagaaagggctggagggagaggcactggggaatttttcctggtgaatactgaagttactagatgttttgtcttgcaaaactcaagggaaaactctcaaactctaatgtttgtctattctgtgtccaaactgtccttttgaaacggacccacctttcagtaaagaaacttgcattggcctgcccagcc

SEQ ID NO: 47 - DNA, 1040 bp, Homo sapiens
gtgattcccctgtctgaccgcatttaagaaggcaggcaactggaaggcttcattactatggctgctgcagcttttgatcccctggggccacttccagtctacctggtctccgttagactgggcatctatgaggatgaacatcatagagtgtggatagttgcaaatgtggaaacttctcactcatcacatggcaacaggaggagaacccatgtaactgtccacttgtggaagttaatcccccagcaggttattcccttcaaccccttgaattacgactttctacccacgacgtggaaattagagtccaggaacatatactgggcaacagatgggactcactggagattactggatcattcccagttgggtgacacagagcaactgatcctgatgctggtgttagggtgactgactgatggaagctgctgggaaggtcttgatagcctggctggctcctgctcctgtgccctcgggtagactttgcacccacccctctgtttgttcctcaagtttctagttctacagcaacttctgtcatggtcagacctactttttacatgatctactttctgtgagcctggtttgggtcctgaccgttctccctagctggagacaaactgcatcttctcttgcccactttgtgtctctggccctgaccttatagatggcttttcttaagacccctcctccattcaaactccttccacctgggcttctagtgcttccagtggtaggagatttgccccccatttaccttaggatgtctacaggtgcctctggaggaggtacagatagggactctatgccctgtgggcctttgacctatcactggcaagaagtttgcctggtgctgggtagaggacactctgtgctcagtagcttaaaggctcagtgtgagacttacagagaacatctggagtacttgtccaggtgctcagttccttggctccaggaagtgaatctgaccctaggcctctcccaccccaacttgtttttaactaattaaactttactgtttttacttaaaaaaaaaaaaaaaa

SEQ ID NO: 48 - DNA, 1020 bp, mouse
gtcattcccctgtctggccgcatttaagaaggcaggcaactggaaggcctcattactatggctgctgcaggtttttatcctccgaggctactcccacaagttctgatctccacaggaccgggcttctatgaggatgaacatcatagactgtggatggttgcaaaactggaaacttgttctcattcaccatattgcaacaagattgaaacatgtgtaactgttcacttgtggcagatgaccagatacccccaggagcctgctccctacaatcccatgaattacaactttctacccatgacgtggagattagcatccatgaacacataccggggaacagatgccatgcactggagattactgaatcattcccaggttggtgacacagtgcaactgatcctgatgctggagtaaaggtcttgatagcctggctggctcctgcccctgtgcccttggggtagactttgcacccacccctctgtttgttcctcaagtttctagttctacagcaacttctgtcatggtcagacctactttttacatgatctactttctatgagcctggcctatctctgtcctgaccgttctccctagctggagacaaactgcatcttctcttgcccactttgtgcctctggccctgaccttatagatggcttttcttaagacccctcctccattcaaactcctccacctgggcttctagtgccttgtgtggcaggagatttgtccccatttaccttaggatgtctacaggtgcctctggaggaggtacagatagggactctatgccctgtgggcctctgacctatcactggcaagaagtttgcctggtgctgggtagaggacactcagtgctcagtagcttgaaggcacagtgggagacttacagagaacatctggagtacttgtccaggtgctcagttccttggctccaggaagtgaatctgaccctaggcccctcccaccccaacttgttttaaagtaattaaactttactgtttttacttaaaaaaaaaaaaa

SEQ ID NO: 49 - DNA, 1058 bp, mouse
gtcctgcccctctctgaacctcatttaagaaggcacgcaaccggacggcctcattactatggctgattcagttcattttccctggatgccattcccacctcgcttcctggtctgcacaagagatgacatctatgaggatgaaaatggtagacagtgggtagttgcaaaagtagaaacttctcgttcaccatatggcagcaggattgaaacatgtataactgtgcacttgcagcatatgaccacaatcccccaggagcctactccccagcagcccattaataacaactctctccccacgatgtggagattagagtccatgaacacatacacgggaacagatgggacatactggagattactggatcattcccagatgggcgacacattgcaactgatcctggacatagtaatatgcgaggttgactgaatgatggaagctgatgggaaggtcttgatagcctggctggctcctgcccctgtgcccttggggtagactttgcacccacccctctgtttgttcctcaagtttctagttctacagcacctcctgccatgattagacctgctttttagattatctactttctgtgagcctggcctaactctgtcctgaccgttctccctagctggagacaaactgcatcttctcttgctcattttgtgcctctggccctgaccttatagatggcttttcttaagacccctcctccattcaaactcctccacctgggcttctagtgccttgtgtggcaggagatttgtccccatttaccttaggatgtctacaggtgcctctggaggaggtacagatagggactctatgccctgtgggcctctgagccatcactggcaagaagttcgactggtgctgggtagaggccactcagtgctcagtagcttgaaggcacagtgtgagacttacagagaacatctggagtacttgtccaggtgctcagttccttggctccaggaagtgaatctgaccctaggcccctcccaaccccaacttgtttttaactaattaaactttattgtttttacttaaaaaaaaaaaaaaa

SEQ ID NO: 50 - DNA, 1040 bp, mouse
gtcattcccctgtctgaccgcatttaagaaggcaggcaaccagacagcttcattactatggctgattcagttcgttttccctgtatgccattcccaccttgcttcctggtctgcacaagagatgacatctatgaggatgaacatggtagacagtgggtagctgcaaaagtggaaacttcttctcattcaccatattgcagcaagattgaaacctgtgtaactgtccacttgtggcagatgaccacactcttccaggagcctagtcccgactctctcaagactttcaactttctacccaggacatggagattagagtccaggaacacataccggggagcagatgccatgcactggagattagtgaatcattcccagttttatggcacagaggaactggtcctgatgctggattcaaggtaactgactaatggaagctgctgggaaggtcttgatagcctggctggctcctgcccctgtgcccttggggcagactttgcacccacctctctgcttgttcctcaagtttctagttctacaacacctcctgccatggtctttttacatgatcttctttctgtgagcctggcctatctgtggcttgacggttctccctagctggaggcaaactgcatcttctcttgctcactttgtgcctttgacactgaccttatagatggctcttaagacccctcttctcttcaaaatcctacacctgggcttntagtgccttgtgtggcaggagatttgtccccatttaccttaggagttctagaggtgcccctaaaggaggtacagatagagactctatgccgtgtgggcctctgagccatcactggcaagaagttcgcctggtgctgggtagaggacactcagtgctcagtagcttgaaggcacagtgggagacttacagagaacatctggaggacttgtccaggtgctcagttccttggctccaggaagtgatgctgaccctagacccctcccaaccccaacttgtttttaactaattaaactttactgtttttacttaaaaaaaaaaa

SEQ ID NO: 51 - DNA, 1050 bp, mouse
gtcattcccctgtctgaccgcatttaagaaggcaggcaaccagacagcctcattactatggctgctgtgtctgttgatccccagaggccactcccagtcctcctggtctctgttagcctgggcatctatgaggatgaacatcatagagtgtggatagctgtaaacgtggaaacttctcattcatcacatggcaacaggattgaaacatgtgtaactgtgcacttgcagcatatgaccacactcccccaggagcctactccccagcagcccattaataacaactctctccccacgatgtggagattagagtccaggaacacatacacgggaacagatgggacatactggagattactggatcattcccagatgggcgacacagtgcaactgaccctggacataataataggcgaggatgactgaatgatgggagctgctgggaaggtcttgatagcctggctggctcctgcccctgtgccctcggggtagactttgcacccaaccctctgcttgttcctcaagtttctagttctaccacacctcctgccatgactagacctgctttttagattatctactttctgtgagccttgcctatctctgtcctgaccattctccctagctggagacaaactgcatcttctcttgctcactttgtgcctctgacactgaccttatagatggcttttcttaagacccctcctccattcaaactcctccacctgggcttctagtgccttgtgtggcaggagatttgtccccatttaccttaggatgtatacaggtgcctctggaggaggtacagatagggactctatgccctgtgggcctctgagccatcactggcaagaagttcgactggtgctgggtagaggacactacgtgctcagtagcttgaaggcacagtgggagacttacagagaatatctggagtacttgtccaggtgctcagttccttggctccaggaagtgaatctgaccctaggcccctcccaaccccaacttgtttttaactaattaaactttactgtttttacttaaaaaaaaaaa

SEQ ID NO: 52 - DNA, 6921 bp, mouse
gtcattcccctgtctgaccgcatttaagaaggcaggcaactggaaggcttcattactatggctgctgcagcttttgatcccctggggccacttccagtctacctggtctccgttagactgggcatctatgaggatgaacatcatagagtgtggatagttgcaaatgtggaaacttctcactcatcacatggcaacagggtgagtttcagggctccagacaggctattccccaactcttacccctgtgagctagggtgtgtttacgtgtttgtgtgtgtgtgtgttgtgtgtaggtatgtacttttattaccgtgtcagaaaatagctgatacaggtctctgaatagaaccttgtgtatacctaggcccataccctggagctcccttggagacctcacaggcccaactaatacacaggtgcagagccctagcattgagaactccgttacagatagtcagagagaacatgctgagagagctttgttaggctgggctttgtgggatgagtaggagttctacaggtcagaaaaaaaaaaaaaagcactctggtgccaggcagattccctggaaggagcaaatgtgagtgccagcactactttccgcatttctgcaaatcataattcatctgctgctgttgctcccagacttgagtggcctgaagaagtgtttgacagacaagggatccttacactaggagctgactacaggacttgggggaagtcctaatgggttctgtgatctgcaggtgatgtagagacccacactgctggaaacctgctctggaccatggctttagtccacagcaggaccttgggggctaaaacactctagtggaaagctctgggcattaaaactgctccccagggtcgtggaaaagactatgggccaccaccaccacccccactgccctgggggtgtggaggacttgggggcattaggtctctttagctgccagggttttcctgcacacccaccccctgtcctaacctccagcccagcagcccttccctgcagctcccctgactccctgaatacctcagccattttggcaatgggactctcttggtccccccctcccctctctccttgacttacaaggcccagatcaaggtcatgttcactctggactctcccaaatgtctgtttttgcctgtgttctcctttttaatttactataaaactcctctttctcctcccccttttctttcccctccagtcacttatcccctctcctccccctctcctccctacctaggagcagttctgccatccttttccattccttgttttatattcatctggagaaaactttgatattttaaggatacatcagccattttttggatttgagacccaacaagatagtaattcacctgtccagtcatgagaaaaaaactcttcaagttgataagtgttttgtttttttaaaaaacttcaatatttatttacttattttagtatatgagtatactgtagctgtatacatggttatgagccttcatgtagttgttgggaattagattttaggacctctgcctgctctcctctgccctgcttgatctaatctatttatggttgtaaataggtacactgtagctgtcttcagatgaaccagaagagggcatcagatcacattacaggtggttgtgaggcaccatgtgattgctgggatttgaactcagtaccttcagaagagcagtcagtactcttaccacctgagccatcttgccaggtccgtaagagttttctttaatcccataccaaataaagtgtaaaatagtttttgaccaaatattttcagtaggaagaaaggaaggaaggaaggaagggaagggaagggaaggaaggaaaggaaagaagaaatggaaggaagaaaagaatgaaaggaagggagatacagaaacgaattctgttagggtataaatgaaagagctgcaaaggaaataatgtgtagtcaattaaattcaaacaaaacacatgtctcccagccattctgcttctgggctgggcttgtgttgaagcagccattggtcccacgtgtgcatagaggggcaggtgcagcaaccaaatgagctccttgtacaactgagtgtaaaggggagggtgcagggaagggtctgatggcatttgtaaaaggaggaacctgaggatccaggcagagctgtctctaagggatacatccaccagaggtgcactgtggagtgcctacacagagacagaaagcacaatggtgggagggagggggagttccaagaccctatggttgccataggtttggagcaggtgagtggacagaaagactctgggatgggatggggccatctcaggaccccccttaccacccctgtgcaccctgaaatcacgacagtggccagggaaaggttccatagaaatagaaagattggagaaatttaggcacaggtggccctgaattatttccctaattaaggcaatcccccctctgtctctctgtctgtcaacctatagattacagtactgaggactttgcaacatgctagcaaaatgttctacaactgaggcacattccagccctcctgctactttttatgttgcaacagggtgtcactaaattgcccatgttggtcttcaactgtagtgcaggtgggtctggaacttgtgatcctcctgcctcagcctcctgagtagtctgggattccaggttgcaacactaggcccagcattgagcaactctttttcttcttcctgcttttcttccctttcccactcttggttagtatactaagaaataggttgcactgttacattctctctctctctctctctctctctctctctctctctctctctctcacacacacacacacacacacacacacccagcagaaacagggacatctaaattcaagggcagattggcctataaagccagttgcagtctaggcagggctacacagagagatcctgtctcaaataagcacatagctagatagataaataaatctctaaagcaaacataaccctgtgaacctgagttaggatccctgaataaaaagtcaggtttgactttattccctcaacttcagggctgtggggagtttagtcatagaggagaaattgataggacagatatcagagtcctcctttggtcctttcaggcatatatatacacacacacacacacacacacacacacatatatatatatatatacatacacacacacacacacacacacacacacacatatatatatatatatatagtacatgagtgcatacacacgcatgcacacatatacacccataaacactcatgcacagatacatacatatacccacatacacacacatgtacacacacatgtacacatacacactcatacacatatatacatacatacacatgcacacacacagcactcaaacatatacacccacaagcacactgtgtatgtcagagttcaaacagaccattgcttgcttggttgacagcaccttctcactgggctttttcaatcaccggcctccaagagaagcttatctttctgcaggcactgaggacctaggaacgcccttctgtgccaccttagggcaaaagcatccgcagacaacagcagttttgaatgaaactttatgtactacaaaagcaggtggcaagcctacttgggcccttgtcccttctttgctatcccccttctgtccaccctgacatcacaacacaagcagaagggacagactttgagaccataggatggcagccaggaaaatgactccccacagtgtgacctatcaaagtgtgtgtctttagctcaacaaaacaaaggcatggatcaggtagacaggaggacgctggtaggaagtctgtgacacactttgtggtttgtggtcatgcttggtatttggttatggcttttaggacctctcttactttgtgactttctgtgttggatcaggtttattcatcaataaatggtacatgaagttcaccatcaacagatcaatggatttcttttagtaaagccattgcccctggtgtctgtgtgtgttctgtgtgtttgcgtacatgactgttaatgtgtgcatgtgtgtgtgtttgtctgaaggtgttttgtgtgtttgtgtatgtacctgttcatgtgtgcatgtgtgtgtgtgtgtgtgtgtatacaagagtgtgcatttccatgtgtgtgtgtaaaacattcaggtgagctgccctgtgactttcttattcccttgataaaacatccttccctaaatctgcagtaccagtggcagccagccaaccccagtgatcctcctgcctcagctccccaagcactggtgcaaatataactgtagctttttacttgggtgctggtgattcaaacttaggggctcctgcctgtgcggcaggtgctcttatctatcaaggcatctccccactctctgtcttacttttctgaagagaaatttgataactctacaaatggccaagacaaagtccaaaggggcgatttagagcctgtgaaaggcactgacagcctggagacagtggctactttgtgggagatactctggggttaagcaggggtggtggaagagagtgccagagaggcagaagagcagcagacaccccaggccctgaggaaaaagacctaggagcagcaaacgagcaagtcaatagcatggctgatggaaccctgctcctaatgtactgctctctctattcccttcagaggagaacccatgtaactgtccacttgtggaagttaatcccccagcaggttattcccttcaaccccttgaattacgactttctacccacgacgtggaaattagagtccaggaacatatactgggcaacagatgggactcactggagattactggatcattcccaggcaagtgtttggtaccgcaagataaaagctgcagcaggtgtcccatgcttttcattctgacccacccttgggaggttcttctccctctcttccccacagtctagcccttggtccatttttagtggacagttgggaagtatggtatgagaaaacttttcttggggagatgagacaactcaagtacccagcatagactaggaacccacaacagatcaaagtatgcacagcacagaagcccaacttggtgaaccagtgaactttactgggttgacatacaggagcaccagtgtggggtcactcacaggaccagaaatgatccagttggatgcatcaccaaagcccaccacaaccttggtgacaaacagctcacaaaagctgggaccttgaagcacagtacccagcttacagtcagctaacaggtgggagagtgccctttccaggtgcctctgctggtctaaagctcctctaggcagcttggctggtcagcttcttctgggtccttttgtctgagtttgaggcttgactctgctgcttctggtagggagggacggtgtgattctgctcagtttcaggaagttcttgaagctactattgttgttgttatttccctgcttcagggtgttactgtattgcaacagagggagacagggagagggagaaaggagagagggagggtacagagagggaggaggggaagaaaagagaagatagagagagaatgcacatggggacacttctgcctactggtgtaggtgtgaaagtaatgcttccctctggctacagaatccagagaccaggtccccttttgctacttgtcccttcctgatgcctcaggtctgaatgggtcactagggctggctagacttgccttacaggaccctgatggctgtaccctctcctacagttgggtgacacagagcaactgatcctgatgctggtgttagggtgactgactgatggaagctgctgggaaggtttgtcagggtcagggaccacgaccctcactggtgaggtcagacagtgagccgctgactttccctctttataggaagacagtgctggcatctctcccaagctttcctggtgtaacacacacaggaagcacaggacatcacctgaatgacctaaagtcaccgctcacccctgggggaaaaaacctgaatgttctgtgttccacatcatagctaagcaaaagagcactgggtattggaattgtgggtcctatgatctcagagccctatgatcacactagaatccagtgactctggatctcaagtttaatcatacagtactgtgtgatcctggccacaggggacttacactgggcatctcctccaacttggtagcagctctctctggctaggtagagcttggaccatgggcagggctggctggagcccaagcctttggatctaggacccagttctgcagggatccactaatcagtgtctcatttgccttctacaggtcttgatagcctggctggctcctgctcctgtgccctcgggtagactttgcacccacccctctgtttgttcctcaagtttctagttctacagcaacttctgtcatggtcagacctactttttacatgatctactttctgtgagcctggtttgggtcctgaccgttctccctagctggagacaaactgcatcttctcttgcccactttgtgtctctggccctgaccttatagatggcttttcttaagacccctcctccattcaaactccttccacctgggcttctagtgcttccagtggtaggagatttgccccccatttaccttaggatgtctacaggtgcctctggaggaggtacagatagggactctatgccctgtgggcctttgacctatcactggcaagaagtttgcctggtgctgggtagaggacactctgtgctcagtagcttaaaggctcagtgtgagacttacagagaacatctggagtacttgtccaggtgctcagttccttggctccaggaagtgaatctgaccctaggcctctcccaccccaacttgtttttaactaattaaactttactgtttttactt

SEQ ID NO: 53 - DNA, 8196 bp, mouse
gtcattcccctgtctggccgcatttaagaaggcaggcaactggaaggcctcattactatggctgctgcaggtttttatcctccgaggctactcccacaagttctgatctccacaggaccgggcttctatgaggatgaacatcatagactgtggatggttgcaaaactggaaacttgttctcattcaccatattgcaacaaggtgagtttcagtgctccagacaggcgatcccccaactcttacccttgtgtgctaggctctgttgtgtgtgtgtgtgtgtgtttaggtaggtgctgttattatcgtgtcagaatagatgatacaggtaattgaatagaaacatgtccctataccctggagcttgcttggaggcttcacaggtccaactaatagacagatgaagagccctagcagtgaggacgctgttacagatagagagaacatgctgagaagggtttggtaagctgggctttgtgggatgagtaggagttctacaggtcagaaaaaaacaaaacaaaacaaaacaaacaaataaccaactctggtgccaggcagatttcctggaaggagcaaatgtaagtgccagcactactttccgcatttctgcaaatcataattcatctgctgctgttgctcccaggcttgagtggcctgaagaagtgtttgacagagaagggatccttacactaggagctgactacgggacttgggggaaagtcctaatgggttctgtgatctgcaggtgatgtagagacccacactgctggaaacctgctctggaccatggttttagtccacagcaggtccttgggggctaaaacactctctaatggaaggctctgggtatcaaaactgctccccagggttgtggaaaagactatgggccccctcccctggcctggaggtggagagaacttgggggccttaagtctctttggctgccagggttttcctgcaaccccaacccctgtcctaaccttttccagcccagtagcccttccctgcagctcccctgactccctgaatacttcagccattttggcaatggggctctcttggttcccccctcccctccctccttgacttgcaaggcccagatcaaggtcatgttcactctggactctcccaaatgtctgtttttgcctatgttcttccttttatctactatgaacctcctctttctcctcccccctcctcctccccatcctctcacttctccccctcctcctcagcctcctcacttctccccctccgccaaacccttctccccctcctcttcctgtacctcctccataccaaggagcatttatggcttcatttttcattccttgttttatattcatctaaagatgactttgatattttaatagacacctcagccatgtctttggtttgagacctaacaacagggtaattcacctgttcaattatatatatatatatatatatatatatatatatatatatatatatatatatcttcaagttgataagtgttttctttttttctcttttcattaaaaaaactccaatatttatttatttattttaatatatgagtacactgtactgtatacatggttgtgagccttcatatggttgtttggaattggattttaggacctctgcctgctctcatctgccctgcttgctctgatctatttattattataaataggtacacggtagctgtcttcagatgaaccagaataggacgtcagatctcattacaggtggttgtgaagcaccatttggttactgggatttgaacttagttcctttggaagagcagtcagtgctcttacccactgagctatcttgccagccccaataagagttttccttaaccctatactatacaaagtgtagaatagttttgaaccaaatattttcagtagaaacaaagaaagaaagaaaagaaggaaggaaggaaggaaggaaggaaggaaaaaagaaagaaagaaagaaaggaaggaaggaagaaaggaagaaaggaagaaaggacgaaaggacaaaaggaagaaaggacgaaaggaagaaaggacaaaaggaagaaaggacgaaaggaagaaaggacgaaaggaagaaaggatgaaaggaagaaagaaagaaagaaagaaagaaagaaagaaagaaaaaaaagagaaaatamnaaaaagagagagaaaaggnmggaagaaaagaagaacggaaggaagatccagaaaagaattctgttagggtataaatgaaatgagctgcaaaggaaataatgtgtaatcaattaaattcaaatagaatnacatgtctcccagccattcagctctgggctgggcttgtgttgaagcagcccattggtcccacgtgtgcatagaggggcaggtgcagcaaccaaatgagctccttgtacaactgagtgtaaaggggagggggcagggaagggtctgatggcatttgtaaaaggaggaacctgaggatgcaggcagagctgtctntaagggatacatccaccagaggtgcactgtggggtgccttcccagagacagaaagcacaatggtgggagggagggggagttccaagaccctatggttgccataggtttggagcaggtgagtggacagaaagactctgggatgggatggggccatctcaggaccccccttaccacctctgtgcaccctgaaatcaccacagtggccagggaaaggttccataggggtagaaagattggagaaatttaggcacaggtggccctgaattatttccctaattaaggcaatcccctctctgtctctctgtctgtcaacctatagattacagtaccgaggactttgcaacatgctagcaaaatgttctacaactgaggcacattccagccctcctgctactttttatgttgcaacagggtgtcactaaattgcccatgttggtcttcaactgtagtgcaggtgggtctggaacttgtgatcctcctgcctcagcctcctgagtagtctgggattccaggttgcaactctaggcccagcattgagcaactctttttcttcttcctgcttttcttccctttcccactcttggttagtatactaagaaataggttgcactgttacattctctctctctntctctctctctctctctctntctcwcacacacacacacacacacacacacacacacmcasccagaaacagggacatctaaattcaagggcagattggcctataaagccagttgcagtctaggcagggctacacagagagatcctgtctcaaataagcacatagctagatagataaataaatctctaaagcaaacataaccctgtgaacctgagttaggaccccagaataaaaagtcaggtgtgactttgtttgctgaacttcatggctgtggggagtttagtcacacaggaaaaagtgataggacagacatctgagtcctcctttggccttttcaggctggtatatatacatatactgtatgtgagtccacagacacacacccatatacatgcatacacacgtacatacacatgtacacacacacccatacacacacaagcacacacacatatgtgcacacatacacaccacacacacacatacacgaatacacacacaggcatacacacgtgcacatataccctcatacacacacacaggcacacacacatgcacacatacccacacatacacacccatatacacacatgcacatacacatacatgtgcacacatgtgcacacacagaaacacacacaaaaacatgcacacatgcacacacatatgcacacacaaatgcacgcacacacacacacacacatatgtggatcaattggctatatacagacagcttggtctccagtggagttgagagatgaaaacctgctgaaacccagtgttggtcattttcacctacatgggacagaaggcattcccccatctcctggacccctggctcctgtcgaagttaccacccccacaacccccacaggagaggctggtggctttcagtcttgtagacaatgccccaagcttctgccctttaggctagactcctcccagttacctagcaacaacaggataacaaggggctgtttggccccacctcactctctccctcttactctctaatctcttactctctaagctttacctctctctttaagctttcttagtctctagcctttattttctctctccttttctcctttatctcctcttggccatggtcagtctctctcttttaccttttctcaatccccctgcctttctacaataaagctctaaaagcatagactgtctctgttcatcaaggaccagagttaaaactctcgcctgcgtgggaacctctctctctcttcccactttctgtatctccaaggcctggtgctgctccggggcctctattctgttcttatctcctttgcactatccagcgtgggatacagaagactgagaacttagtatctgtgactgccccttgtccacccctggcagtgtggagtcagtggcttcacacagccagatgcccacccaggcccaagtggaaagcatccgttagtcctccctcatctacctgcccagagcacaggaactctggccaaacacaagctctctccctgccccctcattcccctacacccacagcaccacacacacacacacacacacacacacacacacggtgtatgtaagagttcacacagtctgttgctagcctggttgacagcaccttctcactgggctttttcaatcacaggcctccaagagatgcttatatttctgcaggcactgaggacctaggaaggctcccctgtgccaccttagggcaaaagcatccagagacaacagcagttttgaatgaaactttatgtactacaaaagcangtggcaagcctacttggggcccttgccccttctttgctatcccccttctgtccaccctgacatcacaacacaagcagaaaggacagactttgagaccataggatggcagccaggaaaatgactccccacaatgtgacctatcaaagtgtgtgtctttagcaaaacaaaggcatggatcaggaagacaagaggacgctggtaggaagtctgtgacacactttgtggtttgtggttatgattggtacttggttatggattttaggaactcttttactttgaggatttctgtgtgggctaagnawtnttttttacatctataaatggtgcatgaagttcaccatcaacagatcaatggatttcttgtttaaagccattgccccctggttctttgtgtgtgtgtatttctgtgtgtgtgtgtgtgtgtgtcctgtatgtttgtgtncatacctgttcatgtgtgcatgtgtgtgtgtatacaagtgtatgcatttgcatatgtgtgtgtgtgtgcgcgcgtgcacgcgcgtctgtgtaacattcaggtgagctgccctgtgactttcttattcccttgataaaacatccttccctaaatctgcagtaccagtggcaagccaacccaaccccagtggtccttcytgcctmagctcccccaagcactkggtgcaaatataactgwagcttttttacttgggtgntggtnnknnaatcttaggggctcctgcctgtgcggcaggtgctcttatctaccgaggcatctccccactctctgtcttacttttcagaagagatttttgaaaactcttaaaatggccaagacaaggtcagaaggggccatttagtgccatgaaaggcattacctggagcctggaggcagtggctactttgtgggagataccctagtgttgaacagggttggtggaagagagtgccagagaggcagaagagcagcagacaccccaggcnctgaggaagaagacctaggtgctggagacaaggctgtggttagsggtgttgaccacngcagcaaacaacccagtctcaggcatggaggatggaaccctgctcctaatgtaccgctctctctattcccttcagattgaaacatgtgtaactgttcacttgtggcagatgaccagatacccccaggagcctgctccctacaatcccatgaattacaactttctacccatgacgtggagattagcatccatgaacacataccggggaacagatgccatgcactggagattactgaatcattcccaggcaagtgtttgctacctcaagataaaagctgcagcaggtgtccagtgcttttcattctgacccacccgtgggaggttcttctccccctcttccccacagtcaagcccttggtccwtttwagtggatggttagggtctgtcgcatgacaaaccttttcttggggagatgagccaaactcaagtacccagcacagattgggaacccacaacagaccaaagtatgcacagcactgaagcccaacttggtgaaccagagaactttactgtggttacatacaggagcaccagagaggggtcactcacaggagcagaaatgagtcagttggctgcatcacgaaagcccaccacagacttgttgacagagagttcaaaaaagctgatgccttggatcacagtgcccagcctacggtcagctaacaggttggagagtgcctttgaggtgcctccgctggtccacgagccatttcaggcagcttggctgatcagcttcttcagagttcttttgtctgagtctttgaggcttgactctgctgcttcaggcagggaggggcagtgtgattctgctcagtttcaggggagttcttgaagctacttttgttgttgttacttccctgcttcaggaagtgactgctatgaaagaaagggtcagagggaaggagagagggggcagagagggagaggaagggggaaagatagaaggtagagagtgaatgcccatggggacacttctgcctactggtgtaggtgtgaaagtaacgcttccctctgactacacaatccagagaccaggtccccttttgctccttgtccctttggctgcctcaggtctgaatgggcaactagggctggctagatttgccttacaggaccctgatggctattccctctcctgtaggttggtgacacagtgcaactgatcctgatgctggagtaaaggtgactgactgatgggagctcctgggaaggtttgtcaggtcagggaccacgaccctcacaggtgaggtcacacaggtagccgctgaatttccctctttattggaagacagtgctggcatctccctccgagctttcctagtgtaacacacacaggaagcacaggacttctctgggatgacacaaagtcactgctcagacctgggtaaaaaaaatctgtatgctcagtgttccacatcatagctaaaccaaagagcttaattgggtgttggaattgtgggtcctatgacctcagagccctatgatcacactagaatccagtgactctggctctcaagtttaatcataaactgctgtgtgatcctggctccagaggacctacactaggcatctccaccagccgtgtagcagctctacctggctgtgtagagcttggaccatgggcagggctggctggagcccaagcctttggatctaggacccagttctgcagcgatccactaatcagtgtctcatttgccttctacaggtcttgatagcctggctggctcctgcccctgtgcccttggggtagactttgcacccacccctctgtttgttcctcaagtttctagttctacagcaacttctgtcatggtcagacctactttttacatgatctactttctatgagcctggcctatctctgtcctgaccgttctccctagctggagacaaactgcatcttctcttgcccactttgtgcctctggccctgaccttatagatggcttttcttaagacccctcctccattcaaactcctccacctgggcttctagtgccttgtgtggcaggagatttgtccccatttaccttaggatgtctacaggtgcctctggaggaggtacagatagggactctatgccctgtgggcctctgacctatcactggcaagaagtttgcctggtgctgggtagaggacactcagtgctcagtagcttgaaggcacagtgggagacttacagagaacatctggagtacttgtccaggtgctcagttccttggctccaggaagtgaatctgaccctaggcccctcccaccccaacttgttttaaagtaattaaactttactgtttttactt

SEQ ID NO: 54 - DNA, 4567 bp, mouse
gtcctgcccctctctgaacctcatttaagaaggcacgcaaccggacggcctcattactatggctgattcagttcattttccctggatgccattcccacctcgcttcctggtctgcacaagagatgacatctatgaggatgaaaatggtagacagtgggtagttgcaaaagtagaaacttctcgttcaccatatggcagcagggtgagtttcagggctccagtcaggttaccccctaactcttacctttgtgtgctaggctgtgtgtgtgtgtgtgtgtgtgtgtgtgtgtgtgtgtgtgtgtgtttgtgtttaggtaggtgctattattctcactcatcatatggcaacagggtgagtttcagggctccagacaggctatttcccaactcttacccttgtgagctagggtgtgtttacgtgtttgtgtgtgtgtgtgtgtgtgtgtgttgtgtgtaggtatgtacttttattactgtgtcagaaaatagctgatacaggtctctgaatagaaccttgtctatagctaggcctataccctggagcttgcttagaggcctcacaggtccaactaatacacagatgaagagccctagcagtaaggactccgttacagatagtcagagagaacatgctgagaaagctttgttaaaaaaaagaaagcaaaaaaaaaaaaaaaaaaaaaaaaagctgggctttgtggaatggtaggaattctacaggtcattaaaaaaacaaaacgaacaaataacaaactctggtgccaggcagattccctggaaggagcaaatgtgagtgccagcactagtttccgaattcatgtaaatcataattcatctgctgctgttgctcccaggcttgagtggcctgaagaagtgtttgacagacaagggatccttacactaggagctgactacaggacttgggggaagtcctaatgggttctgtgatctgcaggtgatgtagagacccacactgctggaaacctgctctggaccatggctttagtccacagcaggaccttgggggctaaaacactctctaatgaaggctctgggtatgaaaactgctccccagggttgtggaaaagactatgggctccctcctctgctctgggggtgaggagggagtggggatgttaggactgttttactgccaaggttttcctgcaccccatgccctgtcctaccttctccttctccagcccagcagcccttccctgcagtttccttgacttccacaatacctcagccattttgttgatgtaagttgcttggccccctctgccttgcccgttctttgctatcccccttctgtccaccctgacatcacaacacaagtagaagggacaaactttgcagaccttaggatggcagccaggaaaaaaactctccacagtgtgacctatcaatgtgtgtgtctttagcttaacaaaacaaaggtatggatcaggtagacaggaggacgctggtaggaagtctgtgacacactttgtgatttgtggtcatgcttggcacttggttatggtttttaggaacgcttttactttgtggctttctgtgtgggctcaggtttactttttgcttccatgaatggtacatgaagttcaccatcaacccttcagtggatttcttttggtaaagccattgccccctggtgtgtgtgcatgtgtctgtgtgtgtgtttattctgtgtgtttgtgtgcatgcctgttcatgtgtggatgtgtgttctgtgtgtttgtgtacatacttgatcatgtgtgcatatgtgtgtatgtatacaagtgtgtgcatttgcatgtgtgtgtgtctgtgtaacattcaggtgagctgccctgtgactttcttattcccttgataaaacatccttccctaaatctgcagttacagtggcagccagccaaccccagtggtcctcctgcctcagctccccaagcactggtgcaaatataactgtagctttttacttgggtgctggtgattcaatcttaggggctcctgcctgtgcggcaggtgctcttatctaccgaggcatctccccactctctgtcttacttttcagaagagaattttgaaaactctacaaatggccaagacagagccccaaggggccatttagtgccatgaaagacactgctagcctggagacagtggctactttgtgggacataccctggggttgagcagggttggtggaagagagtgccagagaggcagaagagcagcagacaccccaggccctgaggaagaagacctaggtgctggagacaaggctctggttagggatgttgaccactgcagcaaacaagctaggcccaggcatggaggatggaaccctgctcctaatgtaccgctctctctgttcccttcagattgaaacatgtataactgtgcacttgcagcatatgaccacaatcccccaggagcctactccccagcagcccattaataacaactctctccccacgatgtggagattagagtccatgaacacatacacgggaacagatgggacatactggagattactggatcattcccaggcaagtgttggttacctcaagataaaagctgcaggaggtatcccatgcttttcattctgacccacccctgggagggaggttctccctctcttcccccaactctagctcttggtccatttttagtggatgattggaacctatgatatgacaaaacttttatggggagatgagacaaactcaagtacctagcacagattgggaacccacaacagaccaaagtatgcacagaacagaagcccaacgtagtgaccagtgaactttactgggattacatacaggaacaccagtgaggggtcgctcacaggagctgaaatgactcagttgtgtgcatcaacaaagaacttaatgcatggtgactgacagttcacaaaaacagggaccttgaagcacagtgtccaggctacagtcagctaacaggtgggagagtgccctttccaggtgcctctgctggtctaaagcttttccaggcagctcggctggttggattcttctgggtccttttgtctgagtcttttaggcttgactctgctgcttctggtagggaggagcagtgtgattctgctcagtttcagggagttcttgaagctacttttgttgttgttacttccctgcttcaggaagtgactgctatgaaagaaagggagaaagggaaggagagagaaggtaaagtgagaatacacatggggacacttctgcctactggtgtaggtgtgaaagtaatgcttccctctggctacagaatcgagagaccaggtccccttttgctccttgtcccttcctgatgcctcaggtctgaatgggtcagtagggctggctagacttgccttacaggactctgatggctattccctctcttacagatgggcgacacattgcaactgatcctggacatagtaatatgcgaggttgactgaatgatggaagctgatgggaaggtttgtcagggtcagggaccacaaccgtcactggtgaggtcacacaggtagccgctgactttccctctttataggaagccagtgctggcatctctccctgagctttcctggtgtaacacacacaggaagcacaggacctcacggagatgacctaaagtcaccgttcagccctggggggaaaaacctgaatgttctgtgttccacatgatagctaagtaaaagagcactgggtgttggaattgtgggtcctatgatctcagagccctatgatcacactagaatccagtgactctggatctcaagtttaatcatacagtactgtgtgatcctggccacaggggacttacactgggcatctcctccaacttggtagcagctctctctggctaggtagagcttggaccatgggcagggctggctggagcccaagcctttggatctaggacccagttctgcagcgatccactaatcagtgtctcatttgccttctacaggtcttgatagcctggctggctcctgcccctgtgcccttggggtagactttgcacccacccctctgtttgttcctcaagtttctagttctacagcacctcctgccatgattagacctgctttttagattatctactttctgtgagcctggcctaactctgtcctgaccgttctccctagctggagacaaactgcatcttctcttgctcattttgtgcctctggccctgaccttatagatggcttttcttaagacccctcctccattcaaactcctccacctgggcttctagtgccttgtgtggcaggagatttgtccccatttaccttaggatgtctacaggtgcctctggaggaggtacagatagggactctatgccctgtgggcctctgagccatcactggcaagaagttcgactggtgctgggtagaggccactcagtgctcagtagcttgaaggcacagtgtgagacttacagagaacatctggagtacttgtccaggtgctcagttccttggctccaggaagtgaatctgaccctaggcccctcccaaccccaacttgtttttaactaattaaactttattgtttttactt

SEQ ID NO: 55 - DNA, 4570 bp, mouse
gtcattcccctgtctgaccgcatttaagaaggcaggcaaccagacagcttcattactatggctgattcagttcgttttccctgtatgccattcccaccttgcttcctggtctgcacaagagatgacatctatgaggatgaacatggtagacagtgggtagctgcaaaagtggaaacttcttctcattcaccatattgcagcaaggtgagtttcagggctccagtcaggttacctttgtgtgctaggctgggtgtgtgtgtgtgtgtgttgtgtgtgtgtgtgtgtgtgtgtgtgtgtgtgtgtgtgtgtttaggtaggtgctattattaccgtgtcagaaaatagctgatacaggtctctgaatagaaccttgtctttacctaggcccataccctggagctcgcttggaggcctcacaggtccaactaatagacagatgaagagccctagcagtgaggactctgtcaccaacagtcagagaaaacatgctgagaaagccctattaggctgggctttgtgggatgagtaggagttctacaggtcagaaaaaagaaagaaagaaagaaagaaagaaagaaagaaagaaagaaagaaagaaagaaagaaaatcaggtgcaaggcagattgagtccctggaaggagcaaatgtgagtgccagcactagtttccgaattcctgtaaatcataattcatctgctgctgttgctcccaggcttgagtggcctgaagaagtgtttgacagacaagggatccttacactaggagctgactacaggacttgggggaagtcctaatgggttctgtgatctgcaggtgatgtagagacccatgctgctggaaacctgctctggaccatggctttagtccacagcaggaccttgggggctaaaacactctctaatggaaggctctgggcatcaaaactgctccccagggttgtggaaaagactatgggctccctcctctgctctgggggtgaggagggagtggggatgttaggactgtttgactgccaaggttttcctgcaccccatgccctgtcctaccttctccttctcctgcccagcagcccttccctgcagttcccttgatttccacaatacctcaaccattttgttgatgtaagttgcttggccccctctgccttgccygttctttgctatcccccttctgtccaccctgacatcamaacacaagyagaagggacaaactttgcagaccttaggatggcagccaggaaaaaaactctccacagtgtgacctatcaatgtgtgtgtctttagcttaacaaaacaaaggyatggatcaggtagacaggaggasgctggtaggaagtctgtgacacactttgtgrtttgtggtcatgcttggcacttggttatggtttttaggaactcttttactttgtggctttctctgtgtgttcaggtttactttttgcatccatgaatggtacatgaagttcaccatcaacccttcagtggatttcttttggtgaagccactgccccctggtgtgtgtgtgtgtgttttgtgtgtttgtgtgcatgcctgttcatgtgtccatgtttgtgtgttcagtgtgtttgtgtacatacttgatcatgtgtgcatatgtgtgtatgtatacaagtgtgtgcatttgcatgtgtgtgtgtctgtgtaacattcagatgagctgccctgtgactttcttattcccttgataaaacatccttccctaaatctgcagttacagtggcagccagccaaccccagtggtcctcctgcctcagctccccaagcactggtgcaaatataactgtagctttttacttgggtgctggtgattcaatcttaggggctcctgcctgtgcggcaggtgctcttatctaccgaggcatctccccactctccatcttatttttcagaagagaattttgaaaactctacaaatggccaagacagagccccaaggggccatttagtgccatgaaaggcattacctggagcctggaggcagtggctactttgtgggagataccctggggttgagcaggggtggtggaagagagtgccagaggcagaagagcagcagacaccccaggccctgaggaaaaagacctaggtgctggagtcaaggttgtgtttaggggttttgtccactgtagcaaacaagccagtcccaggcttggaggatggaaccctgctcctaatgtaccgctctctctattcccttcagattgaaacctgtgtaactgtccacttgtggcagatgaccacactcttccaggagcctagtcccgactctctcaagactttcaactttctacccaggacatggagattagagtccaggaacacataccggggagcagatgccatgcactggagattagtgaatcattcccaggcaagtgtttgctacctcaagataaaagctgcagcaggcagctagcagggcatggtggcgcacacctttaatcccagcactcgggaggcagaggcaggcagatttctgagttcaaggccagcctggtctacagagtgagtgccaggacagtcaggactacacagagagaccctctctcaaaaaaccgaaagaaaaaaaaaagccgcagcaggtgtcccatgcttttcattttctcccaccccagggaaggaggttcttctccacccccttgacccaatgtggccttttgtccatttttagtggacagttggggtgtgtcatatgagaaaacttttttgggggagatgagacaaactcaagtacccagcacagattgggaacccacaacagaccaaagtatgcacagaacagaagcccaacgtagtgaccagtgaactttactggattatataccggaacactggtgagaggtcactcacaggagcagaaatgagtcagttggctgcatcaccaaagcccaccacagccttggtgacagacaattcacaaaaagctggtgccttgacgcacattgcccagcctacagtcagctaacagattggagagtgtcctttccaggtgcctcctgtggtctaaagctcttccaggcagcttggctggtcagcttcttctgggttcttttgtctgagtctttgaggcttgactctgctgcttctgacagggaggggcagtgtgattctgctcagtttcagggagttcttaaagctacttttgttgttgttacttccctgactcaggaagtgactgctatgaaagaaagggagaaagggaaggagagagaaggtaaagtgagaatacacatggggacacttctgcctactggtgtaggtgttaaagtaatgcttccctctggctacagaatccagagaccaggtccccttttgctcctgtcccttcctgatgcctcaggtctgaatgggtcactagggctagctagacttgccttacaggatcctgatggctgttccctttcctgcagttttatggcacagaggaactggtcctgatgctggattcaaggtaactgactaatggaagctgctgggaaggtttgtcagggtcagggaccacggccctcactggtgaggtcacacaggtagccgctgactttccctccttaaaggaagacaatgctggcatctctccctgagctttcctggtgtaacacacacaggaagtacaggacctcactgggatgacctaaagtcacctctcagccctgggtaaagaaaaaaaaaatctgtatgttcagtgttccacatcatagctaagccaaatagcttagttgagtgttggaattgtgagtcctacgatcacaatagaatccagtgactctggctgtcaagtttaatcatacagtgctgtgtgatcctggccacagggtacctacactgggcatctccccagcttggtagcagctctccctggctgtgtagagcttggaccatgggcagggctggctggagcccaagccttggatctaggacccagttctgcagcgatccactaatcagtgtctcatttgccttctacaggtcttgatagcctggctggctcctgcccctgtgcccttggggcagactttgcacccacctctctgcttgttcctcaagtttctagttctacaacacctcctgccatggtctttttacatgatcttctttctgtgagcctggcctatctgtggcttgacggttctccctagctggaggcaaactgcatcttctcttgctcactttgtgcctttgacactgaccttatagatggctcttaagacccctcttctcttcaaaatcctacacctgggcttctagtgccttgtgtggcaggagatttgtccccatttaccttaggagttctagaggtgcccctaaaggaggtacagatagagactctatgccgtgtgggcctctgagccatcactggcaagaagttcgcctggtgctgggtagaggacactcagtgctcagtagcttgaaggcacagtgggagacttacagagaacatctggaggacttgtccaggtgctcagttccttggctccaggaagtgatgctgaccctagacccctcccaaccccaacttgtttttaactaattaaactttactgtttttactt

SEQ ID NO: 56 - DNA, 4786 bp, mouse
gtcattcccctgtctgaccgcatttaagaaggcaggcaaccagacagcctcattactatggctgctgtgtctgttgatccccagaggccactcccagtcctcctggtctctgttagcctgggcatctatgaggatgaacatcatagagtgtggatagctgtaaacgtggaaacttctcattcatcacatggcaacagggtgagtttcagggctccagacaggttattccccaactcttatacctagtgggctagggtgtgtgtgtgtgtgtgtgtgtgtgtgtgtaggtaggtgttgttattaccatttcagaaaatagctgacacaggtctctgaataaaccttttctatacctaggcccataccctggagctctcttggaggcctcacaggtccaactaatacacaggtgcagagccctagtagtgaggactctgtcaccaacagtcagagaaaacatgctgagaaagctgggctttgtgggatgagtaggagttctacaggtcagaaaaaaaaaaaaaaaaaaaaagcaactctggtgccaggcagattccctggaaggagcaaatgtgagtgccagcactactttccccatttctgcaaatcataattcatctgctgctgttgctcccagacttgagtggcctgaagaagtgtttgacagcgaagggatccttacactaggagctgactacgggacttgggggaagtcctaatgggttctgtgatctgcaggtgatgtagagacccacactgctggaaacctgctctggaccatggctttagtccacagcaggtccttgggggctaaaacactctctaatgaaggctctgggtatcaaaactgctccccagtgtcctggaagagactatgggacacctcccctgtctgggggtggagaggaaataggggcgttaggtctctttgaatgctaggtttttcctgcagccccatcaccctatcctaaccttttctagcccagcagcccttccctgaagctcccctgactccctgaatacctcagccattttggcaatgggactctctttgtcccttatcctctccctcctcgcaggaaggaaggaaggaaggtagaaaggaaaaaaggaagggagggagggagggaggaaggaagggaaggaagggaaggaaggaaggaaaaaagaaagaaaagaagaaagaaacagaaaagaattttgttagggtataaatgaaagagctgcaaaggaaataatgtgtagtcaattaaattcaaacaaaggcacatgtctcccagccattctgcttctgggctgggcttgtgttgaagcagccattggtcccacgtgtgcatagaggggcaggtgcagcaaccaaatgagctccttgtacaactaagtgtaaaggagagggggcagggaagggtcggatggcatttgtaaaaggaggaacctgaggatccaggcagagctgtctctaagggatacatccaccagaggtgccctgtggagtgccttcacagagacagaaagcacaatggtgggagggagggggagttccaagaccctatggttgccataggtttagagcaggtgagtttacgaaaagactctgggatgggatgggggccatctcaggaccccccttaccacccccgtgcaccccgaaatcaccacagtggccaggaaaaggttccataggaatagaaagattggagaaatttaggcacaggtggccctgaattatttccctaattaagacaatttcccctctgtttgtctgtctctctatcaacctacagattacagtaacgaagacttttcaacatgctaccaaaatgttccacaactgaggcacattacagcctccttttactttttatgttgcaacagggtctcatgtgtgcatgtatgtgtgtctgtgtgtctctgtgtgtgttctgtatgtttgtgtacatacctgttcatatgtgcatatgtgtgtgtgtgtatacaagtgtgtgcatttgcatgtgtgtgtgtctgtgtaacattcaggtgagctgccctgtgactatcttattcccttgataaaacatccttccctaaatctgcagttacaatggcagccagccaatcccagtggtcctcctgcctcagctccccaagcactggtgcaaatataactgtagctttttacttgggtgctggtgattcaaacttaggggctcctgcctgtgcggcaggtgctcttatctaccgaggcatctccccactctctgtcttacttttcagaagagaattttgaaaactctacaaatggccaagacagagccccaaggggccatttagtgccatgaaagacactgctagcctggagacagtggctactttgtgggacataccctggggttgagcagggttggtagaagagagtgccagagaggcagaagagcagcagacaccccaggccctgaggaagaagacctaggtgctggagacaaggctctggttagggatgttgaccactgcagcaaacaagctaggcccaggatggaggatggaaccctgctcctaatgtaccgctctctctgttcccttcagattgaaacatgtgtaactgtgcacttgcagcatatgaccacactcccccaggagcctactccccagcagcccattaataacaactctctccccacgatgtggagattagagtccaggaacacatacacgggaacagatgggacatactggagattactggatcattcccaggcaagtgttgggtacctcaagataaaagctgcagcaggtgtccagtgcttttcattctgacccacccctgggagggaggttctccctctcttcccccaagtctagcccttggtccatttttagtggacgattggaacctatgatatgacaaaacttttatggggagatgagacaaactgaagtacccagcacagattgggaacccacaacagaccaaagtgtgcacagaacagaagcccaaattggtgaaccagtgaactttactgggattacatacaggaacaccagtgaggggtcgctcgcaggagctgaaatgactcagttgcgtgcatcaacaaagaacttaatggtattggtgacagacagttcacaaaaacggggaccttgaagcacagtgtccaggctacagtcagctaacaagttggagagtgccctttccaagtgcctctgctggtctaaagctcttccaggcagctggtcggattcttctgggtccttttgtctgagtctttgaggcttgactctgctgcttctgacagggagggacagtgtgattctgctcagtttcagggagttcttgaagctacttttgtagttgttacttccctgctttaggaagtgactgctatgaaagaaaaggtcagagggaaggagagaggaggcagagagggagagggaggaggggagagagaaggtagagagtgaatgcccatggggacacttctgcctactggtgtaggtgtgaaagtaatgcttccctctggctacagaatccagagaccaggtccccttttgctacttgtcccttcctgatgcctcaggtctgaatgggttactagggctggctagatttgccttacaggaccctgatggctattccctctcctacagatgggcgacacagtgcaactgaccctggacataataataggcgaggatgactgaatgatgggagctgctgggaaggtttgtcagggtcagggaccacgaccctcactggtgaggtcacacaggtagctgctgactttccctctttataggaagccagtgctggcatctctccctgagctttccttgtgtaacgcacacaggagcacaggacctcactgggatgacctaaagtcaccgctcagccctgggggaaaaacctgaatgttctgtgttccacatcatagctaagtaaaagagcactgtgtgttggaattgtgggtcctatgatctcagagtcctatgatcacactagaatccactgactgtgtctctcaagtttaatcataatctgctgtgtgatcctggctccagaggacctacactaggcatctccaccagccttgtagcagctctacctggctgtgtagagattggaccatgggcagggctggctggagcccaagcctttggatctaggacccagttctgcagcgatccactaatcagtgtctcatttgccttctacaggtcttgatagcctggctggctcctgcccctgtgccctcggggtagactttgcacccaaccctctgcttgttcctcaagtttctagttctaccacacctcctgccatgactagacctgctttttagattatctactttctgtgagccttgcctatctctgtcctgaccattctccctagctggagacaaactgcatcttctcttgctcactttgtgcctctgacactgaccttatagatggcttttcttaagacccctcctccattcaaactcctccacctgggcttctagtgccttgtgtggcaggagatttgtccccatttaccttaggatgtatacaggtgcctctggaggaggtacagatagggactctatgccctgtgggcctctgagccatcactggcaagaagttcgactggtgctgggtagaggacactacgtgctcagtagcttgaaggcacagtgggagacttacagagaatatctggagtacttgtccaggtgctcagttccttggctccaggaagtgaatctgaccctaggcccctcccaaccccaacttgtttttaactaattaaactttactgtttttactt

SEQ ID NO: 57 - protein, 116 aa, mouse
MAAAAFDPLGPLPVYLVSVRLGIYEDEHHRVWIVANVETSHSSHGNRRRTHVTVHLWKLIPQQVIPFNPLNYDFLPTTWKLESRNIYWATDGTHWRLLDHSQLGDTEQLILMLVLG

SEQ ID NO: 58 - protein, 129 aa, mouse
MAAAAFDPLGPLPVYLVSVRLGIYEDEHHRVWIVANVETSHSSHGNRRRTHVTVHLWKLIPQQVIPFNPLNYDFLPTTWKLESRNIYWATDGTHWRLLDHSQVLIAWLAPAPVPSGRLCTHPSVCSSSF

SEQ ID NO: 59 - protein, 117 aa, mouse
MAAAGFYPPRLLPQVLISTGPGFYEDEHHRLWMVAKLETCSHSPYCNKIETCVTVHLWQMTRYPQEPAPYNPMNYNFLPMTWRLASMNTYRGTDAMHWRLLNHSQVGDTVQLILMLE

SEQ ID NO: 60 - protein, 122 aa, mouse
MADSVHFPWMPFPPRFLVCTRDDIYEDENGRQWVVAKVETSRSPYGSRIETCITVHLQHMTTIPQEPTPQQPINNNSLPTMWRLESMNTYTGTDGTYWRLLDHSQMGDTLQLILDIVICEVD

SEQ ID NO: 61 - protein, 107 aa, mouse
MRLSGHRGLQWASLRFSGHRALQRASLKLSGHLIETCITVHLQHMTTIPQEPTPQQPINNNSLPTMWRLESMNTYTGTDGTYWRLLDHSQMGDTLQLILDIVICEVD

SEQ ID NO: 62 - protein, 112 aa, mouse
MPFPPCFLVCTRDDIYEDEHGRQWVAAKVETSSHSPYCSKIETCVTVHLWQMTTLFQEPSPDSLKTFNFLPRTWRLESRNTYRGADAMHWRLVNHSQFYGTEELVLMLDSRS

SEQ ID NO: 63 - protein, 121 aa, mouse
MAAVSVDPQRPLPVLLVSVSLGIYEDEHHRVWIAVNVETSHSSHGNRIETCVTVHLQHMTTLPQEPTPQQPINNNSLPTMWRLESRNTYTGTDGTYWRLLDHSQMGDTVQLTLDIIIGEDD

(19) United States
(12) Patent Application Publication
(10) Pub. No.: US 2005/0287530 A1
(43) Pub. Date: Dec. 29, 2005

(54) TCL-1b gene and protein and related methods and compositions

(76) Inventors: Carlo M. Croce, Philadelphia, PA (US); Yuri Pekarsky, Philadelphia, PA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/678,790

(22) Filed: Oct. 2, 2003

Related U.S. Application Data
(63) Continuation of application No. 09/526,329, filed on Mar. 15, 2000, now abandoned.
(60) Provisional application No. 60/124,714, filed on Mar. 15, 1999.

(51) Int. Cl. C12Q 1/68; C07H 21/04; A61K 39/395; C07K 14/82; C07K 16/30

(52) U.S. Cl. 435/6; 435/69.1; 435/320.1; 435/325; 530/350; 530/388.8; 424/155.1; 536/23.5

(57) ABSTRACT

The TCL1 gene family, located on the human chromosome at the 14q32.1 locus, are implicated in the development of T-cell malignancies. The present invention discloses the identification and characterization of new members of this gene family, the TCL-1b, TNG1 and TNG2 genes. The TCL-1b, TNG1 and TNG2 gene sequences are expressed at very low levels in normal bone marrow and peripheral lymphocytes, but are activated in T-cell leukemia and lymphoma by rearrangements of the 14q32.1 locus. The present invention relates to the identification of these chromosome 14 abnormalities, and methods for detecting and treating any T-cell malignancies that develop, as well as preventing the development of these T-cell malignancies.

### CROSS REFERENCE TO RELATED APPLICATIONS

This application claims priority, in part, under 35 USC § 119 based upon U.S. Provisional Patent Application No. 60/124,714 filed Mar. 15, 1999.

This invention was made in part with government support under Grant numbers CA39880 and CA51083 awarded by the National Institutes of Health. The government has certain rights in the invention.

### FIELD OF THE INVENTION

The present invention relates to the field of molecular biology, more particularly to the isolation and characterization of a third member of the TCL1 gene family, specifically TCL-1b, also activated by chromosomal rearrangements in T cell leukemias.

### BACKGROUND OF THE INVENTION

There is a cow association between particular chromosomal abnormalities, e.g., chromosomal translocations, inversions, and deletions, and certain types of malignancy indicating that such abnormalities may have a causative role in the cancer process. Chromosomal abnormalities may lead to gene fusion resulting in chimeric oncoproteins, such as is observed in the majority of the tumors involving the myeloid lineage. Alternatively, chromosomal abnormalities may lead to deregulation of protooncogenes by their juxtaposition to a regulatory element active in the hematopoietic cells, such as is observed in the translocation occurring in the lymphocytic lineage (Virgilio et al., 1993, Proc. Natl. Acad. Sci. USA, 90:9275-9279).

Non random chromosomal translocations are characteristic of most human hematopoietic malignancies (Haluska et al., 1987, Ann. Rev. Genet, 21:321-345) and may be involved in some solid tumors (Croce, 1987, Cell, 49:155-156). In B and T cells, chromosomal translocations and inversions often occur as a consequence of mistakes during the normal process of recombination of the genes for immunoglobulins (Ig) or T-cell receptors (TCR). These rearrangements juxtapose enhancer elements of the Ig or TCR genes to oncogenes whose expression is then deregulated (Croce, 1987, Cell, 41:155-156). In the majority of the cases; the rearrangements observed in lymphoid malignancies occur between two different chromosomes.

The TCL-1 locus on chromosome 14 band q32.1 is frequently involved in the chromosomal translocations and inversions with the T-cell receptor genes observed in several post-thymic types of T-cell leukemias and lymphomas, including T-prolymphocytic leukemias (T-PLL) (Brito-Babapulle and Catovsky, 1991, Cancer Genet. Cytogenet, 55:1-9), acute and chronic leukemias associated with the immunodeficiency syndrome ataxia-telangiectasia (AT) (Russo et al., 1988, Cell, 53:137-144; Russo et al., 1989, Proc. Natl. Acad. Sci. USA, 86:602-606), and adult T-cell leukemia (Virgilio et al., 1993, Proc. Natl. Acad. Sci. USA, 90:9275-9279).

The TCL1 oncogene on chromosome 14q32.1 is also involved in the development of chronic T-cell leukemia in humans (T-CLL) and is activated in these leukemias by, iuxtaposition to the T-cell receptor α/δ locus caused by chromosomal translocations, t(14;14)(q11;32), t(7;14)(q35;q32), or inversions inv(14)(q11;q32). Normally TCL1 expression is observed in early T-cell progenitors (CD4⁻CD8⁻CD3⁻) and lymphoid cells of the B-cell lineage: pre B-cells and immature IgM expressing B-cells. Introduction of a TCL1 transgene under the control of a lck promoter caused mature T-cell leukemia in mice. (Virgilio et al., 1998, Proc Natl Acad Sci USA, 95:3885-3889).

However, some cases of T-cell malignancies with abnormalities such as gene amplification at 14q32.1 did not show activation of the TCL1 expression, suggesting that perhaps an additional oncogene may be located in 14q32.1. The second member of the TCL1 gene family, MTCP1, is located at Xq28 and activated in rare cases of mature T-cell leukemia with a t(X;14)(q28;q11) translocation. The present invention involves the isolation and characterization of the third member of the TCL1 gene family, TCL1b, located at 14q32.1 and also activated by rearrangements at 14q32.1 in T-cell leukemias.

Rearrangements of the TCL-1 locus at chromosome 14q32.1 are unique, in that the other locus involved in these rearrangements, namely the TCR α/δ locus, is also on chromosome 14 at subband q11 (Croce et al., 1985, Science 227:1044-1047; Isobe et al., 1988, Proc Natl Acad Sci USA, 85:3933-3937). For this reason, the rearrangements observed cytogenetically are either chromosomal inversions, inv(14) (q11;q32), involving only one of the chromosomes 14 or translocations involving both. chromosomes 14 such as the t(14;14) (q11;q32), or more rarely, the t(7:14) (q35;q32) involving the TCR β locus at 7q35 (Isobe et al., 1988, Proc Natl Acad Sci USA, 85:3933-3937). Several of the breakpoints at 14q32.1 involved in these translocations have been cloned and characterized (Russo et al., 1988, Cell, 51:137-144; Baer, et al., 1987, Proc Natl Acad Sci, 84:9069-9073; Mengle-Gaw et al., 1987, EMBO 1:2273-2280; Bertness et al., 1990, Cancer Genet Cytogenet, 44:47-54).

The TCL-1 locus, a chromosomal region of approximately 350 kb as determined by placement of translocation breakpoints on the long range genomic map, has recently been cloned (Virgilio, et al., 1993, Proc Natl Acad Sci USA, 90:9275-9279). The involvement of such a large region in translocation events suggests that activation of the putative TCL-1 gene may occur from a distance of many kilobases, as previously observed for the BCL-1/CCNDI gene in mantle cell lymphoma (Tsujimoto, et al., 1984, Science 22,4:1403-1406; Rosenberg, et al., 1991, Proc Natl Acad Sci USA, 88:9638-9642; Withers, et al., 1991, Mol Cell Biol, 11:4846-4853; Motokura and Arnold, 1993, Genes Chrom & Cancer, 7:89-95) and the MYC oncogene in Burkitt lymphoma (Dalla-Favera, et al., 1982, Proc Natl Acad Sci USA, 79:7824-7827; Nishikura, et al., 1983; Proc Natl Acad Sci USA, 80:4822-4826) and in acute T-cell leukemia (Erikson, et al., 1986, Science, 232:884-886).

Introduction of a TCL1 transgene under the control of the T-cell specific lck promoter into mice causes T-cell proliferative disorder and, at the age of 15 months, T-cell leukemia (Virgilio, L., et al., 1998, Proc Natl Acad Sci USA, 95:3885-3889). Another member of the TCL1 gene family is the MTCP1 gene on chromosome Xq28. MTCP1 is also activated in rare cases of T-cell leukemia by a t(X;14)(q28;q11) translocation (Soulier, J., et al., 1994, Oncogene, 9:3565-3570). In rare cases of mature T-cell leukemias with chromosomal abnormalities at 14q32.1, activation of the TCL1 gene was not observed (Sakashita, K., et al., 1998, Leukemia, 12:970-971; Takizawa, J., et al., 1998, Jpn J Cancer Res, 89, 712-718). A second putative oncogene in this region was isolated, as described below, the TCL1b gene. This gene is located approximately 16 kb centromeric to TCL1 and shares 60% amino acid sequence similarity with TCL1.

The expression profiles of both genes are very similar. TCL1 and TCL1b are expressed at very low levels in normal bone marrow and peripheral blood lymphocytes (Virgilio, L., et al., 1994, Proc Natl Acad Sci USA, 91:12530-12534; Pekarsky, Y., et al., 1999, Proc Natl Acad Sci USA, 96:2949-2951), but at higher levels in T-cell lines containing rearrangements of the 14q32.1 region (Virgilio, L., et al., 1994, Proc Natl Acad Sci USA, 91:12530-12534; Pekarsky, Y., et al., 1999, Proc Natl Acad Sci USA, 96:2949-2951). Since genes in close proximity to TCL1 and TCL1b may also be activated in leukemias with rearrangements at 14q32.1, the chromosomal region bracketed by two previously published breakpoint cluster regions observed in T-cell neoplasias (Virgilio, L., et al., 1994, Proc Natl Acad Sci USA; 91:12530-12534; Virgilio, L., et al., 1993, Proc Natl Acad Sci USA, 90:9275-9279) at 14q32.1 was investigated for the presence of additional genes.

The murine Tcl1 locus was also examined in order to investigate the function of TCL1 and TCL1b. In the mouse the syntenic region of human chromosome 14q32 is the region of the murine chromosome 12 proximal to the immunoglobulin locus. The murine Tcl1 protein shows a 50% homology to the human Tcl1 (Narducci, M. G., et al., 1997, Oncogene, 15:919-926) and is expressed in fetal hematopoietic organs and in immature T and B-cells as well as in adult spleen and thymus (Narducci, M. G., et al., 1997, Oncogene, 15:919-926). In order to identify other members of the murine Tcl1 family the murine Tcl1 locus was also investigated for the presence of homologous genes.

There remains an unfulfilled need to fully isolate and characterize the other member of the TCL-1 gene family, TCL1b, and the genes located very closely to TCL1b and TCL1; TNG1 and TNG2. The identification of additional oncogenes that are associated with chromosomal abnormalities causing T-cell leukemias and lymphomas further expands the efficacy by which a diagnostic and therapeutic/prophylactic reagent will detect, treat, and prevent such disease states. The present invention fulfills this need by the identification and characterization of the TCL1b, TNG1 and TNG2 genes.

Citation of references herein above shall not be construed as an admission that such references are prior art to the present invention.

### SUMMARY OF THE INVENTION

The TCL1 gene family is implicated in the development of T-cell malignancies. The present invention discloses the identification and characterization of new members of this gene family, the TCL-1b, TNG1 and TNG2 genes. The present invention relates to the nucleotide sequences of TCL1b, TNG1 and TNG2, and amino acid sequences of their encoded Tcl1b, Tng1, and Tng2 proteins, respectively, as well as derivatives and analogs thereof, and antibodies thereto. The present invention further relates to nucleic acids hybridizable to or complementary to the foregoing nucleotide sequences, as well as equivalent nucleic acid sequences encoding a Tcl1b, Tng1 or Tng2 protein.

The present invention relates to expression vectors encoding a Tcl1b, Tng1 or Tng2 protein, derivative or analog thereof, as well as host cells containing the expression vectors encoding the Tcl1b, Tng1 or Tng2 protein, derivative or analog thereof.

The present invention further relates to the use of TCL1b, TNG1 and TNG2 genes and their encoded proteins as diagnostic and therapeutic tools for the detection and treatment of disease states associated with chromosomal abnormalities, specifically abnormalities at 14q32.1. In one embodiment of the present invention the use of nucleotide sequences of TCL-1b, TNG1 or TNG2 genes and amino acid sequences of their encoded Tcl-1b, Tng1, or Tng2 proteins, respectively, are used as diagnostic reagents or in the preparation of diagnostic agents useful in the detection of disease states, such as T-cell leukemias and lymphomas, associated with chromosomal abnormalities, in particular at 14q32.1, and/or increased levels of expression of the Tcl1b, Tng1 or Tng2 protein.

The invention further relates to the use of nucleotide sequences of TCL-1b, TNG1 or TNG2 genes and amino acid sequences of their encoded Tcl1b, Tng1 or Tng2 protein, respectively, as therapeutic/prophylactic agents in the treatment/prevention of disease states, such as T-cell leukemias, associated with chromosomal abnormalities, in particular at 14q32.1, and/or increased levels of expression of the Tcl1b, Tng1 or Tng2 protein.

The TCL-1b, TNG1 or TNG2 genes and Tcl1b, Tng1 or Tng2 protein sequences disclosed herein, and antibodies thereto, are used in assays to diagnose T-cell leukemias and lymphomas associated with chromosomal abnormalities, and/or increased expression of Tcl1b, Tng1 or Tng2 protein.

The Tcl1b, Tng1 or Tng2 protein, or derivatives or analogs thereof, disclosed herein, are used for the production of anti-Tcl1b, anti-Tng1 or anti-Tng2 antibodies, respectively, which antibodies are useful diagnostically in immunoassays for the detection or measurement of Tcl1b, Tng1 or Tng2 protein, respectively, in a patient sample.

Another aspect of the present invention relates to methods of treatment of diseases or conditions associated with chromosomal abnormalities and/or increased expression of Tcl1b, Tng1 or Tng2 proteins. Abnormalities of chromosome 14, such as inversions and translocations, particularly at 14q32.1, are associated with T-cell leukemias and lymphomas. TCL-1b, TNG1 or TNG2 gene sequences and their protein products are used therapeutically in the treatment of disease states associated with chromosome 14 abnormalities. Anti-Tcl1b, anti-Tng1 or anti-Tng2 antibodies are used therapeutically, for example, in neutralizing the activity of an overexpressed Tcl1b, Tng1 or Tng2 protein, respectively, associated with disease.

Oligonucleotide sequences, including antisense RNA and DNA molecules and ribozymes, designed to inhibit the transcription or translation of TCL-1b, TNG1 or TNG2 mRNA, are used therapeutically in the treatment of disease states associated with increased expression of Tcl1b, Tng1 or Tng2, respectively.

Proteins, peptides and organic molecules capable of modulating activity of Tcl1b, Tng1 or Tng2 are used therapeutically in the treatment of disease states associated with aberrant expression of Tcl1b, Tng1 or Tng2.

The present invention further relates to therapeutic compositions comprising Tcl1b, Tng1 or Tng2 proteins, derivatives or analogs thereof, antibodies thereto, nucleic acids encoding the Tcl1b, Tng1 or Tng2 proteins, derivatives or analogs, and TCL-1b, TNG1 or TNG2 antisense nucleic acid.

The present invention further relates to methods of production of the Tcl1b, Tng1 or Tng2 proteins, derivatives and analogs, such as, for example, by recombinant means.

### DESCRIPTION OF THE DRAWINGS

FIG. 1. Sequence comparison of Tcl1, Tcl-1b and Mtcp1. Identities are shown in black boxes, similarities are shown in shaded boxes. For Tcl1 and Mctp GenBank accession numbers are X82240 and Z24459, respectively.

FIG. 2 FIG. 2. Genomic organization of the TCL1 and TCL1b genes. Vertical arrows refer to cloned 14q32.1 breakpoints. Restriction sites are given for BssHII (B), ClaI (C), EagI (E), SfiI (F), KspI (K), MluI (M), NotI (N), NruI (R) and SalI (S). Solid boxes represent TCL1 and TCL1b exons.

FIG. 3. Northern analysis of the TCL1 and TCL1b genes. (A). Human immune system Northern blot. Lanes 1-6: spleen; lymph node; thymus; peripheral blood leukocyte; bone marrow; fetal liver. (B). Human cancer cell line Northern blot. Lanes 1-8: promyelocytic leukemia, HL-60; Hela cells; chronic myelogenous leukemia, K-562; T-lymphoblastic leukemia, MOLT-4; Burkitt's lymphoma Raji; colorectal adenocarcinoma, SW480; lung carcinoma, A549; melanoma, G361. (C). Lanes 1-6: Burkitt's lymphoma Raji; Burkitt's lymphoma Daudi; Burkitt's lymphoma CA-46; SupT11; bone marrow; placenta. (D). Lane 1: bone marrow; lanes 2-7, EBV transformed lymphoblastiod cell lines: Ado-1471; Ado-1476; Ado-1701; Ado-1727; Ado-2069; Ado-2199; lane 8: CA-46. (A-D). Top, TCL1b probe; middle, Tcl1 probe; bottom, actin probe.

FIG. 4. RT-PCR analysis of the TCL1 and TCL1b genes. (A). Normal human tissues. Lanes 1-23: heart; liver; brain; muscle; placenta; kidney; lung; pancreas; spleen; lymph node; thymus; tonsil; peripheral blood lymphocytes (PBL); fetal liver; fetal brain; fetal lung; fetal kidney; fetal heart; fetal skeletal muscle; fetal spleen; fetal thymus; negative control. (B) Lanes 1-4, T cell PLL samples: 3047; 3046; 3050; 3048. Lanes 5-6: bone marrow; PBL. (A-B). Top, TCL1b primers; middle, TCL1 primers; bottom, control G3PDH primers.

FIG. 5.: Genomic organization of human and mouse TCL1 loci. (A) Human TCL1 locus. Vertical arrows refer to cloned 14q32.1 breakpoints (1, 7). Restriction sites are given for BssHII (B), ClaI (C), EagI (E), SfiI (F), KspI (K), MluI (M), NotI (N), and SalI (S). Solid boxes represent exons of the four genes. (B) Striped boxes indicate translated parts of exons, white boxes indicate untranslated regions. Bold lines under the exons show various splicing products of TNG1, TNG2, and TCL1b genes. (C) Murine Tcl1 locus. Restriction sites and exons are indicated as in (A).

FIG. 6. RT-PCR analysis of TNG1 and TNG2 genes (A) Leukemia cell lines. Lanes 1-3: T-ALL cell lines: MOLT3; MOLT4; CEM. Lane 4: pre B-ALL cell line 697. Lane 5: T-ALL cell line SupT11. Lane 6-8: Burkitt's lymphoma cell lines CA-46; Raji; Daudi. Lanes 9-10: bone marrow; peripheral blood lymphocytes (PBL). First panel, TCL1 primers; second panel, TCL1b primers; third panel, TNG1 primers; fourth panel, TNG2 primers; bottom, control G3PDH primers (B). Normal human tissues. Lanes I-23: heart; liver; brain; muscle; placenta; kidney; lung; pancreas; spleen; lymph node; thymus; tonsil; PBL; fetal liver; fetal brain; fetal lung; fetal kidney; fetal heart; fetal skeletal muscle; fetal spleen; fetal thymus; negative control. (C) Lanes 1-4, T cell PLL samples: 3047; 3046; 3050; 3048. Lanes 5-6: bone marrow; PBL. (B-C). Top, TNG1 primers; middle, TNG2 primers; bottom, control G3PDH primers.

FIG. 7. Northern analysis of TNG1 and TNG2 genes. Lanes 1-3: Burkitt's lymphomas Raji; Daudi; CA-46; Lane 4: T-ALL SupT11; Lanes 5-6: bone marrow; placenta. Top, TNG1 probe; middle, TNG2 probe; bottom, actin probe. Each lane contains 3 μg of polyA+ RNA.

FIG. 8. RT-PCR analysis of murine Tcl1b genes. (A-B) Nested PCR, except β-actin. The panels are in the same order. (A) Normal mouse tissues. Lanes 1-13: heart; brain; spleen; lung; liver; sceletal muscle; kidney; testis; 7 day embryo; 11-day embryo; 15-day embryo; 17-day embryo; negative control. (B) Lymphoid cell lines. Lanes 1-5: B-cell lines NFS-5; NFS-70; WEHI-279; MOPC-31C; MPC-11. Lanes 6-7: T-cell lines S49.1; BW5147. Lane 8-9: ES cells; negative control. (C) Single round of RCR. Lanes 1-4: ES cells, mouse oocytes; 2-cell embryos; negative control.

FIG. 9. Sequence comparison of human and murine Tcl1, Tcl1b and Mtcp1 proteins. Identities are shown in black boxes, similarities are indicated by shaded boxes. *mark the conserved residues of the inner hydrophobic core.

FIG. 10. Location of the insertion in human and murine Tcl1b proteins: A side view of human Tcl1 is shown in green. The Tcl-1b insert into the C-D loop is shown in blue.

### DESCRIPTION OF THE INVENTION

Methods

Cell Lines.

Cell lines, except EBV transformed lymphoblastoid cell lines, were obtained from ATCC (Rockville, Md.) and grown in RPMI media with 10% fetal bovine serum. Lymphoblastoid cell lines were made from peripheral blood lymphocytes of patients with Alzheimer's disease by transformation with Epstein-Barr virus (EBV) as previously reported (Ounanian, A., et al., 1992, Mech Ageing Dev, 63:105-116).

Human leukemia cell lines MOLT 3, MOLT 4, CEM, and SupT11 (T-cell leukemias) and 697 (pre B-cell leukemia) and CA-46, Raji, and Daudi (Burkitt's lymphomas) were obtained from American Type Culture Collection (Manassas, Va.) Mouse lymphatic cell lines NFS-70 C-10 (pro B-cells), NFS-5 C-1 and WEHI-279 (pre B-cells), MOPC-31C and MPC-11 (plasma cells), and S49.1 and BW 5147 (thymocytes) were also purchased from American Type Culture Collection (Manassas, Va.). All cell lines were grown in RPMI 1640 medium with 10% fetal bovine serum.

Northern, Rapid Amplification of cDNA Ends (RACE) and Reverse Transcripton-PCR (RT-PCR) Analysis.

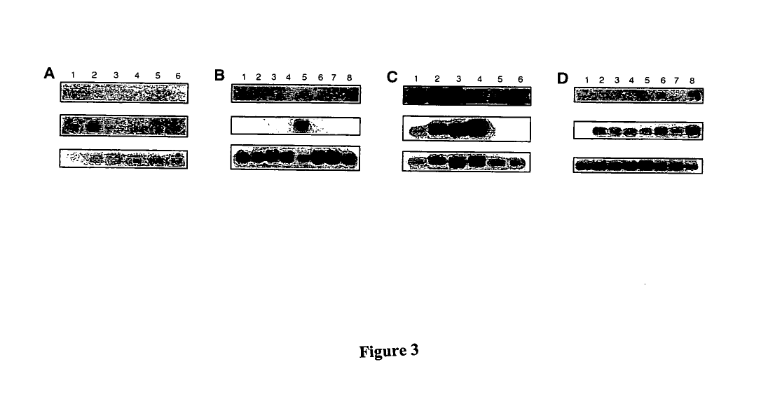

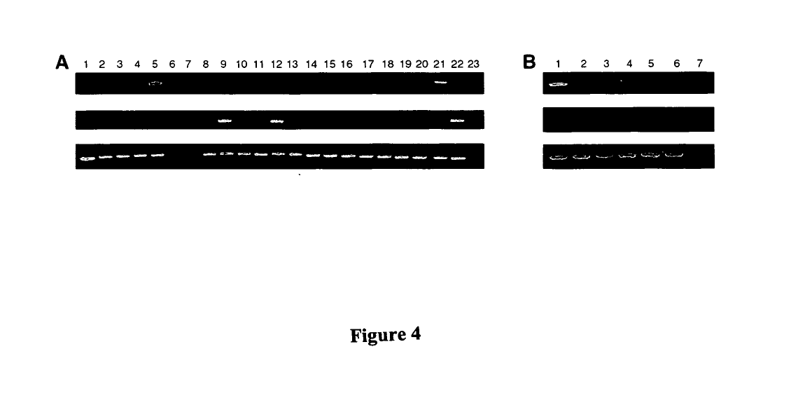

These experiments were carried out as previously described (Pekarsky, Y, et al., 1998, Proc Natl Acad Sci USA, 95:8744-8749) with the following exceptions. Human bone marrow and placenta mRNAs, human immune system and human cancer cell line Northern blots were purchased from Clontech (Pato Alto, Calif.). Each line on FIGS. 3C and D contains 3 mg PolyA+ RNA. PCR shown on FIG. 4A was carried out for 25-35 cycles using Multiple Tissue cDNA Panels (Clontech) and manufacturer's protocol. Primers were: top panel, TC1 GGCAGCTCTACCCCGGGATGAA, (SEQ. ID. NO: 1); and TC39 ACAGACCTGAGTGGGACAGGA, (SEQ. ID. NO: 2); middle panel, TCLB TCCTCCTTGGCAGGAGTGGTA, (SEQ. ID. NO: 3); and TCLC CAGTTACGGGTGCTCTTGCGT, (SEQ. ID. NO: 4); lower panel, control 3′ and 5′ RACE G3PDH primers (Clontech). FIG. 4B, middle and bottom panels, primers were the same as above. FIG. 4B, top panel. PCR was carried out for 22 cycles with primers TC8 ATGGCCTCCGAAGCTTCTGTG, (SEQ. ID. NO: 5), and TC39. 0.1 ml of the reaction was used for the second PCR with nested primers TC10 TGGTCGTGCGGTTCAATCCCT, (SEQ. ID. NO: 6); and TC5 AATCTGGCCATGGTCTGCTATTTC, (SEQ. ID. NO: 7); for 15 cycles. RACE primers were: TC1 (for 3′ RACE) and TC5 (for 5′ RACE).

Mouse and human tissue cDNAs for RT-PCR and RACE experiments were purchased from Clontech (Palo Alto, Calif.). Mouse egg and 2 cell embryo cDNA libraries for embryonic expression studies in mouse were previously described (Rothstein, J. L., et al., 1992, Genes Dev, 6:1190-1201). The DNAs from these libraries were diluted to the same concentration of cDNA as in mouse tissue samples. RNA extractions and reverse transcriptions from human and mouse cell lines and mouse embyonic stem cells were performed using Trizol™ reagent (Gibco BRL, Grand Island, N.Y.). 2 μg of total RNA were transcribed into cDNA in a total volume of 20 μl using SuperScript™ reverse transcription kit (Gibco BRL, Grand Island, N.Y.) according to the manufacturers instructions. 1 μl of this reaction was used for PCR. RT-PCR for TNG1 was carried out with primers 1A TGCATCCCTCCAGCCAAGGAT, (SEQ. ID. NO: 8); and 4A TGGCCTGCAGAGGCTCTCAAG, (SEQ. ID. NO: 9); for 25-35 cycles. For TNG2 primers 3B GTGCCTGTCTCATTCGCCTCTG, (SEQ. ID. NO: 10); and 8B AGTGGGCACATGTTACAGCATTC, (SEQ. ID. NO: 11); were used for the first round of 25 cycles and primers 4B GCATCCAGGACTGTGCCAGCA, (SEQ. ID. NO: 12); and 9B TTCTGTTAGCCTTGCTGTCCGT, (SEQ. ID. NO: 13); were used to amplify 0.1 μl of the first reaction in a nested PCR of 20 cycles. PCR conditions were 94° C. denaturation for 30 sec, 54 to 62° C. annealing for 30 sec and 72° C. extension for 30 sec. TCL1, TCL1b and, as control, G3PDH were amplified as described previously (6). RACE analysis was carried out in a nested reaction with 30 cycles in the first round and 25 in the second. The primers were: TNG1: 1A and 2A TTGAACCCAGGTCTCGTCTGAC, nested, (SEQ. ID. NO: 14); for 3′ RACE and 3A AACGTAGGATGTGCACAGAGCA, (SEQ. ID. NO: 15); and 4A (nested) for 5′ RACE and TNG2: 3B and 4B (nested) for 3′ RACE and 8B and 9B (nested) for 5′ RACE together with primers AP1 and AP2 supplied by Clontech (Palo Alto, Calif.) fitting to the adapters of the cDNA. The murine Tcl1b genes were amplified using the respective R reverse: 1R: GAGAACGGTCAGGACCCAAACC, (SEQ. ID. NO: 16); 2R: CAGGCTATCAAGACCTTTACTC, (SEQ. ID. NO: 17); 3/5R: TCAACCTCGCATATTACTATGTC, (SEQ. ID. NO: 18); 4R: CAAAGGCACAAAGTGAGCAAGAG, (SEQ. ID. NO: 19); and F forward: 1F: AATGTGGAAACTTCTCACTCAT, (SEQ. ID. NO: 20); 2F: ACTGGAAACTTGTTCTCATTCAC, (SEQ. ID. NO: 21); 3/5F: CACTTGCAGCATATGACCACAAT, (SEQ. ID. NO: 22); 4F: CCTGGTCTGCACAAGAGATGA, (SEQ. ID. NO: 23); primers for 28 cycles. Subsequently the respective R and FN forward nested: 1FN: CTGTCCACTTGTGGAAGTTAAT, (SEQ. ID. NO: 24); 2FN: CACTTGTGGCAGATGACCAGATA, (SEQ. ID. NO: 25); 3/5FN: CCAGGAGCCTACTCCCCAGCAG, (SEQ. ID. NO: 26); 4FN: GTGGCAGATGACCACACTCTT, (SEQ. ID. NO: 27); primers were used in a seminested PCR for 25 cycles to amplify 1 μl of the first reaction. PCR conditions were the same as described for human tissues. Due to the similarity of mouse Tcl1b genes it was difficult to find specific primers for each of them. Subsequently Tcl1b3 and Tcl1b5 were amplified with the same primers and sequenced to verify the expressed gene. However, in the case of embryonic tissue, unique forward primers were used to analyze the expression of Tcl1b3 and Tcl1b5 separately. The expression of both alternative first exons of Tcl1b3 was verified using the primers 3F horn homologous exon 1: CATTACTATGGCTGATTCAGTTC, (SEQ. ID. NO: 28); and 3F alt alternative exon 1: GGAATGAGACTCTCAGGGCAC, (SEQ. ID. NO: 29); instead of 3/5F. RT-PCR for Tcl1 was carried out similarly with primers Tcl1R, CCTGGGCAAGGCAGACAGGAGC, (SEQ. ID. NO: 30); and TCL1F, TGCTTCTTGCTCTTATCGGATG, (SEQ. ID: NO: 31); followed by a nested PCR using primers Tcl1RN, TTCATCGTTGGACTCCGAGTC, (SEQ. ID. NO: 32); and Tcl1FN, AATTCCAGGTGATCTTGCGCC, (SEQ. ID. NO: 33). The quality of the cDNA was verified by 25 cycles of β-actin RT-PCR using primers actR, GTACCACCAGACAGCACTGTG, (SEQ. ID. NO: 34); and actF, GACCCAGATCATGTTTGAGACC, (SEQ. ID. NO: 35); RACE analysis from mouse tissues was performed as described above for human tissues. The specific primers were: allR, AAGCCATCTATAAGGTCAGG, (SEQ. ID. NO: 36); for the first step and the respective R primers for the nested step of 5′ RACE and the respective F (first) and FN (nested) primers for 3′ RACE.

Pulsed-Field Gel Electrophoresis (PFGE) Analysis and Chromosomal Localization.

PFGE analysis was performed as described (Pekarsky, Y., et al., 1998, Proc Natl Acad Sci USA, 95:8744-8749), except pulse time was 1-6 second for 11 hours. Chromosomal localization of the TCL1b gene was carried out using GeneBridge 4 radiation hybrid mapping panel (Research Genetics, Huntsville, Ala.) according to the manufacturer's protocol. Primers were TC1 and TC4, TGCTAGGACCAGCTGCTCCATAGA, (SEQ. ID. NO: 37).

Sequencing.

Products from RACE and RT-PCR experiments were cut and extracted from agarose gels using a QIAquick gel extraction kit (Qiagen, Valencia, Calif.) according to the manufacturer's instructions. Subsequently they were sequenced using an automated sequencer model 377 (Perkin Elmer, Foster City, Calif.). A human Bacterial Artificial Chromosome library (BAC) (277A8) was partially digested with Sau3A and TSP509I and cloned into a pUC18 vector using standard methods. 100 random clones were isolated and sequenced from both ends using a 377 automated sequencer. The DNA sequences were compared to the expressed sequence tag (EST) database. The mouse BAC 452-I24 was sequenced and analyzed as described previously (Inoue, H., et al., 1997, Proc Natl Acad Sci USA, 94:14584-14589). EST clones were purchased from Research Genetics (Huntsville, Ala.) and sequenced.

Northern Blot and Pulse-Field Gel Electrophoresis (PFGE) for TNG Gene

Total RNA for Northern blot experiments was isolated as described above. PolyA+ RNA isolation, Northern blotting and hybridization was performed as previously described (Hallas, C., et al., 1999, Clin Cancer Res, In press). TNG1 and TNG2 probes were generated by RT-PCR. PFGE analysis was performed as described⁷ using BAC (277A8) DNA and TNG1, TNG2, TCL1, and TCL1b probes.

Protein Structure.

A computer model was created for the human and murine Tcl1b proteins based on their similarity to Tcl1. The atomic coordinates for human TCL1 are derived from the crystal structure (Hoh, F., et al., 1998, Structure, 6:147-155). The initial sequence alignment was generated by maximizing the correlation between the sequences. Modeling and analysis were done using InsightII (Biosym, San Diego, Calif.).

Results

Identification of the TCL1b Gene.

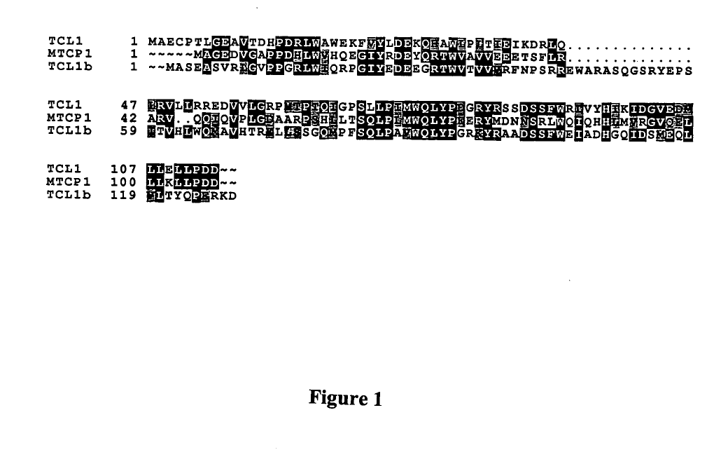

In some mature T-cell leukemias with chromosomal abnormalities at 14q32.1, activation of the TCL1 gene at 14q32.1 was not observed (Takizawa, J., et al., 1998, Jpn J Cancer Res, 89:712-718; Sakashita, et al., 1998, Leukemia, 12:970-971). To investigate the possibility that other, unknown TCL1 family member(s) may be involved, we searched the EST database for sequences homologous to the TCL1 and MTCP1 gene products. A single EST (accession number AA689513) was found to be homologous, but not an exact match to both genes. Thus, a ˜1.2 kb full length cDNA (SEQ. ID. NO: 38) was isolated using 5′ and 3′ RACE procedure and human testis mRNA as a cDNA source. The 1.2 kb TCL1b cDNA encodes a 14 kDa protein of 128 amino acids (SEQ. ID. NO: 39) (FIG. 1). It contains a starting ATG codon at position 28 within a perfect Kozak consensus sequence. The Tcl1b protein has a 14 amino acid insertion compared to the Tcl1 and Mtcp1 proteins (FIG. 1); it is 30% identical and 60% similar to Tcl1, and 36% identical and 63% similar to Mtcp1 (FIG. 1).

A radiation hybrid mapping panel (GeneBridge 4) was used to determine the chromosomal localization of the human TCL1b gene. By analysis of PCR data at the MIT database (http://www-genome.wi.mit.edu), the TCL1b gene was localized to 3.05 cR from the marker D14S265, at 14q32. A TCL1b pseudogene and localized it to 5q12-5q13. The TCL1b pseudogene does not have the initiating ATG or introns and has a stop codon in the middle of the open reading frame.

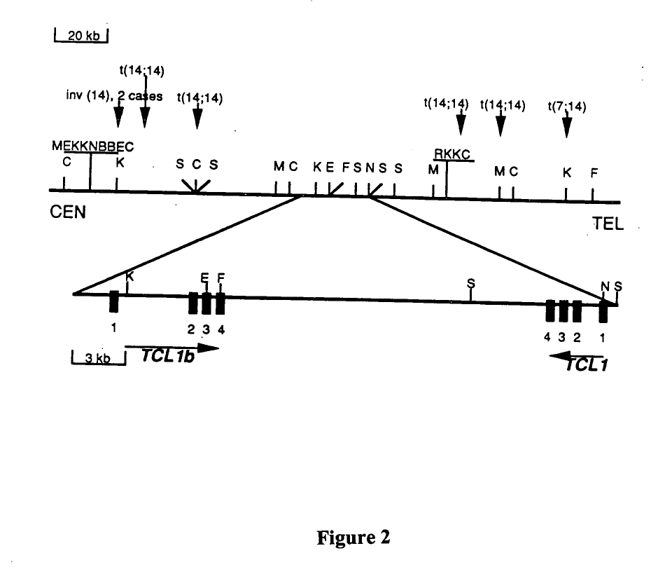

TCL1 and TCL1b are both located at 14q32, therefore, a determination was made as to whether TCL1 and TCL1b are physically linked. The human bacterial artificial chromosome (BAC) library and found several BAC clones containing TCL1 and TCL1b. The TCL1b gene (SEQ. ID. NO: 40) is 6.5 kb in size and contains 4 exons of 189, 171, 69 and 697 bp respectively (FIG. 2), but only the first three exons are coding. Pulsed field analysis of the positive BAC clone with both probes revealed that the TCL1 and TCL1b genes have opposite directions of transcription and are separated only by 16 kb (FIG. 2). Both genes are located in the ˜160 kb region between previously published two sets of breakpoints observed in T-cell acute lymphoblastic leukemia (ALL) cases with translocations or inversions at 14q32.1 (Virgilio, L., et al., 1994, Proc Natl Acad Sci USA, 91:12530-12534; Virgilio, L., et al., 1993, Proc Natl Acad Sci USA, 90:9275-9279).

Expression of TCL1b Gene and its Activation in T-Cell Malignancies.

Because of the similarities between the TCL1 and TCL1b genes in their structure, sequence, and location, it seemed possible that they would exhibit similar expression patterns. To verify this, we carried out a series of Northern and RT-PCR experiments (FIGS. 3 and 4). Northern analysis in normal tissues was mostly negative for TCL1b (FIG. 3A), except that the 1.2 kb transcript was detected after several days exposure in testis and placenta (FIG. 3C). The TCL1 gene expression, however, was detected in most hematopoietic tissues after several days exposure (FIG. 3A). Semiquantitative RT-PCR analysis (FIG. 4A) revealed that both TCL1 and TCL1b genes are expressed in spleen, tonsil, fetal liver, fetal kidney, and fetal thymus. However, the TCL1b gene is expressed in wider variety of tissues including placenta, kidney and fetal spleen (FIG. 4A). Northern analysis of commercial human cancer cell lines showed that TCL1 and TCL1b are expressed in only the Raji Burkitt lymphoma cell line (FIG. 3B), although TCL1 was expressed at a much higher level (FIG. 3B).

The TCL1 and TCL1b genes have similar transcription patterns and are physically linked. Therefore, a determination as whether the TCL1b gene could also be activated by rearrangements in 14q32 was made. FIGS. 3C and 3D show the activation of the TCL1b gene in a T-leukemia cell line with a translocation at 14q32.1 (SupT11) compared with the normal bone marrow and with EBV transformed lymphoblastiod B cell lines expressing TCL1. (FIGS. 3C and 3D, middle panels). Since TCL1 and TCL1b are normally not expressed in post-thymic T-cells and post-thymic T-cell leukemias lacking 14q32.1 abnormalities (for example, in T-ALL MOLT4 with no abnormalities at 14q32.1, FIG. 3B, lane 4), the expression of TCL1 and TCL1b in SupT11 cells carrying a t(14;14)(q11;q32,1) translocation indicates that juxtaposition of TCL1 and TCL1b to the α/δ locus of the T-cell receptor deregulates both genes.

To further investigate TCL1b expression, four T-cell leukemias and six EBV transformed lymphoblastoid cell lines with elevated levels of TCL1 were analyzed. FIG. 4B shows the activation of the TCL1b expression in one leukemic sample from a patient with T-cell prolymphocycic leukemia. Human T-cell prolymphocytic leukemias carry the 14q32.1 translocation or inversion and overexpress TCL1 (Virgilio, L., et al., 1994, Proc Natl Acad Sci USA, 91:12530-12534; Narducci, M. G., et al., 1997, Cancer Res, 57:5452-5456). The TCL1b gene was also expressed in two out of six EBV transformed lymphoblastoid B cell lines (FIG. 3D, upper panel, lanes 2-7).

The Human TCL1 Locus.

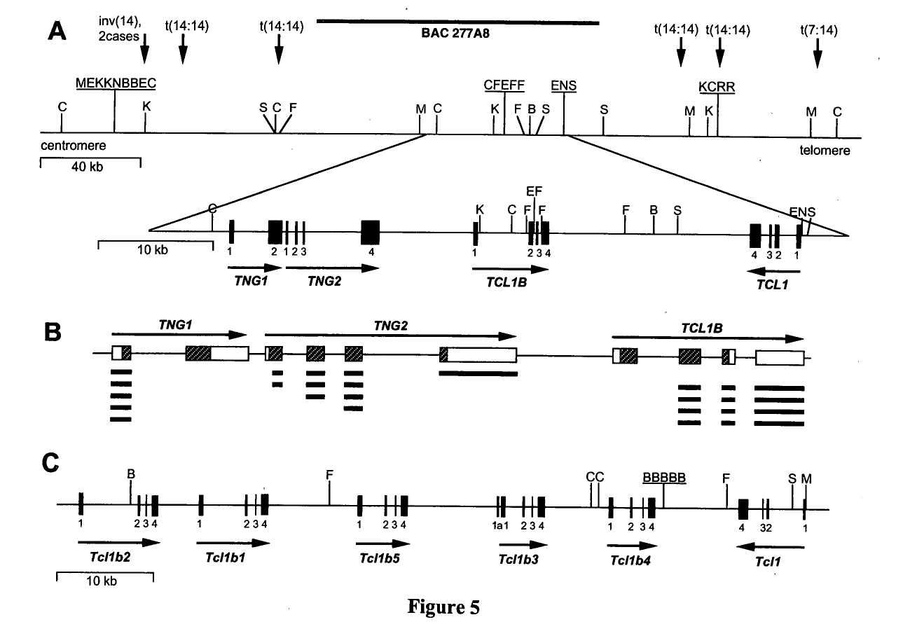

The TCL1 and TCL1b genes are both located on chromosome 14q32.1 within a ˜160 kb region between two previously published breakpoint cluster regions observed in T-cell neoplasms (Virgilio, L., et al., 1994, Proc Natl Acad Sci USA, 91:12530-12534; Virgilio, L., et al., 1993, Proc Natl Acad Sci USA, 90:9275-9279). Both genes are activated by translocations and inversions involving 14q32.1 (Pekarsky, Y., et al., 1999, Proc Natl Acad Sci USA, 96:2949-2951). To investigate whether other, unknown genes within this region are also activated by the same rearrangements a previously isolated bacterial artificial chromosome library (BAC) of 110 kb (277A8, ref. 6) covering the majority of this region was analyzed. This BAC was partially digested with the restriction enzymes Sau3A and TSP509I and cloned into a pUC18 vector. 100 clones (the equivalent of the length of the BAC) were picked randomly and sequenced from both sides. These sequences were compared to the expressed sequence tag (EST) database and two different sets of ESTs homologous to the BAC sequences were found. Two full length cDNAs using 3′ and 5′ RACE and RT-PCR of cDNA from human testis, peripheral blood lymphocytes, and the Burkitt's lymphoma cell line Raji were isolated using primers made from the different ESTs. The 1.5 kb cDNA of the TCL1 neighboring gene 1 (TNG1) (SEQ. ID. NO: 41) contains an open reading frame coding for a protein of 141 amino acids (SEQ. ID. NO: 42) with the start codon ATG at position 161. The 2 kb cDNA of TCL1 neighboring gene 2 (TNG2) (SEQ. ID. NO: 43) encodes a shorter protein of 110 amino acids (SEQ. ID. NO: 44) with the start codon at position 36. Both genes do not show homology to any known genes found in the database. Relative positions of the genes and their distances from each other were determined by Southern hybridization and pulse field Southern analysis. TNG2 is located 8 kb centromeric of TCL1b and TNG1 is only 118 bp centromeric of TNG2. TNG1, TNG2 and TCL1b have the same transcriptional orientation, opposite to TCL1 (FIG. 5A). The TNG1 gene is 4.5 kb (SEQ. ID. NO: 45) in size and contains only two exons of 215 and 1239 bp. The TNG2 gene has a size of 8.6 kb (SEQ. ID. NO: 46) containing four exons of 134, 136, 157, and 1651 bp, all of which are coding. RT-PCR and RACE experiments revealed several alternatively spliced RNAs linking various exons of TNG1 and TNG2 to exon 2 of TCL1b (FIG. 5B). Only one of these RNAs, linking the exon 1 of TNG1 in frame to the second exon of TCL1b, contains a new open reading frame encoding a TCL1b protein with an alternative N-terminal end.

The Murine Tcl1 Locus.

In order to identify the murine Tcl1b gene the murine expressed sequence tag (EST) database was searched for sequences homologous to human TCL1b. Three sets of ESTs were found that were very similar, but not identical, to each other and showed homology to human TCL1b. Additionally, a bacterial artificial chromosome (BAC) library was screened and three clones containing murine Tcl1 were obtained. PCR analysis of these BAC clones confirmed the presence of all three EST sequences. By a combination of RACE and RT-PCR experiments, database analysis of EST sequences and sequencing of selected EST clones, full length cDNAs corresponding to these sequences were isolated.

Because of the shared similarity among the cDNAs it was not possible to obtain unique probes for each. Thus, the genomic structure of the region by conventional methods such as Southern hybridization and pulse field gel analysis could not be determined. Subsequently, the BAC (452-124) was sequenced and the position and the exon-intron boundaries of the three cDNAs was determined.

Further analysis of the region also revealed that it contains two other sequence related genes. RT-PCR experiments with specific primers for these two genes confirmed that they are transcribed. Altogether, five full length cDNAs (SEQ. ID. NO: 47-51) were isolated located on murine chromosome 12 centromeric to the Igh locus homologous to human TCL1b. Murine Tcl1b1-Tcl1b5 cDNAs had a length of ˜1 kb (SEQ. ID. NO: 47-51, respectively) encoding for proteins ranging in size from 117-123 amino acids (SEQ. ID. NO: 57-63, respectively). They share 70-90% nucleic acid homology and 55-75% amino acid identity and 65-80% amino acid similarity. The murine Tcl1b family members show ˜25% identity and ˜35% similarity to murine Tcl1 and are 25-30% identical and 30-40% similar to human TCL1b.

The five genes are aligned on murine chromosome 12 (FIG. 5C) in tire order Tcl1b2, Tcl1b1, Tcl1b5, Tcl1b3, and Tcl1b4 with distances of 4.5 kb, 9.7 kb, 9.9 kb, and 6.8 kb, respectively, from each other and 9.8 kb between Tcl1b4 and Tcl1. The total sizes of the genes are: Tcl1b1: 6.9 kb, (SEQ. ID. NO: 52); Tcl1b2: 8.2 kb, (SEQ. ID. NO: 53); Tcl1b3 (SEQ. ID. NO: 54); and Tcl1b4: 4.6 kb, (SEQ. ID. NO: 55); Tcl1b5: 4.8 kb (SEQ. ID. NO: 56). The direction of transcription of Tcl1b1-Tcl1b5 is opposite to that of Tcl1. Each of the murine Tcl1b genes contains four exons of approximately 200, 170, 70, and 590 bp in size. The only exceptions are the exons 3 of Tcl1b2 and Tcl1b4, in which a different splicing site leads to a transcript 29 bp shorter. In addition, sequences of RT-PCR and RACE products and ESTs derived from Genebank showed alternatively spliced cDNAs for Tcl1b1 and Tcl1b3. Tcl1b1 may have a deletion of 73 bp consisting of nearly the complete exon 3 and the first 6 bp of exon 4. Because this deletion includes the stop codon the deduced protein sequence is slightly longer (Tcl1b1a, SEQ. ID. NO: 58). For Tcl1b3 an alternative exon 1 was found leading to a shorter protein (Tcl1b3a, SEQ. ID. NO: 61) with an alternative N-terminal end without homology to other Tcl1b proteins.

Although the homology of murine Tcl1b proteins (SEQ. ID. NO: 57-63) to human Tcl-1b (SEQ. ID. NO: 39) is lower than typically observed between mouse and human homologues (70-100%), the position of the genes on the map, their direction of transcription and their exon-intron structure are similar to the human TCL1b locus and indicate that these genes are authentic homologues to the human TCL1b gene (SEQ. ID. NO: 40).

Expression of Human TNG1 and TNG2.

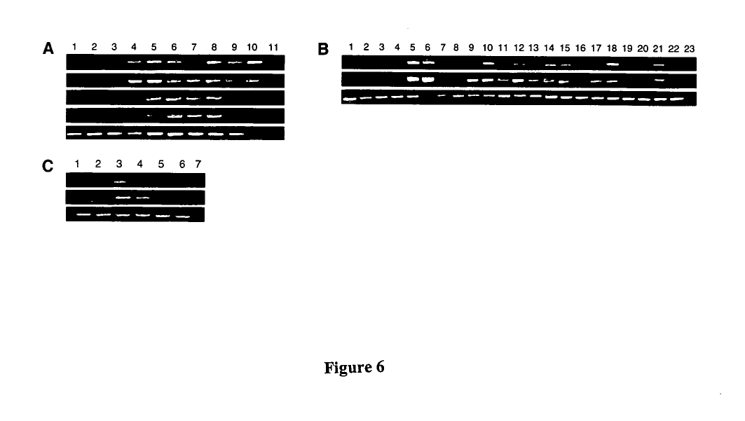

??Because TNG1 and TNG2 are located at the same locus as TCL1 and TCL1b, it seemed possible that they would exhibit similar expression patterns. To investigate this, a series of Northern blot and RT-PCR experiments were performed. TNG1 and TNG2 are both transcribed in a wide variety of normal tissues (FIG. 6B). The results demonstrate a low level of expression in most tissues examined including placenta, kidney, fetal kidney, fetal lung, and fetal heart and all lymphoid tissues including fetal liver and fetal spleen. The only exception is thymus, which only showed transcripts of TNG2, whereas fetal thymus only expressed TNG1 (FIG. 6B). TCL1b was expressed in the same tissues as TNG1 except thymus, fetal lung, and fetal heart ???(Virgilio, L., et al., 1998, Proc Natl Acad Sci USA, 95:3885-3889). Northern blot analysis of normal adult and embryonic tissues was negative for TNG1 and TNG2, probably due to the low level of expression.

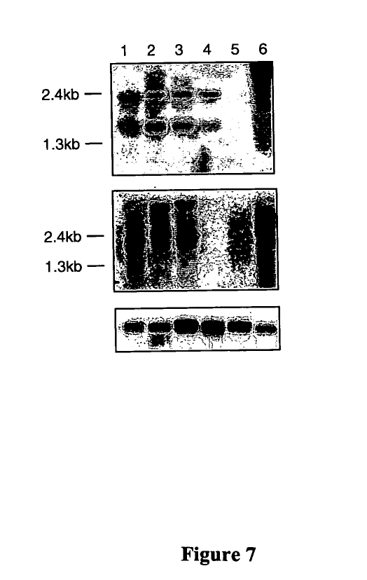

Because of the similarity of transcription patterns of TNG1 and TNG2 to those of TCL1 and especially TCL1b, and the physical linkage of these genes, the activation of the TNG genes by rearrangements at 14q32.1 was investigated. FIG. 6A demonstrates that all four genes show an identical expression pattern in lymphoid tumor cell lines. They are all expressed in early B-tumor cell lines (697, Raji, Daudi, and CA-46), but not in postthymic T-cell lines without 14q32.1 rearrangements. Nevertheless, TCL1, TCL1b, TNG1, and TNG2 are all transcribed in the T-ALL cell line SupT11 carrying a t(14;14)(q11;q32) translocation. Northern blot experiments confirmed these transcription patterns (FIG. 7). The 1.5 kb transcript of TNG1 was found in Burkitt's lymphoma cell lines Daudi and CA-46 and to a lesser extent also in the Raji cell line and in the T-cell acute lymphocytic leukemia cell line SupT11 (T-ALL) that carries a 14q32.1 translocation. The second band, a ˜2.3 kb transcript is likely to be a product of alternative splicing or incompletly processed hnRNA. However, activation of TNG2 in the SupT11 cell line was not confirmed by Northern blotting, due to a lower expression level. The 2 kb TNG2 transcript was detected in all three Burkitt's lymphoma cell lines, but not in the pre B-cell line 697 or in any of the T-cell lines investigated. The diffuse signal around the bands is due to the various alternative splicing products known to involve these gene.

To further study the activation of TNG1 and TNG2 by rearrangements at 14q32.1 the expression of TNG1 and TNG2 was investigated in four T-cell prolymphocytic leukemias (T-PLL) overexpressing TCL1. FIG. 6C shows the activation of both genes in 2 out of 4 cases. The transcripts of TNG1 and TNG2 were detected after 27 cycles of PCR in these two cases even though at these conditions bone marrow and peripheral blood lymphocytes were negative. Interestingly, activation of TCL1b in one of the two cases not expressing the TNG genes ???(Pekarsky, Y., et al., 1999, Proc Nail Acad Sci USA, 96:2949-2951) was previously found. These results indicate that juxtaposition of the TCL1 locus at 14q32.1 to the α/δ locus of the T-cell receptor activates TNG1 and TNG2, as well as TCL1 and TCL1b.

Expression of Murine Tcl1b Genes.

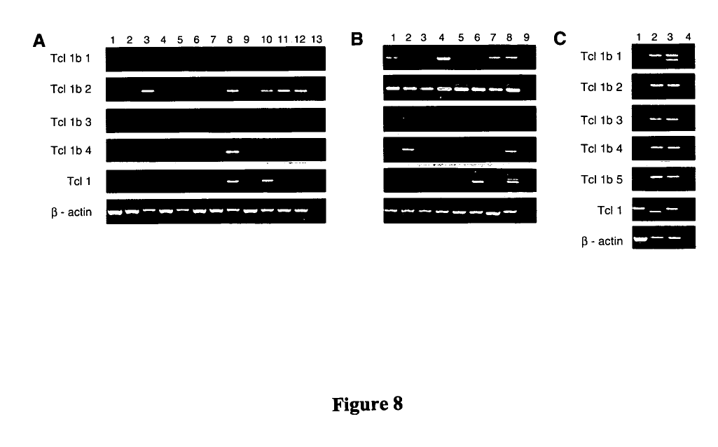

To investigate the expression pattern of the murine Tcl1b genes a series of RT-PCR experiments was carried out for each of the five genes. After a single round of PCR no mRNA expression was found in a series of normal tissue, embryonic cDNA libraries, and lymphoid cell lines for any of the five genes. However, nested PCR analysis revealed a low level of expression of Tcl1b2 in all lymphoid cell lines (FIG. 8B) and nearly all normal tissues Further, Tcl1b2 expression increased during embryonic developement (7-17 days old embryos, FIG. 8A). A low level of expression was also found for Tcl1b1 in nearly all lymphoid cell lines, but not in any other tissues. Tcl1b4 only showed a low level of expression in testis and in the pro B-cell line NFS 5, which also expressed Tcl1b3, as confirmed by sequencing (FIG. 8). Tcl1b5 expression was not detected in any tisuue or cell line examined. In comparison to the Tcl1b genes, Tcl1 was expressed at low levels in testis, 11 and 15 days old embryos and in the thymocyte cell line S49-1. Interestingly, murine Tcl1 was not detected in any of the early B-cell lines, although early B-cells show expression of TCL1 in humans (Virgilio, L., et al., 1994, Proc Natl Acad Sci USA, 95:3885-3889; Virgilio, L., et al., 1998, Proc Natl Acad Sci USA, 95:3885-3889).

Since the original three sets of ESTs all derived from a 2 cell embryonic cDNA library where they make up ˜0.5% of the total ESTs, cDNA from mouse embryonic stem (ES) cells, oocytes, and 2 cell embryos was investigated for the expression of Tcl1b genes. After a single round of RT-PCR, expression of all five Tcl1b genes and Tcl1 was found in mouse oocytes and 2 cell embryos at a level comparable to that of β-actin expression (FIG. 8C). In 2 cell embryos, both splicing variants of Tcl1b1 were amplified. Interestingly, in the mouse oocyte cDNA library only a shorter transcript of Tcl1 was detected, missing a part of exon 2. Only Tcl1 showed expression in ES cells after a single round of PCR, but nested PCR revealed a low level of expression also of Tcl1b1, Tcl1b2, and Tcl1b4. The high expression of all five Tcl1b genes and Tcl1 in mouse oocytes and 2 cell embryos implies that an important function of these genes occurs in the early embryogenesis of the mouse.

Protein Structure of TCL1 Family.

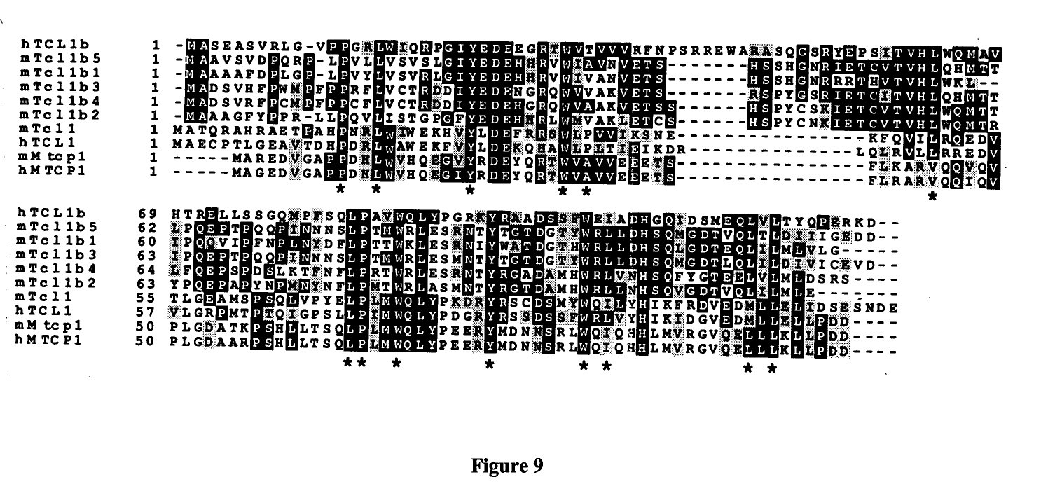

Tcl1 and Mtcp1 proteins both consist of an eight-stranded antiparallel β-barrel with a hydrophobic core and are predicted to bind small hydrophobic ligands (Fu, Z. Q., et al., 1998, Proc Natl Acad Sci USA, 95:3413-3418). Amino acid sequence alignment of these proteins with human and mouse Tcl1b (FIG. 9) shows that, despite only an overall 30-40% homology, all 14 amino acids forming the hydrophobic core are conserved except Pro36. 10 of these 14 amino acids are identical in all 10 members of the Tcl1 family, whereas three residues show conservative substitutions in some of the proteins (Leu49->Val, Leu92->Ile, Met104->Leu). Therefore, those residues have an important function in all Tcl1 family members.

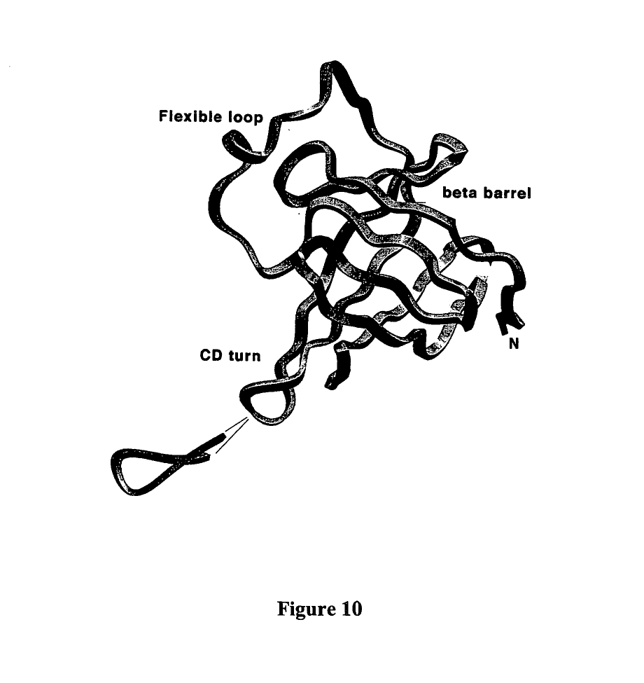

Human Tcl1b (SEQ. ID. NO: 39) shows a 14 residue insertion (Arg44-Glu58) relative to human TCl1 (FIG. 9). Mouse Tcl1b has a smaller, 10-11 residue insertion in the same position. A molecular model was built for human and murine Tcl1b based on the 35% similarity in amino acid sequence to Tcl1. In this model, the Tcl1b insertion aligns with a non-canonical, 5 residue turn (Lys42-Gln46) observed in the crystal structure of human Tcl1 (Hoh, F., et al., 1998, Structure, 6:147-155). The additional residues in human and mouse Tcl1b may form a surface accessible beta-sheet extension or a flexible loop with conserved charged amino acids (FIG. 10).

Discussion

The present invention discloses the cloning, mapping and expression analysis to of a novel member of the TCL1 gene family, TCL1b. The TCL1 and TCL1b genes are physically linked, show structural similarity, similar expression patterns and involvement in T-cell malignancies. Because the remaining two members of the TCL1 family are oncogenes (Virgilio, L., et al., 1998, Proc Natl Acad Sci USA, 95:3885-3889; Gritti, C., et al., 1998, Blood, 92:368-373), it seems likely that TCL1b is also an oncogene. It is also likely that TCL1b activation would explain cases of T-cell leukemia with amplification at 14q32 without activation of TCL1.

It is possible that two TCL1 genes are the result of duplication, although the TCL1b gene is slightly more homologous to the MTCP1 gene at Xq28 than to the TCL1 gene.

Neither the in vivo function of Tcl1, nor the mechanism(s) of its oncogenic potential is known, although its crystal structure (Fu, Z. Q., et al., 1998, Proc Natl Acad Sci USA, 95:3413-3418) suggests, it may function as a transporter of small molecules, such as retinoids, nucleosides or fatty acids. The same study (Fu, Z. Q., et al., 1998, Proc Natl Acad Sci USA, 95:3413-3418). suggested that Tcl1 might function as dimer, implying the possibility that Tcl1 and Tcl1b might form heterodimers.

Since TCL1 and MTCP1 transgenic mice develop mature T-cell leukemia only after 15 months (Virgilio, L., et al., 1998, Proc Natl Acad Sci USA, 95:3885-3889; Gritti, C., et al., 1998, Blood, 92:368-373), it will be of considerable interest to determine whether TCL1b transgenic mice also develop mature T-cell leukemia late and whether TCL1 and TCL1b double transgenic mice develop leukemia faster. Thus, is seems possible that translocations and inversions at 14q32.1 contribute to malignant transformation by activating two oncogenes at the same time.

The present invention discloses the cloning, mapping, and expression analysis of the human and murine TCL1/Tcl1 locus. Human TCL1 and TCL1b genes (SEQ. ID. NO: 40); are located between two clusters of chromosomal breakpoints and are activated by translocations and inversions at 14q32.1 juxtaposing them to regulatory elements of T-cell receptor genes (Pekarsky, Y., et al., 1999, Proc Natl Acad Sci USA, 96:2949-2951). Between these two sets of breakpoints two new genes were found and characterized, TNG1 and TNG2 (SEQ. ID. NO: 45 and 46, respectively). Both show no homology to any known genes, but similar expression patterns to that of TCL1 and TCL1b. Both TNG genes are also activated in the T-cell leukemia cell line SupT11 carrying a t(14;14) translocation, and in two out of four T-PLL samples. Therefore, like TCL1 and TCL1b, these two genes are also activated by rearrangements at 14q32.1 involved in T-cell malignancies. Thus, T-cell leukemias, in some cases, are induced by the activation of a single gene or in others by the cumulative activation of two or more of these four genes, although the oncogenic potential of TNG1 and TNG2 remains to be determined.

To assist in the structural and functional analysis of the human TCL1 gene activation, the murine Tcl1 locus was searched for homologues to human TCL1b and TNG genes. Five genes homologous to human TCL1b were found. The high shared similarity between the five murine Tcl1b genes (SEQ. ID. NO: 52-56), not only in the exons but also in intronic sequences, implies that they are most likely a result of duplications. All five genes are transcribed into mRNA but it remains to be determined whether they all code for active proteins or whether some of them might be pseudogenes. Their genomic structure, though, is untypical for pseudogenes, since it includes introns.

The five Tcl1b genes show different expression patterns, suggesting different regulatory elements for each of them, but since the expression of all of the genes in all adult tissues and cell lines is very low, the significance of this is not clear. Interestingly, the expression of murine Tcl1 and Tcl1b genes in lymphoid tissues and cell lines is much lower than the expression of their human homologues. The most striking feature of murine Tcl1 and Tcl1b genes is their very high expression level (up to 0.5% of all mRNA) in mouse oocytes and 2-cell embryos. This finding is consistent with the presence of human TCL1b in a syncytiotrophoblast subtracted cDNA library (genebank accession # AF137027), implying a function of murine and human TCL1b genes in the early embryogenesis.

The identification of five more murine members of the Tcl1 family provides a better understanding of the structural differences and similarities between the Tcl1 family of proteins. A comparison of the protein sequences of all members of the family including murine and human MTCP1 shows that, although overall homologies between the genes are low, the hydrophobic core region as described by Fu et al. (14) is preserved. This indicates a similar function for all of these proteins as transporters of small molecules such as retinoids, nucleosides, or fatty acids as suggested previously for Tcl1 and Mtcp1 (Hoh, F., et al., 1998, Structure, 6:147-155; Fu, Z. Q., et al., 1998, Proc Natl Acad Sci USA, 95:3413-3418). However, compared to Tcl1 and Mtcp1 mouse and human Tcl1b proteins show an insertion which form a surface accessible flexible loop or beta-sheet extension. The conserved charged residues in the insert loop play a significant role in mediating interactions with other proteins or ligands and also influence the quaternary structure of mouse Tcl1b (Hoh, F., et al., 1998, Structure, 6:147-155).

Altogether, murine and human TCL1 loci show significant differences: There are five murine Tcl1b genes compared to one human TCL1b. The homology of human and mouse TCL1b is low and the expression levels of murine Tcl1 and Tcl1b in lymphoid tissues and cell lines is much lower than the expression levels of their human equivalents. Moreover, murine homologues of TNG1 and TNG2 were not found. This implies that there are also be significant differences in the function of human and mouse TCL1 loci. Further investigation should lead to a better understanding of the role of Tcl1 and Tcl1b in normal development and T cell leukemia.

The present invention relates to nucleotide sequences of TCL-1b (SEQ. ID. NO: 40); TNG1 (SEQ. ID. NO: 45); and TNG2 (SEQ. ID. NO: 46); genes and amino acid sequences of their encoded Tcl-1b (SEQ. ID. NO: 39);, TNG1 (SEQ. ID. NO: 42); and TNG2 (SEQ. ID. NO: 44), respectively, proteins, as well as derivatives and analogs thereof, and antibodies thereto. The present invention further relates to the use of TCL-1b, TNG1 and TNG2 genes and their encoded proteins or derivatives or analogs thereof, and antibodies thereto, in assays for the detection and in treatment/prevention of disease states associated with chromosomal abnormalities and/or increased expression of TCL1b, TNG1 and TNG2. The present invention also relates to therapeutic compositions comprising Tcl-1b, TNG1 and TNG2, proteins, derivatives or analogs thereof, antibodies thereto, nucleic acids encoding these proteins, derivatives or analogs, and antisense nucleic acids.

The TCL-1b, TNG1 and TNG2 gene sequences are from one of many different species, including but not limited to, mammalian, bovine, ovine, porcine, equine, rodent and human, in naturally occurring sequence or in variant form, or from any source, whether natural, synthetic, or recombinant. In a specific embodiment described herein, the TCL-1b, TNG1 and TNG2 gene sequence are human sequences. The Tcl-1b, Tng1 and Tng1 proteins are those present in one of many different species, including but not limited to, mammalian, bovine, ovine, porcine, equine, rodent and human, in naturally occurring or variant form, or from any source, whether natural, synthetic, or recombinant. In the specific embodiment described herein, the above proteins are human proteins.

As defined herein, a Tcl-1b, Tng1 and Tng2 derivative is a fragment or amino acid variant of the Tcl-1b, Tng1 and Tng2 sequence (SEQ. ID. NO: 39, 42, and 44), respectively, as long as the fragment or amino acid variant is capable of displaying one or more biological activities associated with the full-length proteins. Such biological activities include, but are not limited to, antigenicity, i.e., the ability to bind to an their respective antibodies, and immunogenicity, i.e., the ability to generate an antibody which is capable able of binding a Tcl-1b, Tng1 or Tng2 protein, respectively.

The invention provides fragments of a Tcl-1b, Tng1 or Tng2 protein consisting of at least 10 amino acids, or of at least 25 amino acids, or of at least 50 amino acids, or of at least 114 amino acids. Nucleic acids encoding such derivatives or analogs are also within the scope of the invention. A preferred Tcl-1b, Tng1 or Tng2 protein variant is one sharing at least 70% amino acid sequence homology, a particularly preferred Tcl-1b, Tng1 or Tng2 protein variant is one sharing at least 80% amino acid sequence homology and another particularly preferred Tcl-1b, Tng1 or Tng2 protein variant is one sharing at least 90% amino acid sequence homology to the naturally occurring Tcl-b, Tng1 or Tng2 protein over at least 25, at least 50, at least 75 or at least 100 contiguous amino acids of the Tcl-1b, Tng1 or Tng2 amino acid sequence, respectively. As used herein, amino acid sequence homology refers to amino acid sequences having identical amino acid residues or amino acid sequences containing conservative changes in amino acid residues. In another embodiment, a Tcl-1b, Tng1 or Tng2 homologous protein is one that shares the foregoing percentages of sequences identical with the naturally occurring Tcl-1b, Tng1 or Tng2 protein, respectively, over the cited lengths of amino acids.

The TCL-b1, TNG1 and TNG2 genes (SEQ. ID. NO: 40, 45, and 46, respectively), are located in the region of chromosome 14q32.1 that is located in a region banded by two clusters of breakpoints. Due to the similarities between the TCL1 and TCL-1b gene structure, sequence and location, their expression patterns were compared. In addition the expression patterns of TNG1 and TNG2, which are located at the same locus as that of TCL1 and TCL-1b, were investigated (FIG. 7). Expression in normal tissue was mostly negative for TCL1b, FIG. 7A. The TCL1 gene expression, however, was detected in most hematopoietic tissues and both TCL1 and TCL1b are expressed in spleen, tonsil, fetal liver, fetal kidney and fetal thymus. The TCL1b gene (SEQ. ID. NO: 39) is expressed in a wider variety of tissues including placenta, kidney and fetal spleen, as shown in FIG. 8A. Low levels of expression of TNG1 and TNG2 were present in most tissues examined, in addition to those for TCL1b, expression in fetal lung, fetal heart and fetal liver. The only exception is the thymus, which showed transcripts of TNG2, whereas fetal thymus only expressed TNG1 (FIG. 6B). The detection of TCL-1b, TNG1 and TNG2 mRNA in patient samples, such as biopsied cells and tissues, is used as an indicator of the presence of T-cell leukemias and lymphomas associated with certain chromosome 14 abnormalities and/or increased expression of Tcl-1b, Tng1 or Tng2 proteins. Also, the Tcl-1b, Tng1 or Tng2 amino acid sequences of the present invention (SEQ. ID. NO: 39, 42, and 44, respectively), are used to generate antibodies useful in immunoassays for the detection or measurement of Tcl-1b, Tng1 or Tng2 proteins in patient samples, respectively. Such antibodies are used in diagnostic immunoassays, for the detection or measurement of increased levels of Tcl-1b, Tng1 or Tng2 proteins, respectively, associated with T-cell leukemias and lymphomas.

In accordance with the present invention, polynucleotide sequences coding for a Tcl-1b, Tng1 or Tng2 proteins (SEQ. ID. NO: 38, 41, and 43), derivatives, e.g. fragment, or analog thereof, are inserted into an appropriate expression vector, i.e., a vector which contains the necessary elements for the transcription and translation of the inserted protein-coding sequence, for the generation of recombinant DNA molecules that direct the expression of a Tcl-1b, Tng1 or Tng2 proteins. Such Tcl-1b, Tng1 or Tng2 polynucleotide sequences, as well as other polynucleotides or their complements, are also used in nucleic acid hybridization assays, Southern and Northern blot analysis, etc. In a specific embodiment, a human TCL-1b, TNG1 or TNG2 gene (SEQ. ID. NO: 40, 45, and 46, respectively), or a sequence encoding a functionally active portion of a human TCL-1b, TNG1 or TNG2 gene, is expressed. In yet another embodiment, a derivative or fragment of a human TCL-1b, TNG1 or TNG2 gene is expressed.

The TCL-1b, TNG1 and TNG2 Coding Sequences

In a specific embodiment disclosed herein the invention relates to the nucleic acid sequence of the human TCL-1b, TNG1 and TNG2 genes (SEQ. ID. NO: 40, 45, and 46, respectively). In a preferred, but not limiting, aspect of the invention, a human TCL-1b cDNA sequence (SEQ. ID. NO: 38) was identified in the expressed sequence tag database (accession no. AA689513) that was homologous to TCL-1 and MTCP1, the other members of the TCL1 gene family. Such a sequence was isolated and cloned as a 1.2 kilobase full-length cDNA, as described, supra. The TNG1 and TNG2 genes were also isolated and identified and their sequences compared to the expressed sequence database (EST). The 1.5 kilobase cDNA of TNG1 (SEQ. ID. NO: 41) contains an open reading frame encoding a protein of 141 amino acids and the 2 kilobase TNG2 gene (SEQ. ID. NO: 43) encodes a protein of 110 amino acids, as described, supra. The invention also relates to nucleic acid sequences hybridizable or complementary to the foregoing sequences, of equivalent to the foregoing sequences, in that the equivalent nucleic acid sequences also encode a Tcl-1b, Tng1 or Tng2 protein product.

In a preferred aspect, polymerase chain reaction (PCR) is used to amplify the desired nucleic acid sequence in the library by using oligonucleotide primers representing known TCL-1b, TNG1 or TNG2 sequences (SEQ. ID. NO: 38, 41, and 43, respectively). Such primers are used to amplify sequences of interest from an RNA or DNA source, preferably a cDNA library. PCR is carried out by use of a Perkin-Elmer Cetus thermal cycler and Taq polymerase, as is well known by those skilled in the art. The DNA being amplified is mRNA or cDNA or genomic DNA from any eukaryotic species. Several different degenerate primers are synthesized for use in PCR amplification reactions. The stringency of hybridization conditions used in priming the PCR reactions are also varied in order to allow for greater or lesser degrees of nucleotide sequence homology between the TCL-1b, TNG1 or TNG2 gene being cloned and that of the TCL-1b, TNG1 or TNG2 genes (SEQ. ID. NO: 40, 45, and 46, respectively) of the present invention.

After successful amplification of a segment of the TCL-1b, TNG1 or TNG2 gene, an allelic, a polymorphic variant, or a species homology of the TCL-1b, TNG1 or TNG2 gene, that segment is molecularly cloned and sequenced, and utilized as a probe to isolate a complete cDNA or genomic clone. This will permit the determination of the gene's complete nucleotide sequence, the analysis of its expression, and the production of its protein product for functional analysis. This allows for the identification of additional genes encoding the Tcl-1b, Tng1 or Tng2, respectively, proteins.

Potentially, any eukaryotic cell can serve as the nucleic acid source for the molecular cloning of the TCL-1b, TNG1 or TNG2 gene. The nucleic acid sequences encoding TCL-1b, TNG1 or TNG2 gene are isolated from, for example, human, porcine, bovine, feline, avian, equine, canine, rodent, as well as additional primate sources. The DNA is obtained by standard procedures known in the art from, for example, cloned DNA (e.g., a DNA “library”), by chemical synthesis, by cDNA cloning, or by the cloning of genomic DNA, or fragments thereof, purified from a desired cell. (See, for example, Sambrook et al., 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. I, II.) A preferred source is cDNA of leukemic cells in which the leukemia is associated with a 14q32.1 chromosomal abnormality. Clones derived from genomic DNA contain regulatory and intron DNA regions in addition to coding regions, while clones derived from cDNA will contain only TCL-1b exon sequences. In a particular embodiment of the present invention, a genomic sequence is one that is not more than 10 kilobases (kb), or not more than 20 kb, or not more than 50 kb or not more than 70 kb. Whatever the source, the gene should be molecularly cloned into a suitable vector for propagation of the gene. In a particular embodiment, a preferred source of nucleic acid for the isolation of TCL-1b, TNG1 or TNG2 gene sequences is from pre B-cells.

In the molecular cloning of the gene from genomic DNA, DNA fragments are generated, some of which will encode the desired gene. The DNA is cleaved at specific sites using various restriction enzymes. Alternatively, DNAse in the presence of manganese is used to fragment the DNA, or the DNA is physically sheared, as for example, by sonication. The linear DNA fragments is then separated according to size by standard techniques, including but not limited to, agarose and polyacrylamide gel electrophoresis and column chromatography.

Once the DNA fragments are generated, identification of the specific DNA fragment containing the desired gene is accomplished in a number of ways. For example, a TCL-1b, TNG1 or TNG2 gene (SEQ. ID. NO: 40, 45, and 46, respectively) of the present invention or its specific RNA, or a fragment thereof, such as a probe or primer, is isolated and labeled and then used in hybridization assays to detect a generated TCL-1, TNG1 or TNG2 gene (Benton, W. and Davis, R., 1977, Science, 196:180; Grunstein, M. And Hogness, D., 1975, Proc Natl Acad Sci USA, 72:3961). Those DNA fragments sharing substantial sequence homology to the probe will hybridize under stringent conditions. The phrase “stringent conditions” as used herein refers to those hybridizing conditions that (Virgilio, L., et al., 1994, Proc Natl Acad Sci USA, 91:12530-12534) employ low ionic strength and high temperature for washing, for example, 0.015 M NaCl/0.0015 M sodium citrate/0.1% SDS at 50.degree. C.; (Narducci, M. G., et al., 1997, Cancer Res, 57:5452-5456) employ, during hybridization, a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42.degree. C.; or (Virgilio, L., et al., 1998, Proc Natl Acad Sci USA, 95:3885-3889) employ 50% formamide, 5.times.SSC (0.75 M NaCl, 0.075 M sodium pyrophosphate, 5.times.Denhardt's solution, sonicated salmon sperm DNA (50 g/ml), 0.1% SDS, and 10% dextran sulfate at 42.degree. C., with washes at 42.degree. C. in 0.2.times.SSC and 0.1% SDS.

The appropriate fragment is also identified by restriction enzyme digestion(s) and comparison of fragment sizes with those expected according to a known restriction map. Further selection is carried out on the basis of the properties of the gene. Alternatively, the presence of the gene is detected by assays based on the physical, chemical, or immunological properties of its expressed product. For example, cDNA clones, or genomic DNA clones which hybrid-select the proper mRNAs, are selected which produce a protein that has similar or identical electrophoretic migration, isolectric focusing behavior, proteolytic digestion maps, binding activity or antigenic properties as known for Tcl-1b. Alternatively, the Tcl-1b protein may be identified by binding of labeled antibody to the putatively Tcl-1b expressing clones, e.g., in an ELISA (enzyme-linked immunosorbent assay)-type procedure.

The TCL-1b, TNG1 or TNG2 gene is also identified by mRNA selection by nucleic acid hybridization followed by in vitro translation. In this procedure, fragments are used to isolate complementary mRNAs by hybridization. Such DNA fragments may represent available, purified TCL-1b, TNG1 or TNG2 DNA of another TCL-1b, TNG1 or TNG2 gene, respectively. Immunoprecipitation analysis, or functional assays, of the in vitro translation products of the isolated products of the isolated mRNAs identifies the mRNA and, therefore, the complementary DNA fragments that contain the desired sequences. In addition, specific mRNAs are selected by adsorption of polysomes isolated from cells to immobilized antibodies specifically directed against Tcl-1b, Tng1 or Tng2 protein. A radiolabelled TCL-1b, TNG1 or TNG2 cDNA is synthesized using the selected mRNA (from the adsorbed polysomes) as a template. The radiolabeled mRNA or cDNA is then used as a probe to identify the TCL-1b, TNG1 or TNG2 DNA fragments, respectively, from among other genomic DNA fragments.

Alternatives to isolating the TCL-1b, TNG1 or TNG2 genomic DNA include, but are not limited to, chemically synthesizing the gene sequence itself from a known sequence or making cDNA to the mRNA which encodes the Tcl-1b, Tng1 or Tng2, respectively, protein. For example, RNA useful in cDNA cloning of the TCL-1b, TNG1 or TNG2 gene is isolated from cells which express Tcl-1b, Tng1 or Tng2, respectively, e.g., pre-B acute lymphoblastic leukemia cells or endemic Burkitt's lymphoma cells which express cell surface IgM and do not secrete immunoglobulin. Other methods are known to those of skill in the art and are within the scope of the invention.

The identified and isolated gene is then inserted into an appropriate cloning vector. A large number of vector-host systems known in the art may be used. Possible vectors include, but are not limited to, plasmids or modified viruses, but the vector system must be compatible with the host cell used. Such vectors include, but are not limited to, bacteriophages such as lambda derivatives, or plasmids such as PBR322 or pUC plasmid derivatives. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers comprise specific chemically synthesized oligonucleotides encoding restriction endonuclease recognition sequences. In an alternative method, the cleaved vector and TCL-1b, TNG1 or TNG2 gene is modified by homopolymeric tailing. Recombinant molecules are introduced into host cells via transformation, transfection, infection, electroporation, or other methods known to those of skill in the art, so that many copies of the gene sequence are generated.

In an alternative method, the desired gene is identified and isolated after insertion into a suitable cloning vector in a “shot gun” approach. Enrichment for the desired gene, for example, by size fractionization, is done before insertion into the cloning vector.

In specific embodiments, transformation of host cells with recombinant DNA molecules that incorporate the isolated TCL-1b, TNG1 or TNG2 gene, cDNA, or synthesized DNA sequence enables generation of multiple copies of the gene. Thus, the gene is obtained in large quantities by growing transformants, isolating the recombinant DNA molecules from the transformants and, when necessary, retrieving the inserted gene from the isolated recombinant DNA.

Oligonucleotides containing a portion of the TCL-1b, TNG1 or TNG2 coding or non-coding sequences, or which encode a portion of the Tcl-1b, Tng1 or Tng2, respectively, protein (e.g., primers for use in PCR) are synthesized by standard methods commonly known in the art. Such oligonucleotides preferably have a size in the range of 8 to 25 nucleotides. In a particular embodiment herein, such oligonucleotides have a size in the range of 15 to 25 nucleotides or 18 to 25 nucleotides.

Expression of the TCL-1b, TNG1 or TNG2 Gene

In accordance with the present invention, polynucleotide sequences coding for a Tcl-1b, Tng1 or Tng2 protein, derivative, e.g. fragment, or analog thereof, can be inserted into an appropriate expression vector, i.e., a vector which contains the necessary elements for the transcription and translation of the inserted protein-coding sequence, for the generation of recombinant DNA molecules that direct the expression of a Tcl-1b, Tng1 or Tng2 protein. Such TCL-1b, TNG1 or TNG2, respectively, polynucleotide sequences, as well as other polynucleotides or their complements, may also be used in nucleic acid hybridization assays, Southern and Northern blot analysis, etc. In a specific embodiment, a human TCL-1b, TNG1 or TNG2 gene, or a sequence encoding a functionally active portion of a human TCL-1b, TNG1 or TNG2 gene is expressed. In yet another embodiment, a derivative or fragment of a human TCL-1b, TNG1 or TNG2 gene is expressed.

Due to the inherent degeneracy of the genetic code, other DNA sequences which encode substantially the same or a functionally equivalent Tcl-1b amino acid sequence, is within the scope of the invention. Such DNA sequences include those which are capable of hybridizing to the human TCL-1b, TNG1 or TNG2 sequence under stringent conditions.

Altered DNA sequences which are used in accordance with the invention include deletions, additions or substitutions of different nucleotide residues resulting in a sequence that encodes the same or a functionally equivalent gene product. The gene product itself may contain deletions, additions or substitutions of amino acid residues within a TCL-1b, TNG1 or TNG2 sequence, which result in a silent change, thus producing a functionally equivalent Tcl-1b, Tng1 or Tng2, respectively, protein. Such amino acid substitutions are made on the basis of similarity in polarity, charge, solubility, hydrophobicity, hydrophilicity, and/or the amphipathic nature of the residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, alanine; asparagine, glutamine; serine, threonine; phenylalanine, tyrosine.

The DNA sequences of the invention are engineered in order to alter a TCL-1b, TNG1 or TNG2 coding sequence for a variety of ends, including but not limited to alterations which modify processing and expression of the gene product. For example, mutations introduced using techniques which are well known in the art, e.g., site-directed mutagenesis, to insert new restriction sites, to alter phosphorylation, etc.

In another embodiment of the invention, a TCL-1b, TNG1 or TNG2 gene sequence or a derivative thereof is ligated to a non-TCL-1b, non-TNG1 or non-TNG2 gene sequence to encode a chimeric fusion protein. A fusion protein is engineered to contain a cleavage site located between a Tcl-1b, Tng1 or Tng2, respectively, sequence and the non-Tcl-1b, non-Tng1 or non-Tng2, respectively, protein sequence, so that the Tcl-1b, Tng1 or Tng2 protein, respectively may be cleaved away from the non-Tcl-1b, non-Tng1 or non-Tng2, respectively, moiety. In a specific embodiment, the Tcl-1b, non-Tng1 or non-Tng2, respectively, amino acid sequence present in the fusion protein consists of at least 10 contiguous amino acids, at least 25 contiguous amino acids, at least 50 contiguous amino acids, at least 75 contiguous amino acids, at least 100 contiguous amino acids, or at least 114 amino acids of the Tcl-1b, non-Tng1 or non-Tng2, protein sequence.

In an alternate embodiment of the invention, the coding sequence of a Tcl-1b, Tng1 or Tng2, is synthesized in whole or in part, using chemical methods well known in the art. See, for example, Caruthers et al., 1980, Nuc. Acids Res. Symp. Ser. 7:215-233; Crea and Horn, 1980, Nuc. Acids Res. 9(10):2331; Matteucci and Caruthers, 1980, Tetrahedron Letters 21:719; and Chow and Kempe, 1981, Nuc. Acids Res. 9(12):2807-2817. Alternatively, the protein itself is produced using chemical methods to synthesize a Tcl-1b, Tng1 or Tng2 amino acid sequence in whole or in part. For example, peptides are synthesized by solid phase techniques, cleaved from the resin, and purified by preparative high performance liquid chromatography. (e.g., see Creighton, 1983, Proteins Structures And Molecular Principles, W. H. Freeman and Co., N.Y. pp. 50-60). The composition of the synthetic peptides may be confirmed by amino acid analysis or sequencing (e.g., the Edman degradation procedure; see Creighton, 1983, Proteins, Structures and Molecular Principles, W. H. Freeman and Co., N.Y., pp. 34-49.

In order to express a biologically active Tcl-1b, Tng1 or Tng2 protein or derivative thereof, a polynucleotide sequence encoding a Tcl-1b, Tng1 or Tng2, resepectively, protein, or a derivative thereof, is inserted into an appropriate expression vector, i.e., a vector which contains the necessary elements for the transcription and translation of the inserted coding sequence. The TCL-1b, TNG1 or TNG2 gene products, as well as host cells or cell lines transfected or transformed with recombinant TCL-1b, TNG1 or TNG2, respecitvely, expression vectors, are used for a variety of purposes. These include, but are not limited to, generating antibodies (i.e., monoclonal or polyclonal) that immunospecifically bind a Tcl-1b protein. Anti-Tcl-1b, antig-Tng1 or anti-Tng2 antibodies are used in detecting or measuring levels of a Tcl-1b, Tng1 or Tng2, respectively, protein in patient samples.

Methods which are well known to those skilled in the art are used to construct expression vectors containing a TCL-1b, TNG1 or TNG2 coding sequence and appropriate transcriptional/translational control signals. These methods include in vitro recombinant DNA techniques, synthetic techniques and in vivo recombination/genetic recombination. See, for example, the techniques described in Sambrook et al., 1989, Molecular Cloning, A Laboratory Manual 2d ed., Cold Spring Harbor Laboratory, N.Y. and Ausubel et al., 1989, Current Protocols in Molecular Biology, Greene Publishing Associates and Wiley Interscience, N.Y.

A variety of host-expression vector systems are utilized to express a TCL-1b, TNG1 or TNG2 coding sequence. These include, but are not limited to, microorganisms such as bacteria transformed with recombinant bacteriophage DNA, plasmid DNA or cosmid DNA expression vectors containing a TCL-1b, TNG1 or TNG2 coding sequence; yeast transformed with recombinant yeast expression vectors containing a TCL-1b, TNG1 or TNG2 coding sequence; insect cell systems infected with recombinant virus expression vectors (e.g., baculovirus) containing an TCL-1b, TNG1 or TNG2 coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a TCL-1b, TNG1 or TNG2 coding sequence; or animal cell systems. The expression elements of these systems vary in their strength and specificities. Depending on the host/vector system utilized, any of a number of suitable transcription and translation elements, including constitutive and inducible promoters, are used in the expression vector. For example, when cloning in bacterial systems, inducible promoters such as pL of bacteriophage lambda, plac, ptrp, ptac (ptrp-lac hybrid promoter) and the like are used; when cloning in insect cell systems, promoters such as the baculovirus polyhedrin promoter are used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 355 RNA promoter of CaMV; the coat protein promoter of TMV) are used; when cloning in mammalian cell systems, promotersderived from the genome of mammalian cells (e.g., metallothionein promoter) or from mammalian viruses (e.g., the adenovirus late promoter; the vaccinia virus 7.5 K promoter) are used; when generating cell lines that contain multiple copies of a TCL-1b, TNG1 or TNG2 DNA, SV40-, BPV- and EBV-based vectors are used with an appropriate selectable marker.

In bacterial systems, a number of expression vectors are advantageously selected depending upon the use intended for the Tcl-1b, Tng1 or Tng2 protein expressed. For example, when large quantities of Tcl-1b, Tng1 or Tng2 protein are produced for the generation of antibodies, vectors which direct the expression of high levels of fusion protein products that are readily purified are desirable. Such vectors include, but are not limited to, the E. coli expression vector pUR278 (Ruther, et al., 1983, EMBO J, 2:1791), in which the TCL-1b, TNG1 or TNG2 coding sequence are ligated into the vector in frame with the lac Z coding region so that a hybrid AS-lac Z protein is produced; pIN vectors (Inouye & Inouye, 1985, Nucleic Acids Res, 13:3101-3109; Van Heeke & Schuster, 1989, J Biol Chem, 264:5503-5509); and the like. pGEX vectors are also used to express foreign polypeptides as fusion proteins with glutathione S-transferase (GST). In general, such fusion proteins are soluble and easily purified from lysed cells by adsorption to glutathione-agarose beads followed by elution in the presence of free glutathione. The PGEX vectors are designed to include thrombin or factor Xa protease cleavage sites so that the cloned polypeptide of interest is released from the GST moiety.

In yeast, a number of vectors containing constitutive or inducible promoters are used. For a review see, Current Protocols in Molecular Biology, Vol. 2, 1988, Ed. Ausubel et al., Greene Publish. Assoc. & Wiley Interscience, Ch. 13; Grant et al., 1987, Expression and Secretion Vectors for Yeast, in Methods in Enzymology, Ed. Wu & Grossman, 1987, Acad. Press, N.Y. 153:516-544; Glover, 1986, DNA Cloning. Vol. II, IRL Press, Wash., D.C., Ch. 3; and Bitter, 1987, Heterologous Gene Expression in Yeast, Methods in Enzymology, Eds. Berger & Kimmel, Acad. Press, N.Y. 152:673-684; and The Molecular Biology of the Yeast Saccharomyces, 1982, Eds. Strathern et al., Cold Spring Harbor Press, Vols. I and II.

In cases where plant expression vectors are used, the expression of a TCL-1b, TNG1 or TNG2 coding sequence is driven by any of a number of promoters. For example, viral promoters such as the 35S RNA and 19S RNA promoters of CaMV (Brisson, et al., 1984, Nature, 310:511-514), or the coat protein promoter of TMV (Takamatsu, et al., 1987, EMBO J, 6:307-311) are used; alternatively, plant promoters such as the small subunit of RUBISCO (Coruzzi, et al., 1984, EMBO J, 3:1671-1680; Broglie, et al., 1984, Science, 224:838-843); or heat shock promoters, e.g., soybean hsp17.5-E or hsp17.3-B (Gurley, et al., 1986, Mol Cell Biol, 6:559-565) are used. These constructs are introduced into plant cells using Ti plasmids, Ri plasmids, plant virus vectors, direct DNA transformation, microinjection, electroporation, etc. For reviews of such techniques see, for example, Weissbach & Weissbach, 1988, Methods for Plant Molecular Biology, Academic Press, N.Y., Section VIII, pp. 421-463; and Grierson & Corey, 1988, Plant Molecular Biology, 2d Ed., Blackie, London, Ch. 7-9.

An alternative expression system which could be used to express a TCL-1b, TNG1 or TNG2 gene is an insect ystem. In one such system, Autographa californica nuclear polyhedrosis virus (AcNPV) is used as a vector to express foreign genes. The virus grows in Spodoptera frugiperda cells. A TCL-1b, TNG1 or TNG2 coding sequence is cloned into non-essential regions (for example the polyhedrin gene) of the virus and placed under control of an AcNPV promoter (for example, the polyhedrin promoter). Successful insertion of a TCL-1b, TNG1 or TNG2 coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (e.g., see Smith, et al., 1983, J Virol, 46:584; Smith, U.S. Pat. No. 4,215,051).

In mammalian host cells, a number of viral based expression systems are utilized. In cases where an adenovirus is used as an expression vector, a TCL-1b, TNG1 or TNG2 coding sequence is ligated to an adenovirus transcription/translation control complex, e.g., the late promoter and tripartite leader sequence. This chimeric gene is then inserted in the adenovirus genome by in vitro or in vivo recombination. Insertion in a non-essential region of the viral genome (e.g., region E1 or E3) will result in a recombinant virus that is viable and capable for expressing a TCL-1b in infected hosts. (e.g., see Logan & Shenk, 1984, Proc. Natl. Acad. Sci. U.S.A. 81:3655-3659). Alternatively, the vaccinia 7.5 K promoter are used. (See, e.g., Mackett, et al., 1982, Proc Natl Acad Sci USA, 79:7415-7419; Mackett, et al., 1984, J Virol, 49:857-864; Panicali, et al., 1982, Proc Natl Acad Sci USA, 79:4927-4931).

Specific initiation signals may also be required for efficient translation of an inserted TCL-1b, TNG1 or TNG2 coding sequences. These signals include the ATG initiation codon and adjacent sequences. In cases where an entire TCL-1b, TNG1 or TNG2 gene, including its own initiation codon and adjacent sequences, is inserted into the appropriate expression vector, no additional translational control signals may be needed. However, in cases where only a portion of a TCL-1b, TNG1 or TNG2 coding sequence is inserted, lacking the 5′ end, exogenous translational control signals, including the ATG initiation codon, must be provided. Furthermore, the initiation codon must be in phase with the reading frame of a TCL-1b, TNG1 or TNG2 coding sequence to ensure translation of the entire insert. These exogenous translational control signals and initiation codons of are of a variety of origins, both natural and synthetic. The efficiency of expression are enhanced by the inclusion of appropriate transcription enhancer elements, transcription terminators, etc. (see Bittner, et al., 1987, Methods in Enzymol, 153:516-544).

In addition, a host cell strain is chosen which modulates the expression of the inserted sequences, or modifies and processes the gene product in the specific fashion desired. Such modifications (e.g., phosphorylation) and processing (e.g., cleavage) of protein products may be important for the function of the protein. Different host cells have characteristic and specific mechanisms for the post-translational processing and modification of proteins. Appropriate cells lines or host systems are chosen to ensure the correct modification and processing of the foreign pry expressed. To this end, eukaryotic host cells which possess the cellular machinery for proper processing of the primary transcript, and phosphorylation of the gene product are used. Such mammalian host cells include but are not limited to CHO, VERO, BHK, HeLa, COS, MDCK, 293, WI38, etc.

For long-term, high-yield production of recombinant proteins, stable expression is preferred. For example, cell lines which stably express a Tcl-1b, Tng1 or Tng2 protein are engineered. Rather than using expression vectors which contain viral origins of replication, host cells are transformed with TCL-1b, TNG1 or TNG2 DNA, respectively, controlled by appropriate expression control elements (e.g., promoter, enhancer, sequences, transcription terminators, polyadenylation sites, etc.), and a selectable marker. Following the introduction of foreign DNA, engineered cells are allowed to grow for 1-2 days in an enriched media, and are then switched to a selective media. The selectable marker in the recombinant plasmid confers resistance to the selection and allows cells to stably integrate the plasmid into their chromosomes and grow to form foci which in turn are cloned and expanded into cell lines. This method is advantageously used to engineer cell lines which express a Tcl-1b, Tng1 or Tng2, respectively, protein. The present invention provides a method for producing a recombinant Tcl-1b, Tng1 or Tng2 protein comprising culturing a host cell transformed with a recombinant expression vector encoding a Tcl-1b, Tng1 or Tng2, respectively, protein such that the Tcl-1b, Tng1 or Tng2 protein is expressed by the cell and recovering the expressed Tcl-1b, Tng1 or Tng2 protein.

A number of selection systems are used, including, but not limited to, the herpes simplex virus thymidine kinase (Wigler, et al., 1977, Cell, 11:223), hypoxanthine-guanine phosphoribosyltransferase (Szybalska & Szybalski, 1962, Proc Natl Acad Sci USA, 48:2026), and adenine phosphoribosyltransferase (Lowy, et al., 1980, Cell, 22:817) genes can be employed in tk-, hgprt- or aprt-cells, respectively. Also, antimetabolite resistance is used as the basis of selection for dhfr, which confers resistance to methotrexate (Wigler, et al., 1980, Natl Acad Sci USA, 77:3567; O'Hare, et al., 1981, Proc Natl Acad Sci USA, 78:1527); gpt, which confers resistance to mycophenolic acid (Mulligan & Berg, 1981, Proc Natl Acad Sci USA, 78:2072), neo, which confers resistance to the aminoglycoside G-418 (Colberre-Garapin, et al., 1981, J Mol Biol, 150:1); and hygro, which confers resistance to hygromycin (Santerre, et al., 1984, Gene, 30:147). Recently, additional selectable genes have been described, namely trpB, which allows cells to utilize indole in place of tryptophan; hisD, which allows cells to utilize histinol in place of histidine (Hartman & Mulligan, 1988, Proc Natl Aca. Sci USA, 85:8047); and ODC (ornithine decarboxylase) which confers resistance to the ornithine decarboxylase inhibitor, 2-(difluoromethyl)-DL-ornithine, DFMO (McConlogue, L., 1987, In: Current Communications in Molecular Biology, Cold Spring Harbor Laboratory, Ed.).

Identification of Transfectants or Transformants that Express Tcl-1b, Tng1 or Tng2

The host cells which contain the coding sequence and which express the biologically active gene product are identified by at least four general approaches; (a) DNA-DNA or DNA-RNA hybridization; (b) the presence or absence of “marker” gene functions; (c) assessing the level of transcription as measured by the expression of TCL-1b, TNG1 or TNG2 mRNA transcripts in the host cell; and (d) detection of the gene product as measured by immunoassay or by its biological activity.

In the first approach, the presence of the TCL-1b, TNG1 or TNG2 coding sequence inserted in the expression vector is detected by DNA-DNA or DNA-RNA hybridization using probes comprising nucleotide sequences that are homologous to the TCL-1b, TNG1 or TNG2 coding sequence, respectively, or portions or derivatives thereof.

In the second approach, the recombinant expression vector/host system is identified and selected based upon the presence or absence of certain “marker” gene functions (e.g., thymidine kinase activity, resistance to antibiotics, resistance to methotrexate, transformation phenotype, occlusion body formation in baculovirus, etc.). For example, if the human TCL-1b, TNG1 or TNG2 coding sequence is inserted within a marker gene sequence of the vector, recombinant cells containing the TCL-1b, TNG1 or TNG2 coding sequence are identified by the absence of the marker gene function. Alternatively, a marker gene is placed in tandem with a TCL-1b, TNG1 or TNG2 sequence under the control of the same or different promoter used to control the expression of the TCL-1b, TNG1 or TNG2 coding sequence. Expression of the marker in response to induction or selection indicates expression of the TCL-1b, TNG1 or TNG2 coding sequence.

In the third approach, transcriptional activity of a TCL-1b, TNG1 or TNG2 gene is assessed by hybridization assays. For example, RNA is isolated and analyzed by Northern blot using a probe having sequence homology to a TCL-1b, TNG1 or TNG, respectively, coding sequence or transcribed noncoding sequence or particular portions thereof. Alternatively, total nucleic acid of the host cell are extracted and quantitatively assayed for hybridization to such probes.

In the fourth approach, the levels of a Tcl-1b, Tng1 or Tng2 protein product is assessed immunologically, for example by Western blots, immunoassays such as radioimmuno-precipitation, enzyme-linked immunoassays and the like.

Purification of the Expressed Gene Product

Once a recombinant which expresses the TCL-1b, TNG1 or TNG2 gene sequence is identified, the gene product is analyzed. This is achieved by assays based on the physical or functional properties of the product, including radioactive labelling of the product followed by analysis by gel electrophoresis, immunoassay, or other detection methods known to those of skill in the art.

Once the Tcl-1b, Tng1 or Tng2 protein is identified, it is isolated and purified by standard methods including chromatography (e.g., ion exchange, affinity, and sizing column chromatography), centrifugation, differential solubility, or by any other standard technique for the purification of proteins. The functional properties are evaluated using any suitable assay.

Alternatively, once a Tcl-1b, Tng1 or Tng2 protein produced by a recombinant is identified, the amino acid sequence of the protein is deduced from the nucleotide sequence of the chimeric gene contained in the recombinant. As a result, the protein is synthesized by standard chemical methods known in the art (e.g., see Hunkapiller, et al., 1984, Nature, 310:105-111).

In a specific embodiment of the present invention, such Tcl-1b, Tng1 or Tng2 proteins, whether produced by recombinant DNA techniques or by chemical synthetic methods, include, but are not limited to, those containing, as a primary amino acid sequence, all or part of the amino acid sequence, substantially, as in SEQ. ID. NO: 39, 42, and 44, respectively, as well as fragments and other derivatives; and analogs thereof.

Generation of Antibodies to Tcl-1b, Tng1 or Tng2

According to the invention, Tcl-1b, Tng1 or Tng2 protein, its fragments or other derivatives, or analogs thereof, are used as an immunogen to generate antibodies which recognize such an immunogen. Such antibodies include but are not limited to polyclonal, monoclonal, chimeric, single chain, Fab fragments, and an Fab expression library. In a specific embodiment, antibodies to a human Tcl-1b, Tng1 or Tng2, respectively, protein are produced.

Various procedures known in the art are used for the production of polyclonal antibodies to a Tcl-1b, Tng1 or Tng2 protein or derivative or analog. For the production of antibody, various host animals are immunized by injection with the native Tcl-1b, Tng1 or Tng2 protein, or a synthetic version, or derivative (e.g., fragment) thereof, including but not limited to rabbits, mice, rats, etc. Various adjuvants are used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

In a specific example, the entire protein product of the TCL-1b, TNG1 or TNG2 gene expressed in bacteria was used to immunize rabbits against Tcl-1b, Tng1 or Tng2, respectivley. Such antibodies recognized the Tcl-1b, Tng1 or Tng2 protein, respectively, in a variety of leukemia and lymphoma cells by Western Blot and by immunoprecipitation.

For preparation of monoclonal antibodies directed toward a Tcl-1b, Tng1 or Tng2 protein sequence (SEQ. ID. NO: 39, 42, 44, respectively) or analog thereof, any technique which provides for the production of antibody molecules by continuous cell lines in culture are used. For example, the hybridoma technique originally developed by Kohler and Milstein (1975, Nature, 256:495-497), as well as the trioma technique, the human B-cell hybridoma technique (Kozbor et al., 1983, Immunology Today, 4:72), an the EBV hybridoma technique to produce human monoclonal antibodies (Cole, et al., 1985, in Monoclonal Antibodies and Cancer Therapy, Alan R. Liss, Inc., pp. 77-96). In an additional embodiment of the invention, monoclonal antibodies are produced in germ-free animals utilizing recent technology (PCT/US90/02545). According to the invention, human antibodies are used and are obtained by using human hybridomas (Cote, et al., 1983, Proc Natl Acad Sci USA, 80:2026-2030) or by transforming human B cells with EBV virus in vitro (Cole et al., 1985, in Monoclonal Antibodies and Cancer Therapy, Alan R. Liss, pp. 77-96). In fact, according to the invention, techniques developed for the production of “chimeric antibodies” (Morrison, et al., 1984, Proc Natl Acad Sci USA, 81:6851-6855; Neuberger, et al., 1984, Nature, 312:604-608; Takeda et al., 1985, Nature, 314:452-454) by splicing the genes from a mouse antibody molecule specific for Tcl-1b, Tng1 or Tng2 proteins together with genes from a human antibody molecule of appropriate biological activity is used; such antibodies are within the scope of this invention.

According to the invention, techniques described for the production of single chain antibodies (U.S. Pat. No. 4,946,778) are adapted to produce Tcl-1b, Tng1 or Tng2-specific single chain antibodies. An additional embodiment of the invention utilizes the techniques described for the construction of Fab expression libraries (Huse, et al., 1989, Science, 246:1275-1281) to allow rapid and easy identification of monoclonal Fab fragments with the desired specificity for Tcl-1b, Tng1 or Tng2 proteins, derivatives, or analogs.

Antibody fragments which contain the idiotype of the molecule are generated by known techniques. For example, such fragments include, but are not limited to,: the F(ab′).sub.2 fragment which is produced by pepsin digestion of the antibody molecule; the Fab′ fragments which are generated by reducing the disulfide bridges of the F(ab′).sub.2fragment, and the Fab fragments which are generated by treating the antibody molecule with papain and a reducing agent.

In the production of antibodies, screening for the desired antibody is accomplished by techniques known in the art, e.g. ELISA (enzyme-linked immunosorbent assay). For example, to select antibodies which recognize a specific domain of a Tcl-1b, Tng1 or Tng2 protein, one assays the generated hybridomas for a product which binds to a Tcl-1b, Tng1 or Tng2, respectively, fragment containing such domain. For selection of an antibody specific to human Tcl-1b, Tng1 or Tng2, one selects on the basis of positive binding to human Tcl-1b, Tng1 or Tng2, respectively, and a lack of binding to, for example, mouse Tcl-1b, Tng1 or Tng2.

The foregoing antibodies are used in methods known in the art relating to the localization and activity of the protein sequences of the invention, e.g., for imaging these proteins, measuring levels thereof in appropriate physiological samples, etc.

Structure of the Tcl-1b, Tng1 and Tng2 Gene and Protein

The structure of the Tcl-1b, Tng1 and Tng2 gene and protein is analyzed by to various methods known in the art.

Genetic Analysis

The cloned DNA or cDNA corresponding to the TCL-1b, TNG1 or TNG2 gene is analyzed by methods including but not limited to Southern hybridization (Southern, E. M., 1975, J Mol Biol, 98:503-517), Northern hybridization (see, e.g., Freeman, et al., 1983, Proc Natl Acad Sci USA, 80:4094-4098), restriction endonuclease mapping (Maniatis, T., 1982, Molecular Cloning, A Laboratory, Cold Spring Harbor, N.Y.), and DNA sequence analysis. Polymerase chain reaction (PCR; U.S. Pat. Nos. 4,683,202, 4,683,195, and 4,889,818; Proc Natl Acad Sci USA 85:7652-7656; Ochman, et al., 1988, Genetics, 120:621-623; Loh, et al., 1989, Science, 243:217-220) followed by Southern hybridization with a TCL-1b, TNG1 or TNG2 specific probe allows the detection of the TCL-1b, TNG1 or TNG2 gene, respectively, in DNA from various cell types. In one embodiment, Southern hybridization is used to determine the genetic linkage of TCL-1b, TNG1 or TNG2, respectively. PCR followed by hybridization assay is also used to detect or measure TCL-1b, TNG1 or TNG2 RNA, respectively, or 14q32.1 chromosomal abnormalities. Northern hybridization analysis is used to determine the expression levels of the TCL-1b, TNG1 or TNG2 gene. Various cell types, at various states of development or activity are tested for TCL-1b expression. The stringency of the hybridization conditions for both Southern and Northern hybridization, or dot blots, are manipulated to ensure detection of nucleic acids with the desired degree of relatedness to the specific TCL-1b, TNG1 or TNG2 probe respectively, used.

Restriction endonuclease mapping is used to roughly determine the genetic structure of the TCL-1b, TNG1 or TNG2 gene. Restriction maps derived by restriction endonuclease cleavage are confirmed by DNA sequence analysis.

DNA sequence analysis is performed by any techniques known in the art, including, but not limited to, the method of Maxam and Gilbert (1980, Meth Enzymol, 65:499-560), the Sanger dideoxy method (Sanger, et al., 1977, Proc Natl Acad Sci USA, 74:5463), the use of T7 DNA polymerase (Tabor and Richardson, U.S. Pat. No. 4,795,699), or use of an automated DNA sequenator (e.g., Applied Biosystems, Foster City, Calif.). The cDNA sequence of a representative TCL-1b, TNG1 or TNG2 gene comprises the sequence substantially as disclosed herein (SEQ. ID. NO: 38, 41 and 43, respectively).

Protein Analysis

The amino acid sequence of the Tcl-1b, Tng1 and Tng2 protein are derived by deduction from the DNA sequence, or alternatively, by direct sequencing of the protein, e.g., with an automated amino acid sequencer. The amino acid sequence of a representative Tcl-1b, Tng1 and Tng2 protein comprises the sequence substantially as depicted in SEQ ID NO: 39, 42, and 44, respectively, with the representative mature protein that is shown by amino acid numbers 1-128, 1-141, and 1-110, respectively.

The Tcl-1b, Tng1 and Tng2 protein sequence are further characterized by a hydrophilicity analysis (Hopp, T. and Woods, K., 1981, Proc Natl Acad Sci USA, 78:3824). A hydrophilicity profile is used to identify the hydrophobic and hydrophilic regions of the Tcl-1b, Tng1 or Tng2 protein and the corresponding regions of the gene sequence which encode such regions.

Secondary structural analysis (Chou, P. and Fasman, G., 1974, Biochemistry, 13:222) is also done, to identify regions of the Tcl-1b, Tng1 or Tng2 protein that assume specific secondary structures.

Manipulation, translation, and secondary structure prediction, as well as open reading frame prediction and plotting, is also accomplished using computer software programs available in the art.

Other methods of structural analysis are also employed. These include, but are not limited to, X-ray crystallography (Engstom, A., 1974, Biochem Exp Biol, 11:7-13) and computer modeling (Fletterick, R. and Zoller, M. (eds.), 1986, Computer Graphics and Molecular Modeling, in Current Communications in Molecular Biology, Cold Spring Harbor Laboratory, Cold Spring Harbor, N.Y.).

Uses of TCL-1b, TNG1 or TNG2 and its Tcl-1b Tng1 or Tng2, Respectively, Protein Product and Antibodies Thereto

Chromosomal translocations and inversions associated with the TCL-1b, TNG1 or TNG2 locus on chromosome 14, e.g., t(14:14)(q11;q32) chromosome translocation, inv(14)(q11;q32) chromosome inversion, and t(7:14)(q35:q32) chromosome translocation, are associated with several post-thymic types of T-cell leukemias, including, but not limited to, T-prolymphocytic leukemias (T-PLL) (Brito-Babapulle and Catovsky, 1991, Cancer Genet Cytogenet, 55:1-9), acute and chronic leukemias associated with the immunodeficiency syndrome ataxia-telangiectasia (AT) (Russo et al., 1988, Cell, 53:137-144; Russo et al., 1989, Proc. Natl. Acad. Sci. U.S.A. 86:602-606), and adult T-cell leukemia (Virgilio et al., 1993, PNAS, 90:9275-9279). In some cases of AT-associated translocations, in T-cell leukemia and lymphoma involving the 14q32.1 band, clonal expansion of cells carrying abnormalities in 14q32.1 have been documented in some cases prior to the development of overt malignancy (Russo, et al., 1988 Cell, 53:137-144). Therefore, a TCL-1b, TNG1 or TNG2 polynucleotide, its Tcl-1b, Tng1 or Tng2, respectively, protein product and antibodies thereto are used for diagnostic and/or therapeutic/prophylactic purposes for the above described diseases, as well as other disorders associated with chromosomal translocations and inversions associated with to TCL-1, TNG1 or, TNG2 gene locus and/or, increased expression of TCL-1b, TNG1 or TNG2 RNA or protein, respectively. A TCL-1b, TNG1 or TNG2 polynucleotide, its encoded protein product and antibodies thereto are used for therapeutic/prophylactic purposes alone or in combination with other therapeutics useful in the treatment of T-cell leukemias. Such molecules are also used in diagnostic assays, such as immunoassays, to detect, prognose, diagnose, or monitor various conditions, diseases, and disorders associated with TCL-1b, TNG1 or TNG2 gene expression or monitor the treatment thereof. Accordingly, in specific embodiments, T-cell malignancies or premalignant changes in such tissues is diagnosed by detecting increased TCL-1b, TNG1 or TNG2 gene expression in patient samples relative to the level of TCL-1b, TNG1 or TNG2 gene expression in an analogous non-malignant sample (from the patient or another person, as determined experimentally or as is known as a standard level in such samples). For diagnostic purposes, a TCL-1b, TNG1 or TNG2 polynucleotide is used to detect TCL-1b, TNG1 or TNG2 gene, respectivley, expression or increased TCL-1b, TNG1 or TNG2 gene expression in disease states, such as, T-cell leukemias and lymphomas. For therapeutic purposes, a Tcl-1b, Tng1 or Tng2 protein is used to make anti-Tcl 1b, anti-Tng1 or anti-Tng2 antibodies that neutralize the activity of Tcl-1b, Tng1 or Tng2, respectively. Included within the scope of the present invention are oligonucleotide sequences, that include antisense RNA and DNA molecules and ribozymes, that function to inhibit expression of a TCL-1b, TNG1 or TNG2 RNA or protein.

Diagnostic Uses

The TCL-1b, TNG1 or TNG2 gene sequence is associated with disease states associated with chromosome 14 translocations and inversions around the TCL-1b, TNG1 or TNG2 gene locus, is preferentially expressed early in T and B lymphocyte differentiation and demonstrates a high level of expression in cells from patients diagnosed with T-PLL carrying an inversion of chromosome 14, inv(14)(q11;q32) or patients carrying a t(14:14)(q11;q32) chromosome translocation. Accordingly, TCL-1b, TNG1 or TNG2 gene sequences (SEQ. ID. NO: 40, 45, and 46, respectively) are used diagnostically for the detection of diseases states resulting from chromosomal abnormalities, e.g., translocations, inversions and deletions, involving the TCL-1b, TNG1 or TNG2 gene locus of chromosome 14. Nucleic acids comprising TCL-1b, TNG1 or TNG2 nucleotide sequences of at least 8 nucleotides, at least 15 nucleotides, at least 25 nucleotides, at least 50 nucleotides, at least 100 nucleotides, at least 200 nucleotides, at least 300 nucleotides, or at least 387 nucleotides up to 1324 nucleotides of SEQ ID NO: 38, 41 and 43 cDNA, respectivley, are used as probes in hybridization assays for the detection and measurement of TCL-1b, TNG1 or TNG2 gene (SEQ. ID. NO: 40, 45, and 46, respectively). Nucleic acids of not more than 5 kilobases, of not more than 10 kilobases, not more than 25 kilobases, not more than 50 kilobases or not more than 70 kilobases which are hybridizable to a TCL-1b, TNG1 or TNG2 gene, cDNA, or complementary strand is used as probes in hybridization assays for the detection and measurement of TCL-1b, TNG1 or TNG2 nucleotide sequences. As an example, the TCL-1b, TNG1 or TNG2 DNA sequence is used in hybridization assays, e.g., Southern or Northern analysis, including in situ hybridization assays, of patient's samples to diagnose abnormalities of TCL-1b, TNG1 or TNG2 gene expression, respectively. Hybridization assays are used to detect, prognose, diagnose, or monitor conditions, disorders, or disease states, such as T-cell malignancies, associated with aberrant changes in TCL-1b, TNG1 or TNG2 expression and/or activity as described supra. In particular, such a hybridization assay is carried out by a method comprising contacting a sample containing nucleic acid with a nucleic acid probe capable of hybridizing to TCL-1b, TNG1 or TNG2 DNA or RNA, under conditions such that hybridization can occur, and detecting or measuring any resulting hybridization. In particular, hybridization assays are used to detect the presence of abnormalities associated with increased expression of TCL-1b, TNG1 or TNG2 mRNA, by hybridizing mRNA or cDNA from a patient sample to a TCL-1b, TNG1 or TNG2, respectively, probe, and measuring the amount of resulting hybridization. For example, assays which are used include, but are not limited to Northern blots, Dot blots, reverse transcriptase PCR, etc. A preferred hybridization assay is Northern blot analysis of a patient sample using TCL-1b, TNG1 or TNG2 gene probes of at least 15 polynucleotides up to the full length cDNA sequence of each respective gene (SEQ. ID. NO: 38, 41 and 43, respectively). Another preferred hybridization assay is in situ hybridization analysis of a patient sample using anti-Tcl-1b, anti-Tng1 or anti-Tng2 antibodies or TCL-1b, TNG1 or TNG2 nucleotide hybridization probes. Such techniques are well known in the art, and are in fact the basis of many commercially available diagnostic kits.

As used herein, patient samples which are used include, but are not limited to, fresh or frozen tissue samples, which are used in in situ hybridization assays; cell or tissue samples containing T-lymphocytes and, in general, patient samples containing nucleic acid, such as peripheral blood lymphocytes (PBL) and T-lymphocytes which are used in assays that measure or quantitate TCL-1b, TNG1 or TNG2 nucleic acid.

Polynucleotide sequences of TCL-1b, TNG1 or TNG2 consisting of at least 8 to 25 nucleotides that are useful as primers in primer dependent nucleic acid amplification methods are used for the detection of TCL-1b, TNG1 or TNG2, resepectively, gene sequences in patient samples. Primer dependent nucleic acid amplification methods useful in the present invention include, but are not limited to, polymerase chain reaction (PCR), competitive PCR, cyclic probe reaction, and ligase chain reaction. Such techniques are well known by those of skill in the art. A preferred nucleic acid amplification method of the present invention is reverse transcriptase PCR (RT-PCR) (Siebert, et al., 1992, Nature, 359:557-558).

In a particular embodiment of the present invention, each primer of a pair of primers for use in a primer dependent nucleic aid amplification method is selected from a different exon of the genomic TCL-1b, TNG1 or TNG2 nucleotide sequences. For example, if one primer of a pair or primers is selected from exon 1 of the TCL-1b, TNG1 or TNG2 genomic sequence, the second primer will be selected from exon 2, 3 or 4 of the TCL-1b or TNG2, respectively, or exon 2 of the TNG1 genomic sequence. As another example, if one primer of a pair of primers is selected from exon 2 of the TCL-1b or TNG2 genomic sequence, the second primer will be selected from exon 1, 3, or 4 of the TCL-1b TNG2 genomic sequence, respectively. By selecting each primer of a pair of primers for use in a primer dependent nucleic acid amplification method from a different exon, amplified genomic nucleotide sequences are distinguished from amplified cDNA nucleotide sequences due to the size difference of the resulting amplified sequences. Resulting amplified genomic nucleotide sequences will contain amplified intron sequences and will be of a larger size than amplified cDNA nucleotide sequences that will not contain amplified intron sequences. For amplification of cDNA nucleotide sequences, the primer sequences should be selected from exons sequences that are sufficiently far enough apart to provide a detectable amplified nucleotide sequence.

The TCL-1b, TNG1 or TNG2 gene sequences of the present invention (SEQ. ID. NO: 40, 45, and 46, respectively) are used diagnostically for the detection of chromosome 14 abnormalities, in particular translocations t(14:14)(q11:q32) and inv(14)(q11;q32) inversion at 14q32.1. Accordingly, the present invention provides a process for detecting a target sequence indicative of or including a chromosome 14 abnormality in a sample, comprising the steps of amplifying the target sequence in the sample using a first primer of 8 to 25 nucleotides, preferably 18-25 nucleotides, complementary to the nucleotide sequence of SEQ ID NO: 40 (TCL-1b), 45 (TNG1) or 46 (TNG2) or SEQ ID NO: 38 (TCL-1b), 41 (TNG1), or 44 (TNG2) and a second primer complementary to a region teleomeric or centromeric to the TCL-1b, TNG1 or TNG2 gene, respectively, and detecting any resulting amplified target sequence in which the presence of the amplified target sequence is indicative of the abnormality. The present invention also provides a method of diagnosing a T-cell malignancy associated with chromosome 14 abnormalities in a patient by detecting a chromosome 14 abnormality according to the method above in which the presence of the amplified target sequence indicates the presence of a T-cell malignancy in the patient. The resultant amplified target sequence is detected on gel electrophoresis and compared with a normal sample or standard that does not contain a chromosome 14 abnormality. Virgilio et al., supra, disclose polynucleotide sequences useful as second primers. Other polynucleotide sequences useful as second primers are selected from the T-cell receptor .α/δ locus, the T-cell receptor β. chain, or if the chromosome 14 abnormality involves aninversion, a polynucleotide sequence 5′ to exon 1 of the TCL-1b, TNG1 or TNG2 gene, or if the chromosome abnormality involves a translocation, a polynucleotide sequence 3′ to the 3′ intron of the TCL-1b, TNG1 or TNG2 gene. The amplification of genomic DNA target sequences may require generating long PCR products. PCR techniques for generating long PCR products are described in Science (1994) 263: 1564-1565; PCR kits for generating long PCR products are available from Perkin Elmer and Takara Shuzo Co., Ltd. The present invention also provides a method for detecting a target nucleotide sequence indicative of or including at least a portion of a chromosome 14 abnormality in a nucleic acid sample, comprising the steps of hybridizing the sample with a nucleic acid probe of not more than 10 kilobases, comprising in the range of 15-1324 nucleotides complementary to at least a portion of the nucleotide sequence of SEQ ID NO: 40 (TCL-1b), 45 (TNG1) or 46 (TNG2) and detecting or measuring the amount of any resulting hybridization between the probe and the target sequence within the sample. The resultant hybridization between the probe and the target sequence within the sample is detected using gel electrophoresis and is compared to a target sequence from a normal sample or standard that does not contain a chromosome 14 abnormality. The present invention also provides a method of diagnosing a T-cell malignancy associated with chromosome 14 abnormalities in a patient comprising, detecting said chromosome 14 abnormality according to the method above in which the presence of the amplified target sequence indicates the presence of a T-cell malignancy in the patient. Absolute complementarity between a hybridization probe and a target sequence, although preferred, is not required. A sequence “complementary to at least a portion of”, as referred to herein, means a sequence having sufficient complementarity to be able to hybridize with the nucleic acid, forming a stable hybridization complex. The ability to hybridize will depend on both the degree of complementarity and the length of the nucleic acid. Generally, the longer the hybridizing nucleic acid, the more base mismatches with a TCL-1b, TNG1 or TNG2 RNA it may contain and still form a stable duplex (or triplex, as the case is). One skilled in the art can ascertain a tolerable degree of mismatch by use of standard procedures to determine the melting point of the hybridized complex.

An additional aspect of the present invention relates to diagnostic kits for the detection or measurement of TCL-1b, TNG1 or TNG2 gene sequences and Tcl-1b, Tng1 or Tng2, respectively, protein. Accordingly, the present invention provides a diagnostic kit comprising, in a container a compound comprising a probe of not more than 10 kilobases and comprising in the range of 15-1324 nucleotides of the nucleotide sequence of SEQ ID NO. 38 (TCL-1b), 41 (TNG1) or 43 (TNG2) or its complement. Alternatively, the present invention provides a diagnostic kit comprising, in one or more containers, a pair of primers of at least 8-25 nucleotides in which at least one of the primers is hybridizable to SEQ ID NO: 38 (TCL-1b), 41 (TNG1) or 43 (TNG2) or its complement and wherein the primers are capable of priming cDNA synthesis in an amplification reaction. The present invention also provides a diagnostic kit in which at least one of the primers is hybridizable to SEQ ID NO: 38 (TCL-1b), 41 (TNG1) or 43 (TNG2) or its complement and in which one of the primers is hybridizable to a DNA sequence located telomeric or centromeric to the TCL-1b, TNG1 or TNG2 gene. In a specific embodiment, one of the foregoing compounds of the container is detectably labeled.

The amplification reaction of the present invention are a polymerase chain reaction, competitive PCR and competitive reverse-transcriptase PCR (Clementi, et al., 1994, Genet Anal Tech Appl, 11(1):1-6; Siebert et al., 1992, Nature, 359:557-558); cyclic probe reaction, which allows for amplification of a target sequence using a hybrid RNA/DNA probe and RNase (ID Biomedical); ligase chain reaction (Wu, et al., 1989, Genomics, 4:560-569). In a particular embodiment, the chromosomal abnormality associated with a TCL-1b, TNG1 or TNG2 locus is detected as described in PCT Publication No. WO/92/19775, dated Nov. 12, 1992. In a specific embodiment, the TCL-1b, TNG1 or TNG2 probe used in a hybridization assay is detectably labeled. Such a label is any known in the art including, but not limited to, radioactive labels, fluorescent labels, biotin, chemiluminescent labels, etc.

In a specific embodiment in which the assay used employs primers, at least one primer is detectably labeled. In another embodiment, one of a primer pair is attached to a moiety providing for capture, e.g., a magnetic bead.

Anti-Tcl-1b, anti-Tng1 or anti-Tng2 antibodies are generated and used diagnostically to detect the presence of Tcl-1b, Tng1 or Tng2 protein product, respectively, in patient samples thereby identifying disease states associated with chromosome 14 abnormalities. For detection of Tcl-1b, Tng1 or Tng2 protein sequences (SEQ. ID. NO: 39, 42, or 44, respectively), a diagnostic kit of the present invention comprises, in one or more containers, an anti-Tcl-1b, anti-Tng1 or anti-Tng2 antibody which optionally is detectably labeled. In a different embodiment, the kit can comprise in a container, a labeled specific binding portion of an antibody. As used herein, the term detectable label refers to any label which provides directly or indirectly a detectable signal and includes, for example, enzymes, radiolabelled molecules, fluorescent molecules, particles, chemiluminesors, enzyme substrates or cofactors, enzyme inhibitors, or magnetic particles. Examples of enzymes useful as detectable labels in the present invention include alkaline phosphatase and horse radish peroxidase. A variety of methods are available for linking the detectable labels to proteins of interest and includee, for example, the use of a bifunctional agent, such as, 4,4′-difluoro-3,3′-dinitro-phenylsulfone, for attaching an enzyme, for example, horse radish peroxidase, to a protein of interest. The attached enzyme is then allowed to react with a substrate yielding a reaction product which is detectable. The present invention provides a method for detecting a Tcl-1b, Tng1 or Tng2 protein in a patient sample, comprising, contacting the patient sample with an anti-Tcl-1b, anti-Tng1 or anti-Tng2 antibody, respectively, under conditions such that immunospecific binding occurs, and detecting or measuring the amount of any immunospecific binding by the antibody.

Samples are any sample from a patient containing Tcl-1b, Tng1 or Tng2 protein, e.g., tissue sections, peripheral blood lymphocytes, etc In diagnosing disease states, the functional activity of Tcl-1b, Tng1 or Tng2 proteins, derivatives and analogs are assayed by various methods. Accordingly, the present invention also provides a method of diagnosing a T-cell malignancy associated with chromosome 14 abnormalities in a patient comprising, detecting increased expression of Tcl-1b, Tng1 or Tng2 protein in a sample from the patient, in which an increase in Tcl-1b, Tng1 or Tng2, respectivley, protein relative to the level found in such an analogous sample from a normal individual, indicates the presence of a T-cell malignancy in the patient.

For example, in one embodiment, where one is detecting or measuring Tcl-1b, Tng1 or Tng2 protein by assaying for binding to anti-Tcl-1b, anti-Tng1 or anti-Tng2 antibody, respectively, various immunoassays known in the art are used, including, but not limited to, competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in situ immunoassays (using colloidal gold, enzyme or radioisotope abels, for example), western blots, in situ hybridizations, precipitation reactions, agglutination ssays (e.g., gel agglutination assays, hemagglutination assays), complement fixation assays, mmunofluorescence assays, protein A assays, and immunoelectrophoresis assays, etc. In one mbodiment, antibody binding is detected by detecting a label on the primary antibody. In nother embodiment, the primary antibody is detected by detecting binding of a secondary antibody or reagent to the primary antibody. In a further embodiment, the secondary antibody is labelled. Many means are known in the art for detecting binding in an immunoassay and are within the scope of the present invention. In particular, such an immunoassay is carried out by a method comprising contacting a sample derived from a patient with an anti-Tcl-1b, anti-Tng1 or anti-Tng2 antibody under conditions such that immunospecific binding occurs, and detecting or measuring the amount of any immunospecific binding by the antibody. In a specific embodiment, antibody to a Tcl-1b, Tng1 or Tng2 protein is used to assay a patient tissue or serum sample for the presence of a Tcl-1b, Tng1 or Tng2 protein, respectively, where an increased level of Tcl-1b, Tng1 or Tng2 protein is an indication of a diseased condition. In one embodiment of the present invention, the Tcl-1b, Tng1 or Tng2 protein is detected or measured by immunocytochemistry of a patient sample. In another embodiment, assays to measure the levels of Tcl-1b, Tng1 or Tng2 protein or RNA is used to moniter therapy of disease associated with increased expression of Tcl-1b. For example, a decrease in levels of TCL-1b, TNG1 or TNG2 RNA or protein after therapy, relative to the level found before therapy, are indicative of a favorable response to therapy. An increase in such levels after therapy are indicative of a poor response to therapy.

In another embodiment, the levels of Tcl-1b, Tng1 or Tng2 protein or RNA expression are used to stage disease, with an increase in Tcl-1b, Tng1 or Tng2 protein or RNA, respectively, expression indicating disease progression.

Other methods will be known to the skilled artisan and are within the scope of the invention.

Therapeutic/Prophylactic Uses

Inhibitors of Tcl-1b, Tng1 or Tng2 are used therapeutically for the treatment of disease states associated with chromosome 14 abnormalities, in particular at 14q32.1, and/or increased expression of Tcl-1b, Tng1 or Tng2 protein, respectively. In an embodiment of the present invention, a Tcl-1b, Tng1 or Tng2 protein and/or cell line that expresses a Tcl-1b, Tng1 or Tng2 protein, respectively, is used to screen for antibodies, peptides, or other molecules that bind to the Tcl-1b, Tng1 or Tng2 protein and thus may act as agonists or antagonists of Tcl-1b, Tng1 or Tng2 protein. For example, anti-Tcl-1b, anti-Tng1 or anti-Tng2 antibodies capable of neutralizing the activity of a Tcl-1b, Tng1 or Tng2 protein, respectively, are used to inhibit or prevent a disease state associated with chromosome 14 abnormalities and/or expression of Tcl-1b, Tng1 or Tng2 protein, such as T-cell leukemia and lymphoma. Accordingly, the present invention provides a method for treating a disease state associated with a chromosome 14 abnormality in mammal suffering from a disease state associated with a chromosome 14 abnormality comprising, administering a therapeutically effective amount of an anti-Tcl-1b, anti-Tng1 or anti-Tng2 antibody to a mammal suffering from a disease state associated with a chromosome 14 abnormality. Alternatively, screening of organic or peptide libraries with recombinantly expressed Tcl-1b, Tng1 or Tng2 protein are useful for identification of therapeutic molecules that function to inhibit the activity of Tcl-1b, Tng1 or Tng2 protein, respectively. Synthetic and naturally occurring products are screened in a number of ways deemed routine to those of skill in the art.

The ability of antibodies, peptides or other molecules to modulate the effect of Tcl-1b, Tng1 or Tng2 protein on disease states is monitored. For example, the expression of TCL-1b, TNG1 or TNG2 gene sequences (SEQ. ID. NO: 40, 45, or 46, respectively) or Tcl-1b, Tng1 or Tng2 protein sequences (SEQ. ID. NO: 38, 42, or 44, respectively) are detected as described, supra, both before and after administration of a therapeutic composition comprising a TCL-1b, TNG1 or TNG2 nucleotide sequence, Tcl-1b, Tng1 or Tng2 protein sequence, derivative or analog thereof, or antibody thereto, respectively, of the present invention.

A TCL-1b, TNG1 or TNG2 polynucleotide is useful in the treatment of various disease states associated with chromosome 14 abnormalities, such as T-cell leukemias and lymphomas, and/or increased expression of Tcl-1b, Tng1 or Tng2 protein. By introducing TCL-1b, TNG1 or TNG2 antisense gene sequences into cells, gene therapy is used to treat conditions associated with over-expression of TCL-1b, TNG1 or TNG2 genes, respectively. Accordingly, the present invention provides a method for treating a disease state associated with a chromosome 14 abnormality in mammal suffering from a disease state associated with a chromosome 14 abnormality comprising, administering a therapeutically effective amount of a TCL-1b, TNG1 or TNG2 antisense molecule to a mammal suffering from a disease state associated with a chromosome 14 abnormality.

Oligonucleotide sequences, that include antisense RNA and DNA molecules and ribozymes that function to inhibit the translation of a TCL-1b, TNG1 or TNG2 mRNA are within the scope of the invention. “Antisense” as used herein refers to a nucleic acid capable of hybridizing to a portion of a TCL-1b, TNG1 or TNG2 RNA (preferably mRNA) by virtue of some sequence complementarity. Antisense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions of a TCL-1b, TNG1 or TNG2 nucleotide sequence, are preferred. The present invention provides for an antisense molecule comprising a nucleotide sequence complementary to at least a part of the coding sequence of a Tcl-1b, Tng1 or Tng2 protein which is hybridizable to a TCL-1b, TNG1 or TNG2 mRNA, respectively. The present invention also provides for an antisense molecule with a nucleotide sequence complementary to at least a part of the non-coding sequence (SEQ ID NO: 40, 45, or 46, respectively) which hybridizes to the TCL-1b, TNG1 or TNG2 coding sequence (SEQ ID NO: 40, 45, or 46, respectively). In a preferred embodiment of the present invention, the antisense gene sequence is derived from the 5′ noncoding sequence of a TCL-1b, TNG1 or TNG2 gene. In a particularly preferred embodiment of the present invention, the antisense gene sequence is derived from TCL-1b, TNG1 or TNG2 gene (SEQ ID NO: 38, 41, or 43, respectively).

Ribozymes are enzymatic RNA molecules capable of catalyzing the specific cleavage of RNA. The mechanism of ribozyme action involves sequence specific hybridization of the ribozyme molecule to complementary target RNA, followed by a endonucleolytic cleavage. Within the scope of the invention are engineered hammerhead motif ribozyme molecules that specifically and efficiently catalyze endonucleolytic cleavage of TCL-1b, TNG1 or TNG2 RNA sequences.

Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site are evaluated for predicted structural features, such as secondary structure that may render the oligonucleotide sequence unsuitable. The suitability of candidate targets may also be evaluated by testing their accessibility to hybridization with complementary oligonucleotides, using ribonuclease protection assays.

Both anti-sense RNA and DNA molecules and ribozymes of the invention are prepared by any method known in the art for the synthesis of RNA molecules. These include techniques for chemically synthesizing oligodeoxyribonucleotides well known in the art such as for example solid phase phosphoramidite chemical synthesis. Alternatively, RNA molecules are generated by in vitro and in vivo transcription of DNA sequences encoding the antisense RNA molecule. Such DNA sequences are incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, is introduced stably into cell lines.

Various modifications to the DNA molecules are introduced as a means of increasing intracellular stability and half-life. Examples of modifications include, but are not limited to, the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

Methods for introducing nucleic acid into cells or tissue include methods for in vitro introduction of nucleic acid such as the insertion of naked nucleic acid, i.e., by injection into tissue, the introduction of a nucleic acid in a cell ex vivo, the use of a vector such as a virus, retrovirus, phage or plasmic, etc. or techniques such as electroporation which are used in vivo or ex vivo.

Other methods will be known to the skilled artisan and are within the scope of the invention.

Demonstration of Therapeutic or Prophylactic Utility

The TCL-1b, TNG1 or TNG2 polynucleotides, Tcl-1b, Tng1 or Tng2 protein products, respectivley, derivatives and analogs thereof, and antibodies thereto, of the invention are tested in vivo for the desired therapeutic or prophylactic activity. For example, such compounds are tested in suitable animal model systems prior to testing in humans, including but not limited to rats, mice, chicken, cows, monkeys, rabbits, etc. For in vivo testing, prior to administration to humans, any animal model system known in the art are used.

Therapeutic/Prophylactic Methods and Compositions

The invention provides methods of treatment and prophylaxis by administration to a subject of an effective amount of a Therapeutic, i.e., a TCL-1b, TNG1 or TNG2 polynucleotide, Tcl-1b, Tng1 or Tng2 protein, respectively, derivative or analog thereof, or antibody thereto of the present invention. In a preferred aspect, the Therapeutic is substantially purified. The subject is preferably an animal, including but not limited to animals such as cows, pigs, chickens, etc., and is preferably a mammal, and most preferably human.

Various delivery systems are known and used to administer a Therapeutic of the invention, e.g., encapsulation in liposomes, microparticles, microcapsules, expression by recombinant cells, receptor-mediated endocytosis (see, e.g., Wu and Wu, 1987, J Biol Chem, 262:4429-4432), construction of a therapeutic nucleic acid as part of a retroviral or other vector, etc. Methods of introduction include but are not limited to intradermal, intramuscular, intraperitoneal, intravenous, subcutaneous, intranasal, and oral routes. The compounds are administered by any convenient route, for example by infusion or bolus injection, by absorption through epithelial or mucocutaneous linings (e.g., oral mucosa, rectal and intestinal mucosa, etc.) and are administered together with other biologically active agents. Administration is systemic or local. In addition, it are desirable to introduce the pharmaceutical compositions of the invention into the central nervous system by any suitable route, including intraventricular and intrathecal injection; intraventricular injection are facilitated by an intraventricular catheter, for example, attached to a reservoir, such as an Ommaya reservoir.

In a specific embodiment, it are desirable to administer the pharmaceutical compositions of the invention locally to the area in need of treatment; this are achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration is by direct injection at the site (or former site) of a malignant tumor or neoplastic or pre-neoplastic tissue.

In a specific embodiment where the Therapeutic is a nucleic acid encoding a protein therapeutic, the nucleic acid is administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting, agents, or by administering it in linkage to a homeobox-like peptide which is known to enter the nucleus (see e.g., Joliot, et al., 1991, Proc Natl Acad Sci USA, 88:1864-1868), etc. Alternatively, a nucleic acid therapeutic is introduced intracellularly and incorporated within host cell DNA for expression, by homologous recombination.

The present invention also provides pharmaceutical compositions. Such compositions comprise a therapeutically effective amount of a therapeutic, and a pharmaceutically acceptable carrier or excipient. Such a carrier includes but is not limited to saline, buffered saline, dextrose, water, glycerol, ethanol, and combinations thereof. The carrier and composition are sterile. The formulation should suit the mode of administration.

The composition, if desired, can also contain minor amounts of wetting or emulsifying agents, or pH buffering agents. The composition is a liquid solution, suspension, emulsion, tablet, pill, capsule, sustained release formulation, or powder. The composition is formulated as a suppository, with traditional binders and carriers such as triglycerides. Oral formulation includes standard carriers such as pharmaceutical grades of mannitol, lactose, starch, magnesium stearate, sodium saccharine, cellulose, magnesium carbonate, etc.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it is dispensed with an infusion bottle containing sterile pharmaceutical grade water or saline. Where the composition is administered by injection, an ampoule of sterile water for injection or saline is provided so that the ingredients are mixed prior to administration.

The Therapeutics of the invention are formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the Therapeutic of the invention which will be effective in the treatment of a particular disorder or condition will depend on the nature of the disorder or condition, and can be determined by standard clinical techniques. In addition, in vitro assays are employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses are extrapolated from dose-response curves derived from in vitro or animal model test systems.

Suppositories generally contain active ingredient in the range of 0.5% to 10 k by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) is a notice in the form prescribed by a governmental agency regulating the manufacture, use or sale of pharmaceuticals or biological products, which notice reflects approval by the agency of manufacture, use or sale for human administration.

Antisense Regulation of TCL-1b, TNG1 and TNG2 Gene Expression

The present invention provides the therapeutic or prophylactic use of nucleic acids of at least six nucleotides that are antisense to a TCL-1b, TNG1 or TNG2 gene (SEQ. ID. NO: 40, 45, or 46, respectively) or TCL-1b, TNG1 or TNG2 cDNA (SEQ. ID. NO: 38, 41, or 43, respectively) encoding Tcl-1b, Tng1 or Tng2 (SEQ. ID. NO 39, 42 or 44), respectively, or a portion thereof. Such antisense nucleic acids have utility as Antagonist Therapeutics of the invention, and is used in the treatment or prevention of disorders, e.g., T-cell malignancies as described supra.

The antisense nucleic acids of the invention are oligonucleotides that are double-stranded or single-stranded, RNA or DNA or a modification or derivative thereof, which can be directly administered to a cell, or which are produced intracellularly by transcription of exogenous, introduced sequences.

In a specific embodiment, the TCL-1b, TNG1 or TNG2 antisense polynucleotides provided by the instant invention can be used for the treatment of disease states associated with chromosome 14 abnormalities, in particular at 14q32.1, wherein the disease state can be demonstrated (in vitro or in vivo) to express the TCL-1b, TNG1 or TNG2 gene, respectively. Such demonstration can be by detection of TCL-1b, TNG1 or TNG2 RNA or of Tcl-1b, Tng1 or Tng2 protein, respectively.

The invention further provides pharmaceutical compositions comprising an effective amount of the TCL-1b, TNG1 or TNG2 antisense nucleic acids of the invention in a pharmaceutically acceptable carrier, as described supra. Methods for treatment and prevention of disease states associated with chromosome 14, such as to T-cell malignancies comprising administering the pharmaceutical compositions of the invention are also provided.

In another embodiment, the invention is directed to methods for inhibiting the expression of a TCL-1b, TNG1 or TNG2 nucleic acid sequence in a prokaryotic or eukaryotic cell comprising providing the cell with an effective amount of a composition comprising an antisense TCL-1b, TNG1 or TNG2 nucleic acid, respectively, of the invention.

The TCL-1b, TNG1 or TNG2 antisense polynucleotides are of at least six nucleotides and are preferably oligonucleotides (ranging from 6 to about 50 oligonucleotides). In specific aspects, the oligonucleotide is at least 10 nucleotides, at least 20 nucleotides, at least 30 nucleotides, or at least 40 nucleotides. The oligonucleotides are DNA or RNA or chimeric mixtures or derivatives or modified versions thereof, single-stranded or double-stranded. The oligonucleotide is modified at the base moiety, sugar moiety, or phosphate backbone. The oligonucleotide may include other appending groups such as peptides, or agents facilitating transport across the cell membrane (see, e.g., Letsinger, et al., 1989, Proc Natl Acad Sci USA, 86:6553-6556; Lemaitre, et al., 1987, Proc Natl. Acad Sci USA, 84:648-652; PCT Publication No. WO 88/09810, published Dec. 15, 1988) or blood-brain barrier (see, e.g., PCT Publication No. WO 89/10134, published Apr. 25, 1988), hybridization-triggered cleavage agents (see, e.g., Krol, et al., 1988, BioTechniques, 6:958-976) or intercalating agents (see, e.g., Zon, 1998, Pharm Res 5:539-549).

The oligonucleotide are conjugated to another molecule, e.g., a peptide, hybridization triggered cross-linking agent, transport agent, hybridization-triggered cleavage agent, etc.

Oligonucleotides of the invention are synthesized by standard methods known in the art, e.g., by use of an automated DNA synthesizer (such as are commercially available from Biosearch, Applied Biosystems, etc.). As examples, phosphorothioate oligos are synthesized by the method of Stein et al. (1988, Nucl. Acids Res. 16:3209), methylphosphonate oligos are prepared by use of controlled pore glass polymer supports (Sarin, et al., 1988, Proc Natl Acad Sci USA, 85:7448-7451), etc.

In a specific embodiment, the TCL-1b, TNG1 or TNG2 antisense oligonucleotide comprises catalytic RNA, or a ribozyme (see, e.g., PCT International Publication WO 90/11364, published Oct. 4, 1990; Sarver, et al., 1990, Science, 247:1222-1225). In another embodiment, the oligonucleotide is a 2′-O-methylribonucleotide (Inoue, et al., 1987, Nucl Acids Res, 15:6131-6148), or a chimeric RNA-DNA analogue (Inoue, et al., 1987, FEBS Lett, 215:327-330).

In an alternative embodiment, the TCL-1b, TNG1 or TNG2 antisense nucleic acid of the invention is produced intracellularly by transcription from an exogenous sequence. For example, a vector is introduced in vivo such that it is taken up by a cell, within which cell the vector or a portion thereof is transcribed, producing an antisense nucleic acid (RNA) of the invention. Such a vector would contain a sequence encoding the TCL-1b, TNG1 or TNG2 antisense nucleic acid, respectively. Such a vector can remain episomal or become chromosomally integrated, as long as it is transcribed to produce the desired antisense RNA. Such vectors are constructed by recombinant DNA technology methods standard in the art. Vectors are plasmid, viral, or others known in the art, used for replication and expression in mammalian cells. Expression of the sequence encoding the TCL-1b, TNG1 or TNG2 antisense RNA is by any promoter known in the art to act in mammalian, preferably human, cells. Such promoters are inducible or constitutive. Such promoters include but are not limited to: the SV40 early promoter region (Bernoist and Chambon, 1981, Nature, 290:304-310), the promoter contained in the 3′ long terminal repeat of Rous sarcoma virus (Yamamoto, et al., 1980, Cell, 22:787-797), the herpes thymidine kinase promoter (Wagner, et al., 1981, Proc Natl Acad Sci USA, 78:1441-1445), the regulatory sequences of the metallothionein gene (Brinster, et al., 1982, Nature, 296:3942), etc.

The antisense nucleic acids of the invention comprise a sequence complementary to at least a portion of an RNA transcript of a TCL-1b, TNG1 or TNG2 gene, preferably a human TCL-1b, TNG1 or TNG2 gene (SEQ. ID. NO: 40, 45, or 46, respectively). However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded TCL-1b, TNG1 or TNG2 antisense nucleic acids, a single strand of the duplex DNA may thus be tested, or triplex formation are assayed. The ability to hybridize will depend on both the degree of complementarity and the length of the antisense nucleic acid. Generally, the longer the hybridizing nucleic acid, the more base mismatches with a TCL-1b, TNG1 or TNG2 RNA it may contain and still form a stable duplex (or triplex, as the case are). One skilled in the art can ascertain a tolerable degree of mismatch by use of standard procedures to determine the melting point of the hybridized complex.

The TCL-1b antisense nucleic acids are used to treat (or prevent) T-cell malignancies, of a cell type which has been shown to express TCL-1b, TNG1 or TNG2 RNA. Malignant, neoplastic, and pre-neoplastic cells which are tested for such expression include, but are not limited, to those described supra. In a preferred embodiment, a single-stranded DNA antisense TCL-1b, TNG1 or TNG2 oligonucleotide is used, respectively.

Malignant (particularly, tumor) cell types which express TCL-1b, TNG1 or TNG2 RNA is identified by various methods known in the art. Such methods include but are not limited to hybridization with a TCL-1b, TNG1 or TNG2-specific nucleic acid (e.g., by Northern hybridization, dot blot hybridization, in situ hybridization), observing the ability of RNA from the cell type to be translated in vitro into Tcl-1b, Tng1 or Tng2, respectively. In a preferred aspect, primary tumor tissue from a patient is assayed for TCL-1b, TNG1 or TNG2 expression prior to treatment.

Pharmaceutical compositions of the invention, comprising an effective amount of a TCL-1b, TNG1 or TNG2 antisense nucleic acid in a pharmaceutically acceptable carrier, is administered to a patient having a malignancy which is of a type that expresses TCL-1b, TNG1 or TNG2 RNA.

The amount of TCL-1b, TNG1 or TNG2 antisense nucleic acid which will be effective in the treatment of a particular disease state or condition will depend on the nature of the disease state or condition, and is determined by standard clinical techniques. Where possible, it is desirable to determine the antisense cytotoxicity of the tumor type to be treated in vitro, and then in useful animal model systems prior to testing and use in humans.

In a specific embodiment, pharmaceutical compositions comprising TCL-1b, TNG1 or TNG2 antisense nucleic acids are administered via liposomes, microparticles, or microcapsules. In various embodiments of the invention, it are useful to use such compositions to achieve sustained release of the TCL-1b, TNG1 or TNG2 antisense nucleic acids. In a specific embodiment, it are desirable to utilize liposomes targeted via antibodies to specific identifiable tumor antigens (Leonetti, et al., 1990, Proc Natl Acad Sci USA, 87:2448-2451; Renneisen, et al., 1990, J Biol Chem, 265:16337-16342).

1. An isolated nucleic acid comprising a nucleotide sequence encoding a Tcl-1b protein, wherein said nucleotide sequence is a cDNA sequence.

2. The isolated nucleic acid of claim 1, wherein said nucleotide sequence encodes a human Tcl-1b protein having an amino acid sequence of SEQ ID NO:39 from amino acid number 1 to 128.

3. An isolated nucleic acid of not more than 50 kilobases which contains at least an 18 nucleotide portion encoding a Tcl-1b protein fragment.

4. An isolated nucleic acid of not more than 50 kilobases which contains at least an 18 nucleotide portion of the sequence depicted in SEQ ID NO: 40.

5. The isolated nucleic acid of claim 1, comprising a nucleotide sequence of SEQ ID NO:38 from nucleotide number 1 to 1152.

6. A Tcl-1b protein.

7. The isolated Tcl-1b protein of claim 6, comprising an amino acid sequence of SEQ. ID. NO: 39 from amino acid 1-128.

8. An isolated nucleic acid, comprising a sequence encoding a fragment of a protein having an amino sequence of SEQ ID NO.39 from amino acid number 1 to 128, which fragment can be specifically bound by an antibody to a Tcl-1b protein.

9. A recombinant DNA vector, comprising a nucleotide sequence that encodes a Tcl-1b protein, wherein said nucleotide sequence is a cDNA sequence.

10. A host cell that contains said recombinant DNA vector of claim 7.

11. The recombinant DNA vector of claim 7, wherein the nucleotide sequence encodes a human Tcl-1b protein having an amino acid sequence of SEQ ID NO:39 from amino acid number 1 to 128.

12. An isolated nucleic acid of not more than 50 kilobases which contains at least a 50 nucleotide portion of SEQ ID NO: 40.

13. An isolated nucleic acid that is capable of hybridizing under stringent conditions to a nucleotide sequence that is complementary to the cDNA sequence of SEQ ID NO:38, said nucleic acid containing at least an 25 nucleotide portion of SEQ ID NO:38.

14. An isolated nucleic acid that is capable of hybridizing under stringent conditions to a nucleotide sequence that is complementary to a cDNA sequence that encodes a Tcl-1b protein, which protein has an amino acid sequence of SEQ ID NO:39, and said nucleic acid containing at least an 25 nucleotide portion of SEQ ID NO:38.

15. An antisense molecule, comprising a nucleotide sequence complementary to at least a part of a coding sequence of a Tcl-1b protein, which is hybridizable to a Tcl-1b mRNA.

16. The antisense molecule of claim 15, wherein said nucleotide sequence is complementary to a least a part of the sequence depicted in SEQ. ID. NO: 38.

17. A fusion protein comprising a Tcl-1b protein sequence of at least 10 amino acids linked to a non-Tcl-1b protein sequence.

18. An antibody which binds to an epitope of a Tcl-1b protein.

19. An isolated protein comprising an amino acid sequence having at least 70% amino acid sequence identity to an amino acid sequence depicted in SEQ. ID. NO: 39, over a contiguous sequence of at least 25 amino acids.

20. A method for detecting a target sequence indicative of a chromosome 14 abnormality in a sample, comprising the steps of:
a) amplifying said target sequence in said sample using a first primer of 18 to 25 nucleotides complementary to a TCL-1b nucleotide sequence of SEQ. ID. NO: 38, and a second primer complementary to a region telomeric or centromeric, preferably from a T-cell receptor α/δ locus, to said Tcl-1b gene; and
b) detecting any resulting amplified target sequence in which the presence of said amplified target sequence is indicative of said chromosome 14 abnormality.

21. The method of claim 20, wherein said chromosome 14 abnormality is in a Tcl-1b locus and comprises a t(14:14)(q11:q32) translocation or an inv (14)(q11:q32) inversion.

22. A host cell that contains a recombinant vector comprising a cDNA sequence that encodes a human Tcl-1b protein having the amino acid sequence of SEQ ID NO:39 from amino acid number 1 to 128.

23. A host cell that contains a recombinant vector comprising a nucleic acid that is capable of hybridizing under stringent conditions to a nucleotide sequence that is complementary to a cDNA sequence that encodes a Tcl-1b protein, which protein has the amino acid sequence of SEQ ID NO:39, and said nucleic acid containing at least an 25 nucleotide portion of SEQ ID NO:38.

25. A pharmaceutical composition, comprising said antisense molecule of claim 15 or 16 in a pharmaceutically acceptable carrier.

26. A pharmaceutical composition, comprising said antibody of claim 18 in a pharmaceutically acceptable carrier.

27. A method for detecting a target nucleotide sequence indicative of a chromosome 14 abnormality in a nucleic acid sample, comprising the steps of:
a) hybridizing said sample with a nucleic acid probe of not more than 10 kilobases, comprising in the range of 15-1152 nucleotides complementary to said nucleotide sequence of SEQ. ID. NO: 38; and
b) detecting or measuring an amount of any resulting hybridization between said probe and said target sequence within said sample.

28. The method of claim 27, wherein said chromosome 14 abnormality is in a Tcl-1b locus and comprises a t(14:14)(q11:q32) translocation or an inv (14)(q11:q32)inversion.

29. A method for detecting a Tcl-1b protein in a patient sample, preferably a human sample, comprising:
a) contacting said patient sample with an anti-Tcl-1b antibody under conditions such that immunospecific binding occurs; and
b) detecting or measuring an amount of any immunospecific binding by said antibody.

30. A diagnostic kit, comprising in one or more containers, a pair of primers, each having at least 15-25 nucleotides, in which at least one of said primers is hybridizable to SEQ. ID. NO: 38 or it complement and wherein said primers are capable of priming DNA synthesis in a nucleic acid amplification reaction.

31. A method for treating a disease state associated with a chromosome 14 abnormality in a mammal, preferably a human, suffering from said disease state associated with said chromosome 14 abnormality, comprising administering a therapeutically effective amount of a Tcl-1b antisense molecule or an anti-Tcl-1b antibody to said mammal.

32. The method of claim 31, wherein said disease state comprises a T-cell leukemia or lymphoma and said chromosome 14 abnormality comprises a t(14:14)(q11:q32) translocation or an inv (14)(q11:q32) inversion.

33. An isolated nucleic acid comprising a nucleotide sequence encoding a Tng1 protein, wherein said nucleotide sequence is a cDNA sequence.

34. The isolated nucleic acid of claim 33, wherein said nucleotide sequence encodes a human Tng1 protein having an amino acid sequence of SEQ ID NO:42 from amino acid number 1 to 141

35. An isolated nucleic acid of not more than 50 kilobases which contains at least an 18 nucleotide portion encoding a Tng1 protein fragment.

36. An isolated nucleic acid of not more than 50 kilobases which contains at least an 18 nucleotide portion of the sequence depicted in SEQ ID NO: 45.

37. The isolated nucleic acid of claim 33, comprising a nucleotide sequence of SEQ ID NO:41 from nucleotide number 1 to 1500.

38. A Tng1 protein.

39. The isolated Tng1 protein of claim 38, comprising an amino acid sequence of SEQ. ID. NO: 42 from amino acid 1-141.

40. An isolated nucleic acid, comprising a sequence encoding a fragment of a protein having an amino sequence of SEQ ID NO:42 from amino acid number 1 to 141, which fragment can be specifically bound by an antibody to a Tng1 protein.

41. A recombinant DNA vector, comprising a nucleotide sequence that encodes a Tng1 protein, wherein said nucleotide sequence is a cDNA sequence.

42. A host cell that contains said recombinant DNA vector of claim 39.

43. The recombinant DNA vector of claim 39, wherein the nucleotide sequence encodes a human Tng1 protein having an amino acid sequence of SEQ ID NO:42 from amino acid number 1 to 141.

44. An isolated nucleic acid of not more than 50 kilobases which contains at least a 50 nucleotide portion of SEQ ID NO:45.

45. An isolated nucleic acid that is capable of hybridizing under stringent conditions to a nucleotide sequence that is complementary to the cDNA sequence of SEQ ID NO:41, said nucleic acid containing at least an 25 nucleotide portion of SEQ ID NO:41.

46. An isolated nucleic acid that is capable of hybridizing under stringent conditions to a nucleotide sequence that is complementary to a cDNA sequence that encodes a Tng1 protein, which protein has an amino acid sequence of SEQ. ID. NO: 42, and said nucleic acid containing at least an 25 nucleotide portion of SEQ. ID. NO: 41.

47. An antisense molecule, comprising a nucleotide sequence complementary to at least a part of a coding sequence of a Tng1 protein, which is hybridizable to a Tng1 mRNA.

48. The antisense molecule of claim 47, wherein said nucleotide sequence is complementary to a least a part of the sequence depicted in SEQ. ID. NO:41.

49. A fusion protein comprising a Tng1 protein sequence of at least 10 amino acids linked to a non-Tng1 protein sequence.

50. An antibody which binds to an epitope of a Tng1 protien.

51. An isolated protein comprising an amino acid sequence having at least 70% amino acid sequence identity to an amino acid sequence depicted in SEQ. ID. NO: 42, over a contiguous sequence of at least 25 amino acids.

52. A method for detecting a target sequence indicative of a chromosome 14 abnormality in a sample, comprising the steps of:
a) amplifying said target sequence in said sample using a first primer of 18 to 25 nucleotides complementary to a TNG1 nucleotide sequence of SEQ. ID. NO: 41, and a second primer complementary to a region telomeric or centromeric, preferably from a T-cell receptor α/δ locus, to said Tng1 gene; and
b) detecting any resulting amplified target sequence in which the presence of said amplified target sequence is indicative of said chromosome 14 abnormality.

53. The method of claim 52, wherein said chromosome 14 abnormality is in a Tng1 locus and comprises a t(14:14)(q11:q32) translocation or an inv (14)(q11:q32) inversion.

54. A host cell that contains a recombinant vector comprising a cDNA sequence that encodes a human Tng1 protein having the amino acid sequence of SEQ. ID. NO: 42 from amino acid number 1 to 141.

55. A host cell that contains a recombinant vector comprising a nucleic acid that is capable of hybridizing under stringent conditions to a nucleotide sequence that is complementary to a cDNA sequence that encodes a Tng1 protein, which protein has the amino acid sequence of SEQ. ID. NO: 42, and said nucleic acid containing at least an 25 nucleotide portion of SEQ. ID. NO: 41.

56. A pharmaceutical composition, comprising said antisense molecule of claim 47 or 48 in a pharmaceutically acceptable carrier.

57. A pharmaceutical composition, comprising said antibody of claim 50 in a pharmaceutically acceptable carrier.

58. A method for detecting a target nucleotide sequence indicative of a chromosome 14 abnormality in a nucleic acid sample, comprising the steps of:
a) hybridizing said sample with a nucleic acid probe of not more than 10 kilobases, comprising in the range of 15-1500 nucleotides complementary to said nucleotide sequence of SEQ. ID. NO: 41; and
b) detecting or measuring an amount of any resulting hybridization between said probe and said target sequence within said sample.

59. The method of claim 58, wherein said chromosome 14 abnormality is in a Tng1 locus and comprises a t(14:14)(q11:q32) translocation or an inv (14)(q11:q32) inversion.

60. A method for detecting a Tng1 protein in a patient sample, preferably a human sample, comprising:
a) contacting, said patient sample with an anti-Tng1 antibody under conditions such that immunospecific binding occurs; and
b) detecting or measuring an amount of any immunospecific binding by said antibody.

61. A diagnostic kit, comprising in one or more containers, a pair of primers, each having at least 15-25 nucleotides, in which at least one of said primers is hybridizable to SEQ. ID. NO: 41 or it complement and wherein said primers are capable of priming DNA synthesis in a nucleic acid amplification reaction.

62. A method for treating a disease state associated with a chromosome 14 abnormality in a mammal, preferably a human, suffering from said disease state associated with said chromosome 14 abnormality, comprising administering a therapeutically effective amount of a Tng1 antisense molecule or an anti-Tng1 antibody to said mammal.

63. The method of claim 62, wherein said disease state comprises a T-cell leukemia or lymphoma and said chromosome 14 abnormality comprises a t(14:14)(q11:q32) translocation or an inv (14)(q11:q32) inversion.

64. An isolated nucleic acid comprising a nucleotide sequence encoding a Tng2 protein, wherein said nucleotide sequence is a cDNA sequence.

65. The isolated nucleic acid of claim 64, wherein said nucleotide sequence encodes a human Tng2 protein having an amino acid sequence of SEQ. ID. NO: 44 from amino acid number 1 to 110.

66. An isolated nucleic acid of not more than 50 kilobases which contains at least an 18 nucleotide portion encoding a Tng2 protein fragment.

67. An isolated nucleic acid of not more than 50 kilobases which contains at least an 18 nucleotide portion of the sequence depicted in SEQ. ID. NO: 46.

68. The isolated nucleic acid of claim 64, comprising a nucleotide sequence of SEQ ID NO: 43 from nucleotide number 1 to XXX.

69. A Tng2 protein.

70. The isolated Tng2 protein of claim 69, comprising an amino acid sequence of SEQ. ID. NO: 44 from amino acid 1-110.

71. An isolated nucleic acid, comprising a sequence encoding a fragment of a protein having an amino sequence of SEQ. ID. NO:44 from amino acid number 1 to 110, which fragment can be specifically bound by an antibody to a Tng2 protein.

72. A recombinant DNA vector, comprising a nucleotide sequence that encodes a Tng2 protein, wherein said nucleotide sequence is a cDNA sequence.

73. A host cell that contains said recombinant DNA vector of claim 70.

74. The recombinant DNA vector of claim 70, wherein the nucleotide sequence encodes a human Tng2 protein having an amino acid sequence of SEQ ID NO:44 from amino acid number 1 to 110.

75. An isolated nucleic acid of not more than 50 kilobases which contains at least a 50 nucleotide portion of SEQ ID NO:46.

76. An isolated nucleic acid that is capable of hybridizing under stringent conditions to a nucleotide sequence that is complementary to the cDNA sequence of SEQ ID NO:43, said nucleic acid containing at least an 25 nucleotide portion of SEQ ID NO:43.

77. An isolated nucleic acid that is capable of hybridizing under stringent conditions to a nucleotide sequence that is complementary to a cDNA sequence that encodes a Tng2 protein, which protein has an amino acid sequence of SEQ ID NO: 44, and said nucleic acid containing at least an 25 nucleotide portion of SEQ ID NO:43.

78. An antisense molecule, comprising a nucleotide sequence complementary to at least a part of a coding sequence of a Tng2 protein, which is hybridizable to a Tng2 mRNA.

79. The antisense molecule of claim 78, wherein said nucleotide sequence is complementary to a least a part of the sequence depicted in SEQ. ID. NO: 43.

80. A fusion protein comprising a Tng2 protein sequence of at least 10 amino acids linked to a non-Tng2 protein sequence.

81. An antibody which binds to an epitope of a Tng2 protien.

82. An isolated protein comprising an amino acid sequence having at least 70% amino acid sequence identity to an amino acid sequence depicted in SEQ. ID. NO: 44, over a contiguous sequence of at least 25 amino acids.

83. A method for detecting a target sequence indicative of a chromosome 14 abnormality in a sample, comprising the steps of:
a) amplifying said target sequence in said sample using a first primer of 18 to 25 nucleotides complementary to a TNG2nucleotide sequence of SEQ. ID. NO: 43, and a second primer complementary to a region telomeric or centromeric, preferably from a T-cell receptor α/δ locus, to said Tng2 gene; and
b) detecting any resulting amplified target sequence in which the presence of said amplified target sequence is indicative of said chromosome 14 abnormality.

84. The method of claim 83, wherein said chromosome 14 abnormality is in a Tng2 locus and comprises a t(14:14)(q11:q32) translocation or an inv (14)(q11:q32) inversion.

85. A host cell that contains a recombinant vector comprising a cDNA sequence that encodes a human Tng2 protein having the amino acid sequence of SEQ ID NO: 44 from amino acid number 1 to 110.

86. A host cell that contains a recombinant vector comprising a nucleic acid that is capable of hybridizing under stringent conditions to a nucleotide sequence that is complementary to a cDNA sequence that encodes a Tng2 protein, which protein has the amino acid sequence of SEQ ID NO: 44, and said nucleic acid containing at least an 25 nucleotide portion of SEQ ID NO: 43.

87. A pharmaceutical composition, comprising said antisense molecule of claim 78 or 79 in a pharmaceutically acceptable carrier.

88. A pharmaceutical composition, comprising said antibody of claim 80 in a pharmaceutically acceptable carrier.

89. A method for detecting a target nucleotide sequence indicative of a chromosome 14 abnormality in a nucleic acid sample, comprising the steps of:
a) hybridizing said sample with a nucleic acid probe of not more than 10 kilobases, comprising in the range of 15-2000 nucleotides complementary to said nucleotide sequence of SEQ. ID. NO: 43; and
b) detecting or measuring an amount of any resulting hybridization between said probe and said target sequence within said sample.

90. The method of claim 89, wherein said chromosome 14 abnormality is in a Tng2 locus and comprises a t(14:14)(q11:q32) translocation or an inv (14)(q11:q32) inversion.

91. A method for detecting a Tng2 protein in a patient sample, preferably a human sample, comprising:
a contacting said patient sample with an anti-Tng2 antibody under conditions such that immunospecific binding occurs; and
c) detecting or measuring an amount of any immunospecific binding by said antibody.

92. A diagnostic kit, comprising in one or more containers, a pair of primers, each having at least 15-25 nucleotides, in which at least one of said primers is hybridizable to SEQ. ID. NO: 43 or it complement and wherein said primers are capable of priming DNA synthesis in a nucleic acid amplification reaction.

93. A method for treating a disease state associated with a chromosome 14 abnormality in a mammal, preferably a human, suffering from said disease state associated with said chromosome 14 abnormality, comprising administering a therapeutically effective amount of a Tng2 antisense molecule or an anti-Tng2 antibody to said mammal.

94. The method of claim 93, wherein said disease state comprises a T-cell leukemia or lymphoma and said chromosome 14 abnormality comprises a t(14:14)(q11:q32) translocation or an inv (14)(q11:q32) inversion.
